(19) United States
(12) Patent Application Publication
(10) Pub. No.: US 2002/0055130 A1
(43) Pub. Date: May 9, 2002

(54) Method and product for regulating apoptosis

(75) Inventor: Gary L. Johnson, Boulder, CO (US)

Correspondence Address: LAHIVE & COCKFIELD, 28 STATE STREET, BOSTON, MA, 02109, US

(73) Assignee: National Jewish Center for Immunology and Respiratory Medicine

(21) Appl. No.: 09/858,754

(22) Filed: May 16, 2001

Related U.S. Application Data
(63) Continuation of application No. 09/023,130, filed on Feb. 13, 1998, now abandoned.
(60) Provisional application No. 60/039,740, filed on Feb. 14, 1997.

(51) Int. Cl. G01N 33/567; C07H 21/04; C12N 9/64; C12P 21/02; C12N 5/06

(52) U.S. Cl. 435/7.21; 435/226; 435/69.1; 435/325; 536/23.2

(57) ABSTRACT

The present invention relates to isolated MEKK1 proteins, nucleic acid molecules having sequences that encode such proteins, and antibodies raised against such proteins. The present invention also includes methods to use such proteins to regulate apoptosis. The invention provides active fragments of MEKK1 proteins that are generated upon cleavage of MEKK1 with a caspase protease. These active fragments are capable of stimulating apoptosis. Moreover, the invention provides protease-resistant forms of MEKK1 proteins, that are resistant to cleavage by caspase proteases and that are capable of inhibiting apoptosis. Still further, the invention provides methods for generating an active fragment of MEKK1, methods of identifying modulators of the apoptotic activity of an active fragment of MEKK1 and methods of identifying modulators of caspase-mediated cleavage of MEKK1.

### RELATED APPLICATIONS

This application claims the benefit of prior-filed U.S. provisional application Ser. No. 60/039,740, entitled “Method and Product for Regulating Cell Responsiveness to External Signals, filed Feb. 14, 1997, the entire contents of which are hereby incorporated by reference.

### GOVERNMENT FUNDING

This invention was made in part with government support under USPHS Grants DK37871, DK48845, CA58157 and GM30324, each awarded by the National Institutes of Health. The government has certain rights in this invention.

### FIELD OF THE INVENTION

This invention relates to isolated nucleic acid molecules encoding MEKK proteins, substantially pure MEKK proteins, and products and methods for regulating apoptosis in cells.

### BACKGROUND OF THE INVENTION

Mitogen-activated protein kinase (MAPKs) (also called extracellular signal-regulated kinases or ERKs) are rapidly activated in response to ligand binding by both growth factor receptors that are tyrosine kinases (such as the epidermal growth factor (EGF) receptor) and receptors that are coupled to heterotrimeric guanine nucleotide binding proteins (G proteins) such as the thrombin receptor. In addition, receptors like the T cell (TCR) and B cell (BCR) receptors are non-covalently associated with src family tyrosine kinases which activate MAPK pathways. Speicfic cytokines like tumor necrosis factor (TNFα) can also regulate MAPK pathways. The MAPKs appear to integrate multiple intracellular signals transmitted by various second messengers. MAPKs phosphorylate and regulate the activity of enzymes and transcription factors including the EGF receptor, Rsk 90, phospholipase A₂, c-Myc, c-Jun and Elk-1/TCF. Although the rapid activation of MAPKs by receptors that are tyrosine kinases is dependent on Ras, G protein-mediated activation of MAPK appears to occur through pathways dependent and independent of Ras.

Complementation analysis of the pheromone-induced signaling pathway in yeast has defined a protein kinase system that controls the activity of Spkl and Fus3-Kss1, the Schizosaccharomyces pombe and Saccharomyces cerevisiae homologs of MAPK (see for example, B. R. Cairns et al., Genes and Dev. 6, 1305 (1992); B. J. Stevenson et al., Genes and Dev. 6, 1293 (1992); S. A. Nadin-Davis et al., EMBO J. 7, 985 (1988); Y. Wang et al., Mol. Cell. Biol. 11, 3554 (1991). In S. cerevisiae, the protein kinase Ste7 is the upstream regulator of Fus3-Kss1 activity; the protein kinase Ste11 regulates Ste7. The S. pombe gene products Byr1 and Byr2 are homologous to Ste7 and Ste11, respectively. The MEK (MAPK Kinase or ERK Kinase) or MKK (MAP Kinase kinase) enzymes are similar in sequence to Ste7 and Byr1. The MEKs phosphorylate MAPKs on both tyrosine and threonine residues which results in activation of MAPK. The mammalian serine-threonine protein kinase Raf phosphorylates and activates MEK, which leads to activation of MAPK. Raf is activated in response to growth factor receptor tyrosine kinase activity and therefore Raf may activate MAPK in response to stimulation of membrane-associated tyrosine kinases. Raf is unrelated in sequence to Ste11 and Byr2. Thus, Raf may represent a divergence in mammalian cells from the pheromone-responsive protein kinase system defined in yeast. Cell and receptor specific differences in the regulation of MAPKs suggest that other Raf independent regulators of mammalian MEKs exist.

Certain biological functions, such as growth and differentiation, are tightly regulated by signal transduction pathways within cells. Signal transduction pathways maintain the balanced steady state functioning of a cell. Disease states can arise when signal transduction in a cell breaks down, thereby removing the tight control that typically exists over cellular functions. For example, tumors develop when regulation of cell growth is disrupted enabling a clone of cells to expand indefinitely. Because signal transduction networks regulate a multitude of cellular functions depending upon the cell type, a wide variety of diseases can result from abnormalities in such networks. Devastating diseases such as cancer, autoimmune diseases, allergic reactions, inflammation, neurological disorders and hormone-related diseases can result from abnormal signal transduction.

### SUMMARY OF THE INVENTION

The present invention relates to isolated MEKK1 proteins, nucleic acid molecules having sequences that encode such proteins, and antibodies raised against such proteins. The present invention also includes methods to use such proteins to regulate apoptosis. The invention provides active fragments of MEKK1 proteins that are generated upon cleavage of MEKK1 with a caspase protease. These active fragments are capable of stimulating apoptosis. Moreover, the invention provides protease-resistant forms of MEKK1 proteins, that are resistant to cleavage by caspase proteases and that are capable of inhibiting apoptosis. Still further, the invention provides methods for generating an active fragment of MEKK1, methods of identifying modulators of the apoptotic activity of an active fragment of MEKK1 and methods of identifying modulators of caspase-mediated cleavage of MEKK1.

It has been discovered that MEK kinase 1 (MEKK1), a 196 kDa protein kinase, functions to integrate proteases and signal transduction pathways involved in the regulation of apoptosis. Cleavage of mouse MEKK1 at Asp⁸⁷⁴ generates a 91 kDa kinase fragment and a 113 kDa NH₂-terminal fragment. The kinase fragment of MEKK1 induces apoptosis. Cleavage of MEKK1 and apoptosis are inhibited by p35 and CrmA, viral inhibitors of the ICE/FLICE proteases that commit cells to apoptosis. Mutation of the MEKK1 sequence ⁸⁷¹DTVD⁸⁷⁴, a cleavage site for CCP32-like proteases, to alanines inhibited proteolysis of MEKK1 and apoptosis induced by overexpression of MEKK1. Inhibition of MEKK1 proteolysis inhibited apoptosis but did not block MEKK1 stimulation of c-Jun kinase activity, indicating that c-Jun kinase activation was not sufficient for apoptosis. During the apoptotic response to UV irradiation, cisplatin, etoposide and mitomycin C, MEKK1 undergoes a phosphorylation-dependent activation followed by its proteolysis. These results show that MEKK1 activation and cleavage occurs in response to genotoxic agents and the activated kinase fragment functions to commit cells to apoptosis.

Accordingly, this invention defines MEKK1 as a protease substrate that when activated and cleaved stimulates an apoptotic response. The proteolytic cleavage of MEKK1 defines the mechanism to generate a protein kinase whose activity is sufficient to induce apoptosis. In the context of cancer therapy, the finding that the activation and cleavage of MEKK1 occurs in response to genotoxic agents is particularly important. It has been found that expression of MEKK1 is capable of killing by apoptosis cells that have both p53 alleles mutated. Hence, the activation and cleavage of MEKK1 is an apoptotic pathway that does not require a functional p53 and stimulation of these events could enhance the killing of many different tumors. Manipulating the activation of MEKK1 and its cleavage by proteases, with the use of drugs for example, could increase the killing of tumor cells to genotoxic agents. Consistent with this hypothesis is the finding that low level expression of MEKK1 potentiated the apoptotic response to low doses of UV irradiation and cisplatin.

Signal pathways involving MEKK1 in vertebrates, and the corresponding pathway in yeast, are illustrated in FIG. 1. The dual MEKK and Raf pathways are illustrated in FIG. 2. A mechanistic model of MEKK1 in apoptosis is illustrated in FIG. 8. The amino acid sequence of mouse full-length MEKK1 is shown in FIG. 9. A shorter MEKK1 protein is shown in SEQ ID NO: 2 (encoded by the nucleotide sequence of SEQ ID NO: 1). An alignment of the mouse, rat and human (partial) MEKK1 amino acid sequences is shown in FIG. 10. It should be noted that the caspase cleavage site DTVD (SEQ ID NO: 7), which is found at amino acids 871-874 of the mouse MEKK1 of FIG. 9, is conserved among all three of the mammalian MEKK1 proteins shown in FIG. 10. The nucleotide sequence of the full-length mouse MEKK1 cDNA is shown in FIGS. 11A-K.

One aspect of the present invention pertains to active fragments of MEKK1 proteins (i.e., fragments of MEKK1 proteins that retain apoptotic activity). Such active fragments are generated naturally be cleavage of MEKK1 by a caspase protease after a cleavage site found at amino acids 871-874 of FIG. 9. Alternatively, the active fragments of the invention can be prepared by recombinant DNA technology, using standard methodologies. In one embodiment, the invention provides an isolated active fragment of an MEKK1 protein consisting of an amino acid sequence having at least 75% homology to an amino acid sequence consisting of about amino acids 875-1493 of FIG. 9, wherein said active fragment mediates apoptosis. Preferably, the active fragment consists of an amino acid sequence having at least 85% homology to an amino acid sequence consisting of about amino acids 875-1493 of FIG. 9. More preferably, the active fragment consists of an amino acid sequence having at least 95% homology to an amino acid sequence consisting of about amino acids 875-1493 of FIG. 9. In one embodiment, the active fragment is a mouse MEKK1 active fragment. In another embodiment, the active fragment is a human MEKK1 active fragment. In another embodiment, the active fragment is a rat MEKK1 active fragment. The active fragment can consist of, for example, about amino acids 875-1493 of FIG. 9. Preferably, the active fragment consists of amino acids 875-1493 of FIG. 9.

Another aspect of the invention pertains to protease-resistant forms of MEKK1 proteins. Such protease-resistant forms can be generated by mutation of the caspase cleavage site in an MEKK1 protein corresponding to amino acids 871-874 of FIG. 9 such that the site cannot be cleaved by the caspase. Preferably, at least the Asp residue at 871 and/or 874 is mutated. Preferably, one or more of the amino acids corresponding to 871-874 of FIG. 9 can be mutated to, for example, alanine residues. Alternatively, Asp871 and/or Asp874 can be mutated to glutamine. Accordingly, the invention provides an isolated protease-resistant MEKK1 protein comprising an amino acid sequence having at least 75% homology to the amino acid sequence of FIG. 9, wherein at least one amino acid equivalent to amino acids 871-874 of FIG. 9 is substituted such that the MEKK1 protein is resistant to proteolysis by a caspase after amino acid 874. In one embodiment, at least one amino acid equivalent to amino acids 871-874 of FIG. 9 is substituted with an alanine residue. In another embodiment, each amino acid equivalent to amino acids 871-874 of FIG. 9 is substituted with an alanine residue. Preferably, the protease-resistant MEKK1 protein has at least 85% homology to the amino acid sequence of FIG. 9. More preferably, the protease-resistant MEKK1 protein has at least 95% homology to the amino acid sequence of FIG. 9. In one embodiment, the protease-resistant MEKK1 protein is a mouse MEKK1 protein. In another embodiment, the protease-resistant MEKK1 protein is a human MEKK1 protein. In yet another embodiment, the protease-resistant MEKK1 protein is a rat MEKK1 protein.

The invention further provides isolated nucleic acid molecules that encode the MEKK1 active fragments of the invention. In one embodiment, the invention provides an isolated nucleic acid molecule consisting of a nucleotide sequence having at least 75% homology to a nucleotide sequence consisting of about nucleotides 645-2501 of SEQ ID NO: 1, wherein said nucleic acid molecule encodes an active fragment of MEKK1 that mediates apoptosis. Preferably, the nucleic acid molecule consists of a nucleotide sequence having at least 85% homology to a nucleotide sequence consisting of about nucleotides 645-2501 of SEQ ID NO: 1. More preferably, the nucleic acid molecule consists of a nucleotide sequence having at least 95% homology to a nucleotide sequence consisting of about nucleotides 645-2501 of SEQ ID NO: 1. In one embodiment, the nucleic acid molecule encodes an active fragment of mouse MEKK1. In another embodiment, the nucleic acid molecule encodes an active fragment of human MEKK1. In yet another embodiment, the nucleic acid molecule encodes an active fragment of rat MEKK1. In a preferred embodiment, the nucleic acid molecule consists of about nucleotides 645-2501 of SEQ ID NO: 1, or a nucleotide sequence that, due to the degeneracy of the genetic code, encodes the same amino acid sequence as about nucleotides 645-2501 of SEQ ID NO: 1. In another preferred embodiment, the nucleic acid molecule consists of nucleotides 645-2501 of SEQ ID NO: 1, or a nucleotide sequence that, due to the degeneracy of the genetic code, encodes the same amino acid sequence as nucleotides 645-2501 of SEQ ID NO:1.

The invention also provides isolated nucleic acid molecules encoding the protease-resistant forms of MEKK1 of the invention. For example, the invention provides an isolated nucleic acid molecule encoding a protease-resistant MEKK1 protein, wherein the protease resistant MEKK1 protein comprises an amino acid sequence having at least 75% homology to the amino acid sequence of FIG. 9 and at least one codon of the nucleic acid molecule encoding an amino acid equivalent to at least one of amino acids 871-874 of FIG. 9 is mutated such the encoded MEKK1 protein is resistant to proteolysis by a caspase after an amino acid equivalent to amino acid 874 of FIG. 9. Preferably, the MEKK1 protein comprises an amino acid sequence having at least 85% homology to the amino acid sequence of FIG. 9. More preferably, the MEKK1 protein comprises an amino acid sequence having at least 95% homology to the amino acid sequence of FIG. 9. In one embodiment, the nucleic acid encodes a protease-resistant mouse MEKK1 protein. In another embodiment, the nucleic acid encodes a protease-resistant human MEKK1 protein. In yet another embodiment, the nucleic acid molecule encodes a protease-resistant rat MEKK1 protein.

Expression vector that comprise the isolated nucleic acid molecules of the invention, and host cells that comprise the expression vectors, are also encompassed by the invention.

Yet another aspect of the invention pertains to methods for modulating apoptosis. In one embodiment, the invention provides a method of stimulating apoptosis in a cell comprising introducing into the cell an expression vector encoding an MEKK1 active fragment of the invention such that MEKK1 active fragment is produced in the cell and apoptosis is stimulated. In another embodiment, the invention provides a method of inhibiting apoptosis in a cell comprising introducing into the cell an expression vector encoding a protease-resistant MEKK1 protein of the invention such that protease-resistant MEKK1 protein is produced in the cell and apoptosis is inhibited.

The invention also provides methods for generating MEKK1 active fragments in vitro. For example, an MEKK1 active fragment can be generated in vitro by:

contacting an MEKK1 protein in vitro with a caspase protease under proteolysis conditions; and

allowing the caspase protease to cleave the MEKK1 protein such that an MEKK1 active fragment is generated.

Preferably, the caspase protease is a caspase-3 protease. Alternatively, the caspase protease is a caspase-7 protease. Standard proteolysis conditions known in the art under which caspase proteases are known to be active can be used in the method of the invention.

Still another aspect of the invention pertains to methods for identifying modulators of apoptosis. In one embodiment, the invention provides a method of identifying a compound that modulates the apoptotic activity of an MEKK1 active fragment. The method comprises:

providing an indicator cell that comprises an MEKK1 active fragment of the invention;

contacting the indicator cell with a test compound; and

determining the effect of the test compound on the apoptotic activity of the MEKK1 active fragment in the indicator cell to thereby identify a compound that modulates the apoptotic activity of the MEKK1 active fragment.

The indicator cell may naturally express an MEKK1 active fragment or may be transfected with an expression vector that encodes the MEKK1 active fragment such that the active fragment is expressed in the cell. The effect of the test compound can be evaluated, for example, by measuring an apoptotic response in the cells, such as DNA fragmentation.

In another embodiment, the invention provides a method of identifying a compound that modulates the proteolytic cleavage of an MEKK1 protein by a caspase protease, comprising:

providing a reaction mixture that comprises an MEKK1 protein and a caspase protease;

contacting the reaction mixture with a test compound; and

determining the effect of the test compound on proteolytic cleavage of the MEKK1 protein by the caspase protease to thereby identify a compound that modulates the proteolytic cleavage of an MEKK1 protein by a caspase protease.

Preferably, the caspase protease is a caspase-3 protease. Alternatively, the caspase protease is a caspase-7 protease. Standard proteolysis conditions known in the art under which caspase proteases are known to be active can be used in the method of the invention. The effect of the test compound on the proteolytic cleavage of MEKK1 can be evaluated by, for example, monitoring the generation of the 91 kD active fragment of MEKK1 (e.g., by detection of the 91 kD fragment using an anti-MEKK1 antibody, using standard techniques).

### BRIEF DESCRIPTION OF THE FIGURES

FIG. 1 is a schematic representation of the signal pathways of vertebrates and yeast.

FIG. 2 is a schematic representation of the dual MEKK and Raf pathways divergent from Ras protein pathway.

FIG. 3 is a bar graph showing quantitation of the percentage of MEKK1 -transfected cells in the presence or in the absence of the caspase inhibitors CrmA or p35, that showed DNA fragmentation as an indication of apoptosis. The data represents the number of cells transfected with MEKK1 that were counted on at least four coverslips from at least two different experiments.

FIG. 4 is a photograph of a Western blot analysis of lysates using the 12CA5 and 95-012 antibodies, demonstrating that CrmA and p35 inhibit the generation of a MEKK1-derived, kinase active, cleavage product. Fragments A, B, C, and D correspond to MEKK1 cleavage products and the band marked with an asterisks may correspond to a dimer of fragment D.

FIG. 5 is a schematic representation of the HA-tagged mouse MEKK1 protein showing the regions (the numbers correspond to the position of the amino acids) used to generate the indicated antibodies. Also shown is the sequence (one letter code) between amino acids 853 and 888 where the tetrapeptides DEVE (SEQ ID NO: 6) and DTVD (SEQ ID NO: 7) (in bold) have been replaced with alanine residues in mutants DEVE→A and DTVD→A, respectively.

FIG. 6 is a schematic representation of the p35-inhibitable and p35-insensitive cleavage in the mouse MEKK1 protein. The letters A to D indicate the names of the cleavage products. The molecular weights were calculated from the migration of the markers in at least 2 different experiments.

FIG. 7 is a bar graph showing quantitation of the percentage of MEKK1 DEVE→A or DTVD→A mutant-transfected cells that showed DNA fragmentation as an indication of apoptosis. The numbers in the columns indicate the number of cells transfected with the MEKK1 mutants that were counted on at least four coverslips from at least two different experiments.

FIG. 8 is a schematic diagram of a mechanistic model of MEKK1-induced apoptosis.

FIG. 9 shows the amino acid sequence of full-length mouse MEKK1 protein (SEQ ID NO: 3).

FIG. 10A-10B shows the alignment of mouse MEKK1 (SEQ ID NO: 3), rat MEKK1 (SEQ ID NO: 4) and human MEKK1 (SEQ ID NO: 5) proteins. The conserved DTVD (SEQ ID NO: 7) caspase cleavage site is boxed.

FIG. 11A-K shows the nucleotide sequence of full-length mouse MEKK1 protein (SEQ ID NO: 13).

### DETAILED DESCRIPTION OF THE INVENTION

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Through a series of inducible and reversible protein-protein interactions and phosphorylation-mediated enzymatic activities, regulatory proteins are recruited to relay signals throughout the cell. Such interactions are involved in all stages of the intracellular signal transduction process—at the plasma membrane, where the signal is initiated; in the cytoplasm, where the signals are disseminated to different cellular locations; and in the nucleus, where other proteins involved in transcriptional control form complexes to regulate transcription of particular genes. The structural nature of protein interactions and control of enzymatic activities in signal transduction is emerging through the identification of the individual proteins that participate in each signal transduction pathway, the elucidation of the temporal order in which these proteins interact, and the definition of the sites of contact between the proteins. The understanding gained in intracellular signaling pathways of cells will be advantageous in developing the next generation of pharmaceuticals. In particular, the pleiotropic richness of intracellular signaling pathways in cells represents a means for developing more selective pharmacological activity in a therapeutic agent than may be possible in the present generation of drugs.

The present invention concerns the discovery of a family of novel mitogen ERK kinase kinase proteins (referred to herein as “MEK kinases”, “MEKKs” or “MEKK proteins”) which function in intracellular signal transduction pathways in a variety of cells, and accordingly have a role in determining cell/tissue fate and maintenance. The family of MEKK genes or gene products provided by the present invention apparently consists of at least six different members (MEKK4.2 is a splicing varient of MEKK4.1 and MEKK2.2 is a sequencing varient of MEKK2) with ample evidence indicating that yet other members of the family exist.

A salient feature of the MEKK gene products deriving from this discovery not only implicates these proteins in intracellular signaling, but also strongly suggests that the diversity of the MEKK family is important to providing a diversity of responses to different environmental cues. That is, the ability of a cell to respond to a particular growth factor, morphogen, or even stress cue, and the type of response the cell undegoes is dependent at least in part upon which MEKK gene products are expressed in the cell and/or engaged by signals propagated upstream of the kinase.

Still another important feature of the present invention is the discovery of the involvment of MEKK proteins in certain apoptotic pathways.

Accordingly, certain aspects of the present invention relate to nucleic acids encoding vertebrate MEKK proteins, the MEKK proteins themselves, antibodies immunoreactive with MEKK proteins, and preparations of such compositions. Moreover, the present invention provides diagnostic and therapeutic assays and reagents for detecting and treating disorders involving, for example, aberrant expression or activation of the MEKK gene products. In addition, drug discovery assays are provided for identifying agents which can modulate the biological function of MEKK proteins, such as by altering the binding of the protein to either downstream or upstream elements in a signal transduction pathway, or which inhibit the kinase activity of the MEKK protein. Such agents can be useful therapeutically to alter the growth and/or differentiation of a cell. Other aspects of the invention are described below or will be apparent to those skilled in the art in light of the present disclosure.

Intial cloning of a member of the mammalian MEKK family was accomplished using primers based on sequences for the yeast protein kinases Byr2 (from S pombe) and Ste11 (from S. cerevisiae). Using the sequence obtained for the mammalian MEKK cDNA, other MEKK transcripts have been detected and several subsequently cloned to reveal a family of mammalian MEKK proteins.

The primary sequence of the MEKK proteins suggests two functional domains, an amino-terminal moiety rich in serine and threonine that apparently serves a regulatory role, and a carboxy-terminal protein kinase catalytic domain. The catalytic domain of, for example, MEKK1 shows approximately 35 percent identity with the amino acid sequences of the catalytic domains of Byr2 and Ste11. The amino-terminal moieties of each of the mammalian MEKKs show little similarity with Ste11 or Byr2.

Furthermore, the MEKK family is apparently encoded by several genes, at least some of which are able to produce different transcripts by differential splicing. For example, the divergence in sequence amongst the catalytic domains of each of MEKK1 to MEKK4 indicated that seperate genomic genes encode each paralog. However, MEKK2 and MEKK4 genes can give rise to at least two different transcripts, presumably be differential splicing. Expression data suggests that MEKKs 1-4 are ubiquitously expressed.

By use of overexpression and/or constitutively activated MEKKs, a variety of cellular substrates for each MEKK protein have been identified. In general, the proteins of the MAP kinase kinases (MEK) family are each targets for one or more of the MEKKs. Moreover, the data set out below demonstrate that MEKK-dependent signal propagation can result in the phosphorylation/activation of members of the MAP kinase family, such as p42MAPK, p44MAPK, p38MAPK, and the Jun NH₂-terminal kinases (JNKs).

Certain of the MEKK proteins have been shown to be activated, e.g., as kinases, in response to growth factors and cytokines (such as TNFα and chemoattractants like FMLP and IL-8) and other environmental cues, including stress, as well as expression of activated Ras or other members of the Ras Superfamily, including Rac and Cdc 42. It is demonstrated below that the kinase domain of at least MEKK1 binds to activated Ras in a GTP-dependent manner, implicating that interaction as a a potential therapeutic target. Moreover, a Ras effector domain peptide blocks the binding of the MEKK catalytic domain with the GTP-bound form of Ras.

Yet another set of experimental data provided in the appended examples indicates that activation of certain MEKK pathways can lead to apoptosis. The integration of signal transduction pathways regulated by growth factor and cytokine receptors commits a cell either to proliferation or apoptosis (Sumimoto, S. L. et al. (1994) J. Immunol. 153:2488-2496). Specific cytokines and stresses to cells, such as DNA damage, appear to preferentially acitvate the JNK/SAPK pathway which leads to apoptosis. Several checkpoints exist in the pathways leading to apoptosis that involve proteins such as Bcl2 and p58, which can both inhibit apoptosis. The MEKK proteins are therefore, important to the dynamic balance between growth factor-activated ERK and stress-activated JNK/p38 pathways and accordingly important in determining whether a cell survives or undergoes apoptosis. To date candidate molecules involved in signaling apoptosis include ceramide, Ras, Rho, c-myc, c-Jun, and the proteins associated with the TNFα receptor and Fas.

One aspect of the present invention relates to isolated MEKK proteins. As used herein protein, peptide, and polypeptide are meant to be synonomous. According to the present invention, an isolated protein is a protein that has been removed from its natural milieu. It will be understood that “isolated”, with respect to MEKK polypeptides, is meant to include formulations of the polypeptides which are isolated from, or otherwise substantially free of other cellular proteins (“contaminating proteins”), especially other cellular signal transduction factors, normally associated with the MEKK polypeptide. Thus, isolated MEKK protein preparations include preparations having less than 20% (by dry weight) contaminating protein, and preferably having less than 5% contaminating protein (but water, buffers, and other small molecules, especially molecules having a molecular weight of less than 5000, can be present). Functional forms of the subject MEKK polypeptides can be prepared, for the first time, as purified preparations by using a cloned gene as described herein. Alternatively, the subject MEKK polypeptides can be isolated by affinity purification using, for example, a catalytically inactive MEK. “Isolated” does not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

An isolated MEKK protein can, for example, be obtained from its natural source, be produced using recombinant DNA technology, or be synthesized chemically. As used herein, an isolated MEKK protein can be a full-length MEKK protein or any homologue of such a protein, such as a MEKK protein in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycosylphosphatidyl inositol), wherein the modified protein is capable of phosphorylating MAP kinase kinases, such as mitogen ERK kinases (MEKs (MKK1 and MKK2)) and/or Jun kinase kinases (JNKKs (MKK3 and MKK4)).

As used herein, the term “gene” or “recombinant gene” refers to a nucleic acid comprising an open reading frame encoding one of the MEKK polypeptides of the present invention, including both exon and (optionally) intron sequences. A “recombinant gene” refers to nucleic acid encoding a vertebrate MEKK polypeptide and comprising vertebrate MEKK-encoding exon sequences, though it may optionally include intron sequences which are either derived from a chromosomal vertebrate MEKK gene or from an unrelated chromosomal gene. Exemplary recombinant genes encoding the subject vertebrate MEKK polypeptide are represented in the appended Sequence Listing. The term “intron” refers to a DNA sequence present in a given vertebrate MEKK gene which is not translated into protein and is generally found between exons.

A homologue of a MEKK protein is a protein having an amino acid sequence that is sufficiently similar to a natural MEKK protein amino acid sequence that a nucleic acid sequence encoding the homologue is capable of hybridizing under stringent conditions to (i.e., with) a nucleic acid sequence encoding the natural MEKK protein amino acid sequence. As used herein, stringent hybridization conditions refer to standard hybridization conditions under which nucleic acid molecules, including oligonucleotides, are used to identify similar nucleic acid molecules. Such standard conditions are disclosed, for example, in Sambrook et al., Molecular Cloning: A Laboratory Manual, Cold Spring Harbor Labs Press, 1989. A homologue of a MEKK protein also includes a protein having an amino acid sequence that is sufficiently cross-reactive such that the homologue has the ability to elicit an immune response against at least one epitope of a naturally-occurring MEKK protein.

The minimal size of a protein homologue of the present invention is a size sufficient to be encoded by a nucleic acid molecule capable of forming a stable hybrid with the complementary sequence of a nucleic acid molecule encoding the corresponding natural protein. As such, the size of the nucleic acid molecule encoding such a protein homologue is dependent on nucleic acid composition, percent homology between the nucleic acid molecule and complementary sequence, as well as upon hybridization conditions per se (e.g., temperature, salt concentration, and formamide concentration). The minimal size of such nucleic acid molecules is typically at least about 12 to about 15 nucleotides in length if the nucleic acid molecules are GC-rich and at least about 15 to about 17 bases in length if they are AT-rich. As such, the minimal size of a nucleic acid molecule used to encode a MEKK protein homologue of the present invention is from about 12 to about 18 nucleotides in length. There is no limit, other than a practical limit, on the maximal size of such a nucleic acid molecule in that the nucleic acid molecule can include a portion of a gene, an entire gene, or multiple genes, or portions thereof. Similarly, the minimal size of a MEKK protein homologue of the present invention is from about 4 to about 6 amino acids in length, with preferred sizes depending on whether a full-length, multivalent protein (i.e., fusion protein having more than one domain each of which has a function), or a functional portion of such a protein is desired.

MEKK (K protein homologues can be the result of allelic variation of a natural gene encoding a MEKK protein. A natural gene refers to the form of the gene found most often in nature. MEKK protein homologues can be produced using techniques known in the art including, but not limited to, direct modifications to a gene encoding a protein using, for example, classic or recombinant DNA techniques to effect random or targeted mutagenesis. As will be understood, mutagenesis includes point mutations, as well as deletions and truncations of the MEKK polypeptide sequence. The ability of a MEKK protein homologue to phosphorylate MEK and/or JNKK protein can be tested using techniques known to those skilled in the art. Such techniques include phosphorylation assays described in detail in the Examples section.

With respect to homologues, it will also be possible to modify the structure of the subject MEKK polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the MEKK polypeptide described in more detail herein. Such modified peptide can be produced, for instance, by amino acid substitution, deletion, or addition.

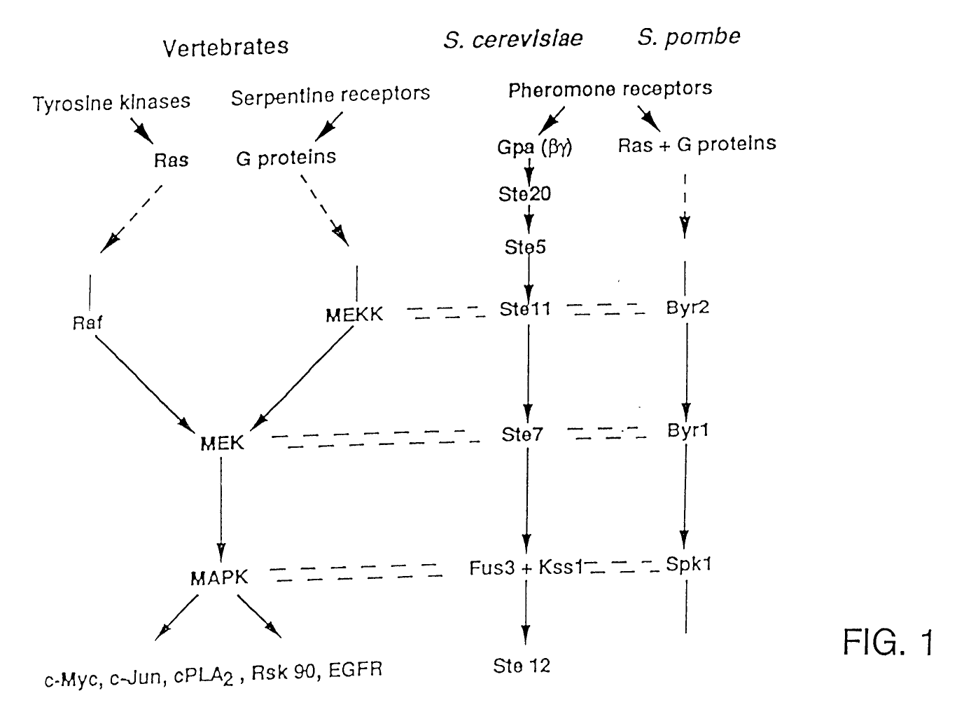

In one embodiment, a MEKK protein of the present invention is capable of regulating a MEKK-dependent pathway. According to the present invention, a MEKK-dependent pathway refers generally to a pathway in which a MEKK protein regulates a pathway substantially independent of Raf, though the pathway including the MEKK protein may converge with common members of a pathway involving Raf protein, such as a MEK protein (see FIG. 1).

In certain preferred embodiments, the MEKK protein will be involved in a pathway controlling the phosphorylation of a mitogen-activiated protein (MAP) kinase. The mammalian MAP kinase family includes, for example, the extracellular signal-regulated protein kinases (ERK1 and ERK2), p42 or p44 MAPKs. In another preferred embodiment the MEKK protein will be involved in the pathway controlling c-Jun NH2-terminal kinases (JNKs, or SAPKs), and the so-called “p38 subgroup” kinases (p38 and Hog-1 kinases). For example, it is contemplated that the MEKK proteins of the present invention interact with, and directly phosphorylate members of the MAP kinase kinase family (MEKs or MKKs), as MEK1, MEK2, MKK1, MKK2, or the stress-activated kinases (SEKs), and the Jun kinase kinases (JNKK1, JNKK2, MKK3, MKK4), or the like.

An exemplary MEKK-dependent pathway includes a pathway involving a MEKK protein and a MKK protein. One of skill in the art can determine whether or not the regulation of a pathway by a MEKK protein is substantially independent of a Raf protein by comparing the ability of a MEKK protein and a Raf protein to regulate the phosphorylation of a downstream member of such pathway. For instance, a MEKK protein can regulate a pathway substantially independently of a Raf protein if the MEKK protein induces phosphorylation of a member of the pathway downstream of MEKK (e.g., proteins including JEK, Jun kinase, Jun and/or ATF-2) by an amount significantly greater than that seen when Raf protein is utilized. Raf-1 and B-Raf kinases selectively regulate MEK1 and MEK2 and do not recognize the JNKK proteins, thus Raf proteins appear to be highly selective for the regulation of p42/p44 MAPK pathways. MEKK proteins, in contrast, are capable of regulating both JNK and p42/p44 MAPK pathways.

For example, MEKK induction of phosphorylation of a JNK protein is preferably at least about 10-fold, more preferably at least about 20-fold and even more preferably at least about 30-fold than the phosphorylation of the JNK protein induced when using a Raf protein. If MEKK induction of phosphorylation is similar to Raf protein induction of phosphorylation, then one of skill in the art can conclude that regulation of a pathway by a MEKK protein includes members of a signal transduction pathway that could also include Raf protein. For example, MEKK induction of phosphorylation of MAPK is of a similar magnitude as induction of phosphorylation with Raf protein.

A “Raf-dependent pathway” refers to a signal transduction pathway in which a Raf protein regulates a signal transduction pathway substantially independently of a MEKK protein, and a pathway in which Raf protein regulation converges with common members of a pathway involving MEKK protein. The independence of regulation of a pathway by a Raf protein from regulation of a pathway by a MEKK protein can be determined using methods similar to those used to determine MEKK independence.

In another embodiment, a MEKK protein is capable of regulating the activity of signal transduction proteins including, but not limited to, mitogen activated ERK kinases (MEKs), mitogen activated protein kinases (MAPKs), transcription control factor (TCF), Ets-like-1 transcription factor (Elk-1), Jun ERK kinases (JNKKs), Jun kinases (JNK; which is equivalent to SAPK), stress activated MAPK proteins, Jun, activating transcription factor-2 (ATF-2) and/or Myc protein. As used herein, the “activity” of a protein can be directly correlated with the phosphorylation state of the protein and/or the ability of the protein to perform a particular function (e.g., phosphorylate another protein or regulate transcription). Preferred MEK proteins regulated by a MEKK protein of the present invention include MEK-1 and/or MEK-2 (MKK1 or MKK2). Preferred MAPK proteins regulated by a MEKK protein of the present invention include p38/Hog-1 MAPK, p42 MAPK and/or p44 MAPK. Preferred stress activated MAPK proteins regulated by a MEKK protein of the present invention include Jun kinase (JNK), stress activated MAPK-α and/or stress activated MAPK-β.

A MEKK protein of the present invention is capable of increasing the activity of an MEK protein over basal levels of MEK (i.e., levels found in nature when not stimulated). For example, a MEKK protein is preferably capable of increasing the phosphorylation of an MEK protein (such as MEK1 or MEK2, also known as MKK1 and MKK2 respectively) by at least about 2-fold, more preferably at least about 3-fold, and even more preferably at least about 4-fold over basal levels when measured under conditions. In another embodiment, a preferred MEKK protien is capable of increasing the phosphorylation of a JNKK protein (such as JNKK1 or JNKK2, also known as MKK3 and MKK4 respecitvely).

A preferred MEKK protein of the present invention is also capable of increasing the activity of an MAPK protein over basal levels of MAPK (i.e., levels found in nature when not stimulated). For example, a MEKK protein of the present invention is preferably capable of increasing MAPK activity at least about 2-fold, more preferably at least about 3-fold, and even more preferably at least about 4-fold over basal activity.

Moreover, a MEKK protein of the present invention is capable of increasing the activity of a JNK protein. JNK regulates the activity of the transcription factor JUN which is involved in controlling the growth and differentiation of different cell types, such as T cells, neural cells or fibroblasts. JNK also regulates Elk-1, an Ets family member. JNK shows structural and regulatory homologies with MAPK. For example, a MEKK protein of the present invention is preferably capable of inducing the phosphorylation of JNK protein at least about 30 times more than Raf, more preferably at least about 40 times more than Raf, and even more preferably at least about 50 times more than Raf.

In addition, a MEKK protein of the present invention is capable of specific binding to a Ras superfamily protein. In particular, a MEKK protein is capable of binding to a Ras protein that is associated with GTP. According to the present invention, a MEKK protein binds to Ras via the COOH terminal region of the MEKK protein, e.g., a ras-binding domain.

In a preferred embodiment, a MEKK protein of the present invention is capable of phosphorylating a MEK or MKK, Jun kinase kinase (JNKK) and/or stress activated ERK kinase (SEK), in particular MEK1, MEK2, MKK1, MKK2, MKK3, MKK4, JNKK1, JNKK2, SEK1 and/or SEK2 proteins. As described herein, MEK1 and MEK2 are equivalent to MKK1 and MKK2, respectively. In addition, JNKK1 and JNKK2 are equivalent to MKK3 and MKK4, which are equivalent to SEK1 and SEK2.

A preferred MEKK protein of the present invention is additionally capable of inducing the phosphorylation of a Myc protein, particularly a transcriptional transactivation domain of Myc, in such a manner that the phosphorylated Myc protein is capable of regulating gene transcription. For example, a MEKK protein of the present invention is preferably capable of inducing luciferase gene transcription by a phosphorylated Myc at least about 25-fold, more preferably at least about 35-fold, and even more preferably at least about 45-fold, over Raf induction.

Another aspect of the present invention relates to the ability of a MEKK activity to be stimulated by growth factors including, but not limited to, epidermal growth factor (EGF), neuronal growth factor (NGF), tumor necrosis factor (TNF), C5A, interleukin-8 (IL-8), interleukin-5 (IL-5), monocyte chemotactic protein 1 (MIP1α), monocyte chemoattractant protein 1 (MCP-1), platelet activating factor (PAF), N-Formyl-methionyl-leucyl-phenylalanine (FMLP), leukotriene B₄ (LTB₄R), gastrin releasing peptide (GRP), IgE, major histocompatibility protein (MHC), peptide, superantigen, antigen, vasopressin, thrombin, bradykinin and acetylcholine. In addition, the activity of a MEKK protein of the present invention is capable of being stimulated by compounds including phorbol esters such as TPA. A preferred MEKK protein is also capable of being stimulated by EGF, NGF and/or TNF (especially TNFα).

Preferably, the activity of certain of the MEKK proteins of the present invention is capable of being stimulated at least 2-fold over basal levels (i.e., levels found in nature when not stimulated), more preferably at least about 4-fold over basal levels and even more preferably at least about 6-fold over basal levels, when a cell producing the MEKK protein is contacted with EGF.

Similarly, the activity of certain of the MEKK proteins of the present invention are capable of being stimulated at least 1-fold over basal levels, more preferably at least about 2-fold over basal levels and even more preferably at least about 3-fold over basal levels by NGF stimulation, when a cell producing the MEKK protein is contacted with NGF under the conditions described in the appended examples. MEKK proteins which are stimulated by NGF may subsequently cause the activation of one or more ERKs.

On the other hand, as demonstrated below, certain of the MEKK proteins of the present invention are capable of being stimulated by removal of NGF stimulation. MEKK proteins which are stimulated by NGF removal may subsequently cause the activation of one or more p38 kinases and/or JNKs.

In yet another embodiment, a MEKK protein of the present invention is capable of being stimulated at least 0.5-fold over basal levels, more preferably at least about 1-fold over basal levels and even more preferably at least about 2-fold over basal levels by TPA stimulation when a cell producing the MEKK protein is contacted with TPA.

TNF is capable of regulating cell death and other functions in different cell types. Another aspect of the present invention relates to the discovery that MEKK stimulation by TNF can be independent of Raf. Similarly, the present invention demonstrates that the kinase activity of certain of the subject MEKK proteins can be stimulated by ultraviolet light (UV) damage of cells. It has been observed that both TNF and UV stimulate MEKK activity without substantially activating Raf. In addition, both UV and TNF activation of MEKK is apparently Ras dependent. In certain embodiments FGF is capable of preventing TNF induced apoptosis.

Another aspect of the present invention is the recognition that a MEKK protein of the present invention is capable of regulating the apoptosis of a cell As used herein, apoptosis refers to the form of cell death that comprises: progressive contraction of cell volume with the preservation of the integrity of cytoplasmic organelles; condensation of chromatin, as viewed by light or electron microscopy; and DNA cleavage, as electrophoresis or labeling of DNA fragments using terminal deoxytransferase (TDT). Cell death occurs when the membrane integrity of the cell is lost and cell lysis occurs. Apoptosis differs from necrosis in which cells swell and eventually rupture.

A preferred MEKK protein of the present invention is capable of inducing the apoptosis of cells, such that the cells have characteristics substantially similar to cytoplasmic shrinkage and/or nuclear condensation as described in the apended Examples. The appended examples also illustrate that TNF and MEKK can synergize to induce apoptosis in cells.

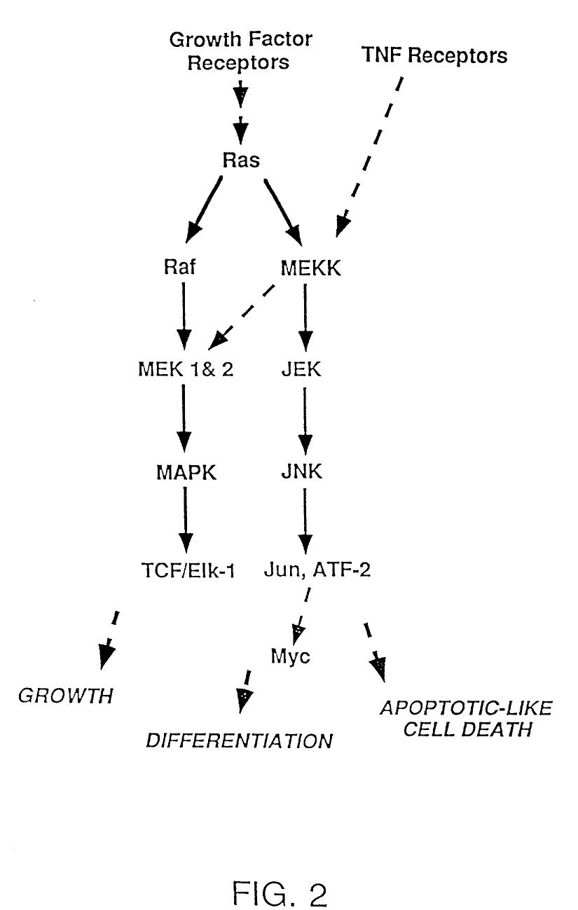

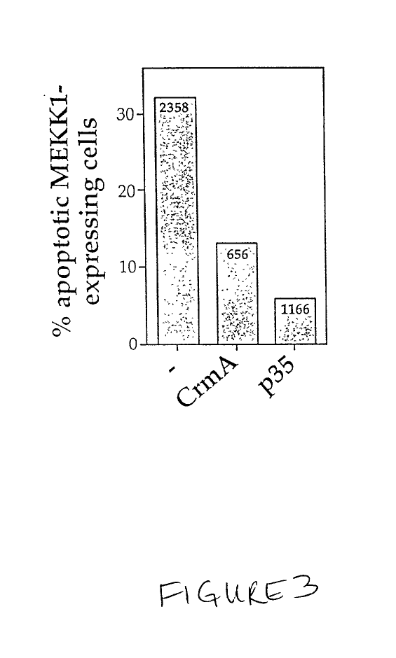

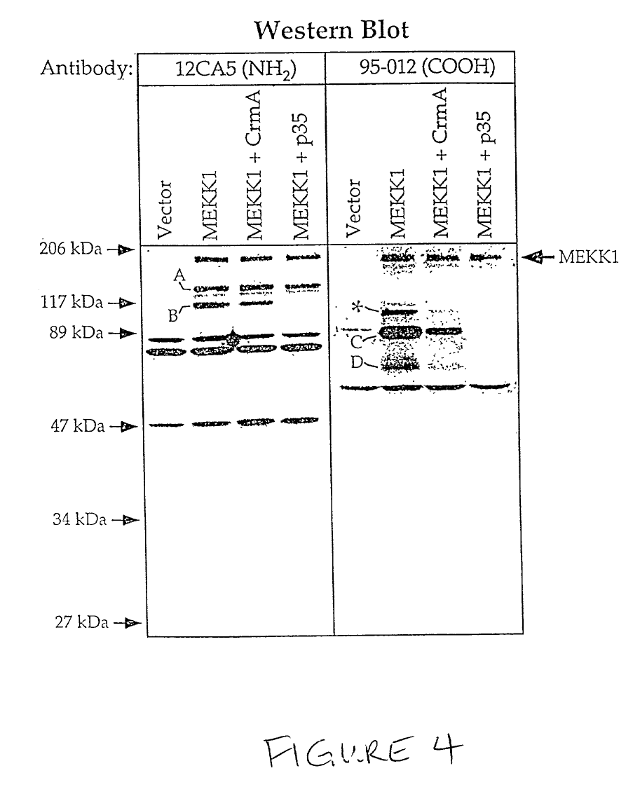

A schematic representation of an exemplary cell growth regulatory signal transduction pathway that is MEKK dependent is shown in FIG. 2. Preferred MEKK proteins of the present invention are capable of regulating the activity of a JNKK protein, JNK protein, Jun protein and/or ATF-2 protein, and Myc protein, such regulation being substantially, if not entirely, independent of Raf protein. Such Raf-independent regulation can regulate the growth characteristics of a cell, including the apoptosis of a cell. In addition, a MEKK protein of the present invention is capable of regulating the activity of MEK protein, which is also capable of being regulated by Raf protein. As such, a MEKK protein of the present invention is capable of regulating the activity of MAPK protein and members of the Ets family of transcription factors, such as TCF protein, also referred to as Elk-1 protein.

Referring to FIG. 2, a MEKK protein of the present invention is capable of being activated by a variety of growth factors and environmental cues capable of activating Ras superfamily protein. In addition, a MEKK protein is capable of activating JNK protein which is also activated by Ras protein, but which is not activated by Raf protein. As such, a MEKK protein of the present invention comprises a protein kinase at a divergence point in a signal transduction pathway initiated by different cell surface receptors. A MEKK protein is also capable of being regulated by TNF protein independent of Raf, thereby indicating an association of MEKK protein to a novel signal transduction pathway which is independent of Ras protein and Raf protein.

Thus, a MEKK protein is capable of performing numerous unique functions independent of or by-passing Raf protein in one or more signal transduction pathways. A MEKK protein is capable of regulating the activity of MEK and/or JNKK activity. As such, a MEKK protein is capable of regulating the activity of members of a signal transduction pathway that does not substantially include Raf activity. Such members include, but are not limited to, JNK, Jun, ATF and Myc protein. In addition, a MEKK protein is capable of regulating the members of a signal transduction pathway that does involve Raf, such members including, but are not limited to, MEK, MAPK and TCF. A MEKK protein of the present invention is thus capable of regulating the apoptosis of a cell independent of significant involvement by Raf protein.

In addition to the numerous functional characteristics of a MEKK protein, a MEKK protein of the present invention comprises numerous unique structural characteristics. For example, in one embodiment, a MEKK protein of the present invention includes at least one of two different structural domains having particular functional characteristics. Such structural domains include an NH₂-terminal regulatory domain that serves to regulate a second structural domain comprising a COOH-terminal protein kinase catalytic domain that is capable of phosphorylating an MKK protein.

According to the present invention, a MEKK protein of the present invention includes a full-length MEKK protein, as well as at least a portion of a MEKK protein capable of performing at least one of the functions defined above. The phrase “at least a portion of a MEKK protein” refers to a portion of a MEKK protein encoded by a nucleic acid molecule that is capable of hybridizing, under stringent conditions, with a nucleic acid encoding a full-length MEKK protein of the present invention. Preferred portions of MEKK proteins are useful for regulating apoptosis in a cell. Additional preferred portions have activities useful for regulating MEKK kinase activity. Suitable sizes for portions of a MEKK protein of the present invention are as disclosed for MEKK protein homologues of the present invention.

In another embodiment, a MEKK protein of the present invention includes at least a portion of a MEKK protein having molecular weights ranging from about 70 kD to about 250 kD as determined by Tris-glycine SDS-PAGE, preferably using an 8% polyacrylamide SDS gel (SDS-PAGE) and resolved using methods standard in the art. A preferred MEKK protein has a molecular weight ranging from about 65 kD to about 190 kD and even more preferably from about 69 kD to about 98 kD. In particularly preferred embodiments MEKK2 and MEKK3 proteins of the present invention have a molecular weight of about 65-75 kD. Preferred MEKK4 proteins have molecular weights about 180-190 kD. Most preferred molecular weights for the subject MEKKs are: >175 kD (MEKK1), 69.5 kD (MEKK2 or MEKK2.2), 71 kD (MEKK3), 185 kD (MEKK4). It is noted that experimental conditions used when running gels to determine the molecular size of putative MEKK proteins will cause variations in results. Moreover, it has become apparent to the Applicant that, relative to predicted molecular weights, shorter apparently related polypeptides can be observed. Whether these are the result of proteolytic processing, alternative splicing or start codon usage or the like is unclear, but other preferred MEKK1 polypeptides (e.g. MEKK1.2) have apparent molecular weights of about 95-100 kD; and other preferred MEKK4 polypeptides (e.g., MEKK4.2) have apparent molecular weights of about 90-100 kD, more preferably 95-98 kD.

In another embodiment, an NH₂-terminal regulatory domain of the present invention includes an NH₂-terminal comprising about 400 amino acids having at least about 10% serine and/or threonine residues, more preferably about 400 amino acids having at least about 15% serine and/or threonine residues, and even more preferably about 400 amino acids having at least about 20% serine and/or threonine residues.

In another embodiment an NH₂-terninal regulatory domain of the present invention includes an NH₂-terminal comprising about 600 amino acids having at least about 10% serine and/or threonine residues, more preferably about 600 amino acids having at least about 15% serine and/or threonine residues, and even more preferably about 600 amino acids having at least about 20% serine and/or threonine residues.

Another preferred an NH₂-terminal regulatory domain of the present invention includes an NH₂-terminal comprising about 1300 amino acids having at least about 10% serine and/or threonine residues, more preferably about 1300 amino acids having at least about 15% serine and/or threonine residues, and even more preferably about 1300 amino acids having at least about 20% serine and/or threonine residues.

In other embodiments certain MEKK proteins comprise pleckstrin homology domains. The ‘pleckstrin homology’ (PH) domain is an approximately 100-residue protein module that is thought to be involved in interactions with GTP-binding proteins (Musacchio et al (1993) TIBS 28:343-348). Pleckstrin homology domains are very divergent and do not occupy a specific positions in molecules; alignments of PH domains show six conserved blocks which all contain several conserved hydrophobic residues which are thought to form a folded structure comprising seven to eight P-strands, most likely in one or two β-sheets, and just a single helix (Musacchio et al. supra). PH domains have been identified in kinases and also in Vav, Dbl, Bcr, yeast cdc24, Ras-GAP, DM GAP, Ras-GRF, and Sos, all of which are regulators of small GTP-binding proteins. Interestingly, three of the four proteins that have been identified as being capable of binding to SH3 domains (dynamin, 3BP2, and Sos) also contain PH domains (Musacchio et al. supra). The PH domain of β adrenergic receptor kinase may be involved in binding to G protein βγ complexes (Koch et al. (1993) J. Biol. Chem. 268:8256-8260).

The sequences comprising the catalytic domain of a MEKK protein are involved in phosphotransferase activity, and therefore display a relatively conserved amino acid sequence. The NH₂-terminal regulatory domain of a MEKK protein, however, can be substantially divergent. The lack of significant homology between MEKK protein NH₂-terminal regulatory domains is related to the regulation of each of such domains by different upstream regulatory proteins. For example, a MEKK protein can be regulated by the protein Ras, while others car be regulated independent of Ras. In addition, some MEKK proteins can be regulated by the growth factor TNFα, while others cannot. As such, the NH₂-terminal regulatory domain of a MEKK protein provides selectivity for upstream signal transduction regulation, while the catalytic domain provides for MEKK substrate selectivity function.

In another embodiment, the subject MEKK proteins are provided as fusion proteins. It is widely appreciated that fusion proteins can also facilitate the expression of proteins, and accordingly, can be used in the expression of the MEKK polypeptides of the present invention. For example, MEKK polypeptides can be generated as glutathione-S-transferase (GST-fusion) proteins. Such GST-fusion proteins can enable easy purification of the MEKK polypeptide, as for example by the use of glutathione-derivatized matrices (see, for example, Current Protocols in Molecular Biology, eds. Ausubel et al. (N.Y.: John Wiley & Sons, 1991)).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni2+ metal resin. The purification leader sequence can then be subsequently removed by treatment with enterokinase to provide the purified protein (e.g., see Hochuli et al. (1987) J. Chromatography 411:177; and Janknecht et al. PNAS 88:8972).

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

According to the present invention, a MEKK protein of the present invention can include MEKK proteins that have undergone post-translational modification. Such modification can include, for example, phosphorylation or among other post-translational modifications including conformational changes or post-translational deletions.

This invention further contemplates a method for generating sets of combinatorial mutants of the subject MEKK proteins as well as truncation mutants, and is especially useful for identifying potential variant sequences (e.g. homologs) that are functional in modulating signal transduction. The purpose of screening such combinatorial libraries is to generate, for example, novel MEKK homologs which can act as either agonists or antagonist of the wild-type MEKK proteins, or alternatively, which possess novel activities all together. To illustrate, MEKK homologs can be engineered by the present method to provide selective, constitutive activation of a pathway, so as mimic induction by a factor when the MEKK homolog is expressed in a cell capable of responding to the factor. Thus, combinatorially-derived homologs can be generated to have an increased potency relative to a naturally occurring form of the protein.

Likewise, MEKK homologs can be generated by the present combinatorial approach to selectively inhibit (antagonize) induction by a growth or other factor. For instance, mutagenesis can provide MEKK homologs which are able to bind other signal pathway proteins (e.g., MEKs) yet prevent propagation of the signal, e.g. the homologs can be dominant negative mutants. Moreover, manipulation of certain domains of MEKK by the present method can provide domains more suitable for use in fusion proteins.

In one aspect of this method, the amino acid sequences for a population of MEKK homologs or other related proteins are aligned, preferably to promote the highest homology possible. Such a population of variants can include, for example, MEKK homologs from one or more species. Amino acids which appear at each position of the aligned sequences are selected to create a degenerate set of combinatorial sequences. In a preferred embodiment, the variegated library of MEKK variants is generated by combinatorial mutagenesis at the nucleic acid level, and is encoded by a variegated gene library. For instance, a mixture of synthetic oligonucleotides can be enzymatically ligated into gene sequences such that the degenerate set of potential MEKK sequences are expressible as individual polypeptides, or alternatively, as a set of larger fusion proteins (e.g. for phage display) containing the set of MEKK sequences therein.

There are many ways by which such libraries of potential MEKK homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential MEKK sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for a MEKK clone in order to generate a variegated population of MEKK fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of a MEKK coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with SI nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of MEKK homologs. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate MEKK sequences created by combinatorial mutagenesis techniques.

In an illustrative embodiment of a screening assay, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. Coli filamentous phages M13, fd, and f1 are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461). The resulting phage libraries with the fusion tail proteins may be panned, e.g. using a fluorescently labeled MEK protein, e.g. FITC-MEK, to score for MEKK homologs which retain the ability to bind to the MEK protein. Individual phage which encode a MEKK homolog which retains MEK binding can be isolated, the MEKK homolog gene recovered from the isolate, and further tested to discern between active and antagonistic mutants.

In another embodiment, the REF52 cells can be exploited to analyze the variegated MEKK library. For instance, the library of expression vectors can be transfected into a population of REF52 cells which also inducibly overexpress a MEKK protein (e.g., and which overexpression causes apoptosis). Expression of WT-MEKK is then induced. and the effect of the MEKK mutant on induction of apoptosis can be detected. Plasmid DNA can then be recovered from the cells which score for inhibition, or alternatively, potentiation of apoptosis, and the individual clones further characterized.

The invention also provides for reduction of the MEKK proteins to generate mimetics, e.g. peptide or non-peptide agents, which are able to disrupt binding of a MEKK polypeptide of the present invention with either upstream or downstream components of its signaling cascade. Thus, such mutagenic techniques as described above are also useful to map the determinants of the MEKK proteins which participate in protein-protein interactions involved in, for example, binding of the subject MEKK polypeptide to proteins which may function upstream (including both activators and repressors of its activity) or to proteins which may function downstream of the MEKK polypeptide, whether they are positively or negatively regulated by it. To illustrate, the critical residues of a subject MEKK polypeptide which are involved in molecular recognition of an upstream or downstream MEKK component can be determined and used to generate MEKK-derived peptidomimetics which competitively inhibit binding of the authentic protein with that moiety. By employing, for example, scanning mutagenesis to map the amino acid residues of each of the subject MEKK proteins which are involved in binding other cellular proteins, peptidomimetic compounds can be generated which mimic those residues of the MEKK protein which facilitate the interaction. Such mimetics may then be used to interfere with the normal function of a MEKK protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

Another aspect of the present invention is an isolated nucleic acid molecule capable of hybridizing, under stringent conditions, with a MEKK protein gene encoding a MEKK protein of the present invention. In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. To this end, the term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject MEKK polypeptides preferably includes no more than 10 kilobases (kb) of nucleic acid sequence which naturally immediately flanks the MEKK gene in genomic DNA, more preferably no more than 5 kb of such naturally occurring flanking sequences, and most preferably less than 1.5 kb of such naturally occurring flanking sequence. The term isolated as used herein will also be understood to include nucleic acid that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

An isolated nucleic acid molecule can include DNA, RNA, or derivatives of either DNA or RNA. Accordingly, as used herein, the term “nucleic acid” includes polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” includes nucleic acid comprising an open reading frame encoding one of the MEKK polypeptides of the present invention, including both exon and (optionally) intron sequences. A “recombinant gene” refers to nucleic acid encoding a MEKK polypeptide and comprising MEKK-encoding exon sequences, though it may optionally include intron sequences which are either derived from a chromosomal MEKK gene or from an unrelated chromosomal gene. Exemplary recombinant genes encoding the subject MEKK polypeptides are represented in the appended Sequence Listing.

An isolated nucleic acid moleculc of the present invention can be obtained from its natural source either as an entire (i.e., complete) gene or a portion thereof capable of forming a stable hybrid with that gene. As used herein, the phrase “at least a portion of” an entity refers to an amount of the entity that is at least sufficient to have the functional aspects of that entity. For example, at least a portion of a nucleic acid sequence, as used herein, is an amount of a nucleic acid sequence capable of forming a stable hybrid with a particular desired gene (e.g., MEKK genes) under stringent hybridization conditions. An isolated nucleic acid molecule of the present invention can also be produced using recombinant DNA technology (e.g., polymerase chain reaction (PCR) amplification, cloning) or chemical synthesis. Isolated MEKK protein nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode a MEKK protein of the present invention or to form stable hybrids under stringent conditions with natural nucleic acid molecule isolates of MEKK.

Preferred modifications to a MEKK protein nucleic acid molecule of the present invention include truncating a full-length MEKK protein nucleic acid molecule by, for example: deleting at least a portion of a MEKK protein nucleic acid molecule encoding a regulatory domain to produce a constitutively active MEKK protein; deleting at least a portion of a MEKK protein nucleic acid molecule encoding a catalytic domain to produce an inactive MEKK protein; and modifying the MEKK protein to achieve desired inactivation and/or stimulation of the protein, for example, substituting a codon encoding a lysine residue in the catalytic domain (i.e., phosphotransferase domain) with a methionine residue to inactivate the catalytic domain.

A preferred truncated MEKK nucleic acid molecule encodes a form of a MEKK protein containing a catalytic domain but that lacks a regulatory domain. Preferred catalytic domain truncated MEKK nucleic acid molecules encode amino acid residues from about 409 to about 672 of MEKK1.1; amino acids 1331 to about 1594 of MEKK1.2; from about 361 to about 620 of MEKK2.1 or 2.2; from about 366 to about 626 of MEKK3; from about 631 to about 890 of MEKK4.1; or from about 1338 to about 1597 for MEKK4.2.

Another preferred truncated MEKK nucleic acid molecule encodes a form of a MEKK protein comprising an NH₂-terminal regulatory domain a catalytic domain but lacking a catalytic domain. Preferred regulatory domain truncated MEKK nucleic acid molecules encode amino acid residues from about 1 to about 408 of MEKK1.1; amino acids 1 to about 1328 of MEKK1.2; from about 1 to about 360 of MEKK2.1 or 2.2.; from about 1 to about 365 of MEKK3; from about 1 to about 630 of MEKK4.1; or from about 1 to about 1337 for MEKK4.2.

An isolated nucleic acid molecule of the present invention can include a nucleic acid sequence that encodes at least one MEKK protein of the present invention, examples of such proteins being disclosed herein. Although the phrase “nucleic acid molecule” primarily refers to the physical nucleic acid molecule and the phrase “nucleic acid sequence” primarily refers to the sequence of nucleotides that comprise the nucleic acid molecule, the two phrases can be used interchangeably. As heretofore disclosed, MEKK proteins of the present invention include, but are not limited to, proteins having full-length MEKK protein coding regions, portions thereof, and other MEKK protein homologues.

As used herein, a MEKK protein gene includes all nucleic acid sequences related to a natural MEKK protein gene such as regulatory regions that control production of a MEKK protein encoded by that gene (including, but not limited to, transcription, translation or post-translation control regions) as well as the coding region itself. A nucleic acid molecule of the present invention can be an isolated natural MEKK protein nucleic acid molecule or a homologue thereof. A nucleic acid molecule of the present invention can include one or more regulatory regions, full-length or partial coding regions, or combinations thereof. The minimal size of a MEKK protein nucleic acid molecule of the present invention is the minimal size capable of forming a stable hybrid under stringent hybridization conditions with a corresponding natural gene.

A MEKK protein nucleic acid molecule homologue can be produced using a number of methods known to those skilled in the art (see, e.g., Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., the ability of a homologue to phosphorylate MEK protein or JNKK protein) and/or by hybridization with isolated MEKK protein nucleic acids under stringent conditions.

A preferred nucleic acid molecule of the present invention is capable of hybridizing under stringent conditions to a nucleic acid that encodes at least a portion of a MEKK protein, or a homologue thereof. Also preferred is a MEKK nucleic acid molecule that includes a nucleic acid sequence having at least about 50% homology, preferably 75% homology, preferably 85% homology, or even more preferably 95% homology with an MEKK nucleic acid molecule of the invention. In other embodiments nucleic acids have 50%, preferably at least about 75%, and more preferably at least about 85%, and most preferably at least about 95% homology with the corresponding region(s) of the nucleic acid sequence encoding the catalytic domain of a MEKK protein, or a homologue thereof. Also preferred is a MEKK protein nucleic acid molecule that includes a nucleic acid sequence having at least about 50%, preferably at least about 75%, more preferably at least about 85%, and even more preferably at least about 95% homology with the corresponding region(s) of the nucleic acid sequence encoding the NH₂-terminal regulatory domain of a MEKK protein, or a homologue thereof. Such nucleic acid molecules can be a full-length gene and/or a nucleic acid molecule encoding a full-length protein, a hybrid protein, a fusion protein, a multivalent protein or a truncation fragment.

Knowing a nucleic acid molecule of a MEKK protein of the present invention allows one skilled in the art to make copies of that nucleic acid molecule as well as to obtain additional portions of MEKK protein-encoding genes (e.g., nucleic acid molecules that include the translation start site and/or transcription and/or translation control regions), and/or MEKK protein nucleic acid molecule homologues. Knowing a portion of an amino acid sequence of a MEKK protein of the present invention allows one skilled in the art to clone nucleic acid sequences encoding such a MEKK protein.

The present invention also includes nucleic acid molecules that are oligonucleotides capable of hybridizing, under stringent conditions, with complementary regions of other, preferably longer, nucleic acid molecules of the present invention that encode at least a portion of a MEKK protein, or a homologue thereof. A preferred oligonucleotide is capable of hybridizing, under stringent conditions, with a nucleic acid molecule of SEQ ID NO: 1.

Oligonucleotides of the present invention can be RNA, DNA, or derivatives of either. The minimal size of such oligonucleotides is the size required to form a stable hybrid between a given oligonucleotide and the complementary sequence on another nucleic acid molecule of the present invention. Minimal size characteristics of preferred oligonucleotides are at least about 10 nuclotides, preferably at least about 20 nucleotides, more preferably at least about 50 nucleotides and most preferably at least about 60 nucleotides. Larger fragments are also contemplated. The size of the oligonucleotide must also be sufficient for the use of the oligonucleotide in accordance with the present invention. Oligonucleotides of the present invention can be used in a variety of applications including, but not limited to, as probes to identify additional nucleic acid molecules, as primers to amplify or extend nucleic acid molecules or in therapeutic applications to inhibit, for example, expression of MEKK proteins by cells. Such therapeutic applications include the use of such oligonucleotides in, for example, antisense-, triplex formation-, ribozyme- and/or RNA drug-based technologies. The present invention, therefore, includes use of such oligonucleotides and methods to interfere with the production of MEKK proteins. In addition cligonucleotides encoding portions of MEKK proteins which bind to MEKK binding proteins can be used a therapeutics. In other embodiments, the peptides encoded by these nucleic acids are used.

To further illustrate, another aspect of the invention relates to the use of the isolated nucleic acid in “antisense” therapy. As used herein, “antisense” therapy refers to administration or in situ generation of oligonucleotide probes or their derivatives which specifically hybridize (e.g. bind) under cellular conditions, with the cellular mRNA and/or genomic DNA encoding one or more of the subject MEKK proteins so as to inhibit expression of that protein, e.g. by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a vertebrate MEKK protein. Alternatively, the antisense construct is an oligonucleotide probe which is generated ex vivo and which, when introduced into the cell causes inhibition of expression by hybridizing with the mRNA and/or genomic sequences of a vertebrate MEKK gene. Such oligonucleotide probes are preferably modified oligonucleotides which are resistant to endogenous nucleases, e.g. exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general. For such therapy, the oligomers of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

In addition to use in therapy, the oligomers of the invention may be used as diagnostic reagents to detect the presence or absence of the target DNA or RNA sequences to which they specifically bind. Such diagnostic tests are described in further detail below.

Likewise, the antisense constructs of the present invention, by antagonizing the normal biological activity of one of the MEKK proteins, can be used in the manipulation of tissue, e.g. tissue differentiation, both in vivo and for ex vivo tissue cultures.

Furthermore, the anti-sense techniques (e.g. microinjection of antisense molecules, or transfection with plasmids whose transcripts are anti-sense with regard to a MEKK mRNA or gene sequence) can be used to investigate role of MEKK in disease states, as well as the normal cellular function of MEKK in healthy tissue. Such techniques can be utilized in cell culture, but can also be used in the creation of transgenic animals. The present invention also includes a recombinant vector which includes at least one MEKK protein nucleic acid molecule of the present invention inserted into any vector capable of delivering the nucleic acid molecule into a host cell. Such a vector contains heterologous nucleic acid sequences, for example nucleic acid sequences that are not naturally found adjacent to MEKK protein nucleic acid molecules of the present invention. The vector can be either RNA or DNA, and either prokaryotic or eukaryotic, and is typically a virus or a plasmid. Recombinant vectors can be used in the cloning, sequencing, and/or otherwise manipulating of MEKK protein nucleic acid molecules of the present invention. One type of recombinant vector, herein referred to as a recombinant molecule and described in more detail below, can be used in the expression of nucleic acid molecules of the present invention. Preferred recombinant vectors are capable of replicating in the transformed cell.

Preferred nucleic acid molecules to insert into a recombinant vector includes a nucleic acid molecule that encodes at least a portion of a MEKK protein, or a homologue thereof. In particularly preferred embodiments portions of a MEKK nucleic acid which encodes a MEKK catalytic domain is used. In another particularly preferred embodiment, at least a portion of a nucleic acid which encodes the portion of a MEKK protein which binds to a MEKK substrate or a MEKK regulatory protein is used.

Suitable host cells for transforming a cell can include any cell capable of producing MEKK proteins of the present invention after being transformed with at least one nucleic acid molecule of the present invention. Host cells can be either untransformed cells or cells that are already transformed with at least one nucleic acid molecule. Suitable host cells of the present invention can include bacterial, fungal (including yeast), insect, animal and plant cells. Preferred host cells include bacterial, yeast, insect and mammalian cells, with mammalian cells being particularly preferred.

A recombinant cell is preferably produced by transforming a host cell with one or more recombinant molecules, each comprising one or more nucleic acid molecules of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, insect, animal, and/or plant cells. As such, nucleic acid molecules of the present invention can be operatively linked to expression vectors containing regulatory sequences such as promoters, operators, repressors, enhancers, termination sequences, origins of replication, and other regulatory sequences that are compatible with the recombinant cell and that control the expression of nucleic acid molecules of the present invention. As used herein, a transcription control sequence includes a sequence which is capable of controlling the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which functioh in bacterial, yeast, and mammalian cells, such as, but not limited to, tac, lac, trp, trc, oxy-pro, omp/lpp, rrnB, bacteriophage lambda (λ) (such as λp_{L} and λp_{R} and fusions that include such promoters), bacteriophage T7, T7lac, bacteriophage T3, bacteriophage SP6, bacteriophage SP01, metallothionein, alpha mating factor, baculovirus, vaccinia virus, herpesvirus, poxvirus, adenovirus, simian virus 40, retrovirus actin, retroviral long terminal repeat, Rous sarcoma virus, heat shock, phosphate and nitrate transcription control sequences, as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with a DNA sequence encoding a MEKK protein.

Preferred nucleic acid molecules for insertion into an expression vector include nucleic acid molecules that encode at least a portion of a MEKK protein, or a homologue thereof.

Expression vectors of the present invention may also contain fusion sequences which lead to the expression of inserted nucleic acid molecules of the present invention as fusion proteins. Inclusion of a fusion sequence as part of a MEKK nucleic acid molecule of the present invention can enhance the stability during production, storage and/or use of the protein encoded by the nucleic acid molecule. Furthermore, a fusion segment can function as a tool to simplify purification of a MEKK protein, such as to enable purification of the resultant fusion protein using affinity chromatography. A suitable fusion segment can be a domain of any size that has the desired function (e.g., increased stability and/or purification tool). It is within the scope of the present invention to use one or more fusion segments. Fusion segments can be joined to amino and/or carboxyl termini of a MEKK protein. Linkages between fusion segments and MEKK proteins can be constructed to be susceptible to cleavage to enable straight-forward recovery of the MEKK proteins. Fusion proteins are preferably produced by culturing a recombinant cell transformed with a fusion nucleic acid sequence that encodes a protein including the fusion segment attached to either the carboxyl and/or amino terminal end of a MEKK protein.

Moreover, the gene constructs of the present invention can also be used as a part of a gene therapy protocol to deliver nucleic acids encoding either an agonistic or antagonistic form of one of the subject MEKK proteins. Thus, another aspect of the invention features expression vectors for in vivo or in vitro transfection and expression of a MEKK polypeptide in particular cell types so as to reconstitute the function of, constituitively activate, or alternatively, abrogate the function of a signal pathway dependent on a MEKK activity. Such therapies may useful where the naturally-occurring form of the protein is misexpressed or inappropriately activated; or to deliver a form of the protein which alters differentiation of tissue; or which inhibits neoplastic transformation.

Expression constructs of the subject MEKK polypeptide, a id mutants thereof, may be administered in any biologically effective carrier, e.g. any formulation or composition capable of effectively delivering the recombinant gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that the particular gene construct provided for in vivo transduction of MEKK expression are also useful for in vitro transduction of cells, such as for use in the ex vivo tissue culture systems described below.

A preferred approach for in vivo introduction of nucleic acid into a cell is by use of a viral vector containing nucleic acid, e.g. a cDNA, encoding the particular MEKK polypeptide desired. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including neuronal cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad Sci. USA 87:6141-6145; Huber et al. (1991) Proc. Natl. Acad. Sci. USA 88:8039-8043; Ferry et al. (1991) Proc. Natl. Acad Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Neda et al. (1991) J. Biol Chem 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the MEKK gene of the retroviral vector.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Biotechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including airway epithelium (Rosenfeld et al. (1992) cited supra), endothelial cells (Lemarchand et al. (1992) Proc. Natl. Acad Sci. USA 89:6482-6486), hepatocytes (Herz and Gerard (1993) Proc. Natl. Acad Sci. USA 90:2812-2816) and muscle cells (Quantin et al. (1992) Proc. Natl. Acad. Sci. USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the inserted MEKK gene can be under control of, for example, the E1A promoter, the major late promoter (MLP) and associated leader sequences, the E3 promoter, or exogenously added promoter sequences.

Yet another viral vector system useful for delivery of one of the subject MEKK genes is the adeno-associated virus (AMINO ACIDSV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AMINO ACIDSV can be packaged and can integrate. Space for exogenous DNA is limited to about 4.5 kb. An AMINO ACIDSV vector such as that described in Tratschin et al. (1985) Mol. Cell. Biol. 5:3251-3260 can be used to introduce DNA into cells. A variety of nucleic acids have been introduced into different cell types using AMINO ACIDSV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a subject MEKK polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject MEKK polypeptide gene by the targeted cell. Exemplary gene delivery systems of this type include liposomal derived systems, polylysine conjugates, and artificial viral envelopes.

In clinical settings, the gene delivery systems for the therapeutic MEKK gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057). A MEKK gene, such as any one of the clones represented in the appended Sequence Listing, can be delivered in a gene therapy construct by electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

Still another aspect of the present invention pertains to recombinant cells, e.g., cells which are transformed with at least one of any nucleic acid molecule of the present invention. A preferred recombinant cell is a cell transformed with at least one nucleic acid molecule that encodes at least a portion of a MEKK protein, or a homologue thereof.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgamo sequences), modification of nucleic acid molecules of the present invention to correspond to the codon usage of the host cell, deletion of sequences that destabilize transcripts, and use of control signals that temporally separate recombinant cell growth from recombinant protein production during fermentation. The activity of an expressed recombinant protein of the present invention may be improved by fragmenting, modifying, or derivatizing the resultant protein.

As used herein, amplifying the copy number of a nucleic acid sequence in a cell can be accomplished either by increasing the copy number of the nucleic acid sequence in the cell's genome or by introducing additional copies of the nucleic acid sequence into the cell by transformation. Copy number amplification is conducted in a manner such that greater amounts of enzyme are produced, leading to enhanced conversion of substrate to product. For example, recombinant molecules containing nucleic acids of the present invention can be transformed into cells to enhance enzyme synthesis. Transformation can be accomplished using any process by which nucleic acid sequences are inserted into a cell. Prior to transformation, the nucleic acid sequence on the recombinant molecule can be manipulated to encode an enzyme having a higher specific activity.

In accordance with the present invention, recombinant cells can be used to produce a MEKK protein of the present invention by culturing such cells under conditions effective to produce such a protein, and recovering the protein. Effective conditions to produce a protein include, but are not limited to, appropriate media, bioreactor, temperature, pH and oxygen conditions that permit protein production. An appropriate, or effective, medium refers to any medium in which a cell of the present invention, when cultured, is capable of producing a MEKK protein. Such a medium is typically an aqueous medium comprising assimilable carbohydrate, nitrogen and phosphate sources, as well as appropriate salts, minerals, metals and other nutrients, such as vitamins. The medium may comprise complex nutrients or may be a defined minimal medium.

A preferred cell to culture is a recombinant cell that is capable of expressing the MEKK protein, the recombinant cell being produced by transforming a host cell with one or more nucleic acid molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

With respect to methods for producing the subject MEKK polypeptide, a host cell transfected with a nucleic acid vector directing expression of a nucleotide sequence encoding the subject polypeptides can be cultured under appropriate conditions to allow expression of the peptide to occur. The cells may be harvested, lysed and the protein isolated. A cell culture includes host cells, media and other byproducts. Suitable media for cell culture are well known in the art. The recombinant MEKK polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant MEKK polypeptide is a fusion protein containing a domain which facilitates its purification, such as GST fusion protein or poly(His) fusion protein.

Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant MEKK proteins may either remain within the recombinant cell or be secreted into the fermentation medium. The phrase “recovering the protein” refers simply to collecting the whole fermentation medium containing the protein and need not imply additional steps of separation or purification. MEKK proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization.

Alternatively, a MEKK protein of the present invention can be produced by isolating the MEKK protein from cells or tissues recovered from an animal that normally express the MEKK protein. For example, a cell type, such as T cells, can be isolated from the thymus of an animal. MEKK protein can then be isolated from the isolated primary T cells using standard techniques described herein.

The availability of purified and recombinant MEKK polypeptides as described in the present invention facilitates the development of assays which can be used to screen for drugs, including MEKK homologs, which are either agonists or antagonists of the normal cellular function of the subject MEKK polypeptides, or of their role in the pathogenesis of cellular differentiation and/or proliferation, and disorders related thereto. In one embodiment, the assay evaluates the ability of a compound to modulate binding between a MEKK polypeptide and a molecule that interacts either upstream or downstream of the MEKK polypeptide in the a cellular signaling pathway. A variety of assay formats will suffice and, in light of the present inventions, will be comprehended by a skilled artisan.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements. Accordingly, in an exemplary screening assay of the present invention, the compound of interest is contacted with proteins which may function upstream (including both activators and repressors of its activity such as, Ras, Rac, Cdc 42 or Rho or other Ras superfamily members) or to proteins or nucleic acids which may function downstream of the MEKK polypeptide, whether they are positively or negatively regulated by it. For convenience, such polypeptides of a signal transduction pathway which interact directly with MEKK will be referred to below as MEKK-binding proteins (MEKK-bp). These proteins include the downstream targets of MEKKs, namely, members of the MAP kinase kinase family (MEKs or MKKs), as MEKI, MEK2, MKK1, MKK2, the stress-activated kinases (SEKs), also known as the Jun kinase kinases (JNKKs), MEKK3 and MEKK4 or the like. Other downstream targets of the MEKK family can include proteins from the mammalian MAP kinase family which includes, for example, the extracellular signal-regulated protein kinases (ERKs), c-Jun NH₂-terminal kinases (JNKs, or SAPKs), and the so-called “p38 subgroup” kinases (p38 kinases).

To the mixture of the compound and the MEKK-bp is then added a composition containing a MEKK polypeptide. Detection and quantification of complexes including MEKK and the MEKK-bp provide a means for determining a compound's efficacy at inhibiting (or potentiating) complex formation between MEKK and the MEKK-binding protein. The efficacy of the compound can be assessed by generating dose response curves from data obtained using various concentrations of the test compound. Moreover, a control assay can also be performed to provide a baseline for comparison. In the control assay, isolated and purified MEKK polypeptide is added to a composition containing the MEKK-binding protein, and the formation of a complex is quantitated in the absence of the test compound.

In an exemplary embodiment the Ras effector domain or MEKK4 or MEKK4.2 sequence IIGQVCDTPKSYDNVMHVGLR is used to inhibit the interaction of a MEKK protein with a MEKK binding protein.

Complex formation between the MEKK polypeptide and a MEKK-binding protein may be detected by a variety of techniques. Modulation of the formation of complexes can be quantitated using, for example, detectably labeled proteins such as radiolabeled, fluorescently labeled, or enzymatically labeled MEKK polypeptides, by immunoassay, or by chromatographic detection.

Typically, it will be desirable to immobilize either MEKK or its binding protein to facilitate separation of complexes from uncomplexed forms of one or both of the proteins, as well as to accommodate automation of the assay. Binding of the two proteins, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/MEKK (GST/MEKK) fusion proteins can be adsorbed onto glutathione sepharose beads (Sigma Chemical, St. Louis, Mo.) or glutathione derivatized microtitre plates, which are then combined with the MEKK-bp, e.g. an ³⁵S-labeled, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of MEKK-binding protein found in the bead fraction quantitated from the gel using standard electrophoretic techniques such as described in the appended examples.

Other techniques for immobilizing proteins on matrices are also available for use in the subject assay. For instance, either MEKK or its cognate binding protein can be immobilized utilizing conjugation of biotin and streptavidin. For instance, biotinylated MEKK molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with MEKK but which do not interfere with binding of upstream or downstream elements can be derivatized to the wells of the plate, and MEKK trapped in the wells by antibody conjugation. As above, preparations of a MEKK-binding protein and a test compound are incubated in the MEKK-presenting wells of the plate, and the amount of complex trapped in the well can be quantitated. Exemplary methods for detecting such complexes, in addition to those described above for the GST-immobilized complexes, include immunodetection of complexes using antibodies reactive with the MEKK binding protein, or which are reactive with the MEKK protein and compete with the binding protein; as well as enzyme-linked assays which rely on detecting an enzymatic activity associated with the binding protein, either intrinsic or extrinsic activity. In the instance of the latter, the enzyme can be chemically conjugated or provided as a fusion protein with the MEKK-bp. To illustrate, the MEKK-bp can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1 -chloro-2,4-dinitrobenzene (Habig et al (1974) J Biol Chem 249:7130).

For processes which rely on immunodetection for quantitating one of the proteins trapped in the complex, antibodies against the protein, such as anti-MEKK antibodies, can be used. Alternatively, the protein to be detected in the complex can be “epitope tagged” in the form of a fusion protein which includes, in addition to the MEKK sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, N.J.).

In addition to cell-free assays, such as described above, the readily available source of vertebrate MEKK proteins provided by the present invention also facilitates the generation of cell-based assays for identifying small molecule agonists/antagonists and the like. Cells which are sensitive to MEKK-mediated signal transduction events can be caused to overexpress a recombinant MEKK protein in the presence and absence of a test agent of interest, with the assay scoring for modulation in MEKK-dependent responses by the target cell mediated by the test agent. As with the cell-free assays, agents which produce a statistically signifcant change in MEKK-dependent signal transduction (either inhibition or potentiation) can be identified.

For example, as described in the appended examples, overexpression of MEKK1 and MEKK3 (and possibly MEKK2 and MEKK4) in certain cells can cause constitutive induction of apoptotic pathways and result in cell death. Accordingly, such recombinant cells can be used to identify inhibitors of MEKK protein signaling by the compound's ability to inhibit signal transduction events downstream of the MEKK proteins and thereby rescue the cell from apoptosis. To illustrate, the recombinant MEKK1 cells of Example 18 or 19 can be contacted with a panel of test compounds, and inhibitors scored by the ability to rescue the cells from an apoptotic fate (such as may be detected by use of dyes such as Hoechst 33258). Compounds which cause a statistically significant decrease in apoptosis of the MEKK1-overexpressing cells can be selected for further testing.

In another embodiment of a drug screening, a two hybrid assay can be generated with a MEKK and MEKK-binding protein. This assay permits the detection of protein-protein interactions in yeast such that drug dependent inhibition or potentiation of the interaction can be scored. As an illustrative example, GAL4 protein is a potent activator of transcription in yeast grown on galactose. The ability of GAL4 to activate transcription depends on the presence of an N-terminal sequence capable of binding to a specific DNA sequence (UASG) and a C-teminal domain containing a transcriptional activator. A sequence encoding a MEKK protein, “A”, may be fused to that encoding the DNA binding domain of the GAL4 protein. A second hybrid protein may be created by fusing sequence encoding the GAL4 transactivation domain to sequence encoding a MEKK-bp, “B”. If protein “A” and protein “B” interact, that interaction serves to bring together the two domains of GAL4 necessary to activate transcription of a UASG-containing gene. In addition to co-expressing plasmids encoding both hybrid proteins, yeast strains appropriate for the detection of protein-protein interactions would contain, for example, a GAL 1 -lacZ fusion gene to permit detection of transcription from a UASG sequence. Other examples of two-hybrid assays or interaction trap assays are known in the art.

In an illustrative embodiment, a portion of MEKK4 providing a Rac/Cdc42 binding site is provided in one fusion protein, along with a second fusion protein including a Rac/Cdc42 polypeptide. This embodiment of the subject assay permits the screening of compounds which inhibit or potentiate the binding of MEKK4 and Cdc42.

Phosphorylation assays may also be used. MEKK binding proteins can be tested for their ability to phosphorylate substrates in addition, compounds that inhibit or activate MEKK regulated pathways and phenotypic responses can be tested.

Furthermore, each of the assay systems set out above can be generated in a “differential” format. That is, the assay format can provide information regarding specificity as well as potency. For instance, side-by-side comparison of a test compound's effect on different MEKKs can provide information on selectivity, and permit the identification of compounds which selectively modulate the bioactivity of only a subset of the MEKK family.

The present invention also includes a method to identify compounds capable of regulating signals initiated from a receptor on the surface of a cell, such signal regulation involving in some respect, MEKK protein. Such a method comprises the steps of: (a) contacting a cell containing a MEKK protein with a putative regulatory compound; (b) contacting the cell with a ligand capable of binding to a receptor on the surface of the cell; and (c) assessing the ability of the putative regulatory compound to regulate cellular signals by determining activation of a member of a MEKK-dependent pathway of the present invention. A preferred method to perform step (c) comprises measuring the phosphorylation of a member of a MEKK-dependent pathway. Such measurements can be performed using immunoassays having antibodies specific for phosphotyrosines, phosphoserines and/or phosphothreonines. Another preferred method to perform step (c) comprises measuring the ability of the MEKK protein to phosphorylate a substrate molecule comprising a protein including MKK1, MKK2, MKK3, or MKK4, Raf-1, Ras-GAP and neurofibromin using methods described herein. Preferred substrates include MEKI, MEK2, JNKK1 and JNKK2. Yet another preferred method to perform step (c) comprises determining the ability of MEKK protein to bind to Ras, rac or Cdc 42 protein. In particular, determining the ability of MEKK protein to bind to GST-RasVl²(GTPyS) or GST-Rac^{v14}(GTPγS).

Putative compounds as referred to herein include, for example, compounds that are products of rational drug design, natural products and compounds having partially defined signal transduction regulatory properties. A putative compound can be a protein-based compound, a carbohydrate-based compound, a lipid-based compound, a nucleic acid-based compound, a natural organic compound, a synthetically derived organic compound, an anti-idiotypic antibody and/or catalytic antibody, or fragments thereof. A putative regulatory compound can be obtained, for example, from libraries of natural or synthetic compounds, in particular from chemical or combinatorial libraries (i.e., libraries of compounds that differ in sequence or size but that have the same building blocks; see for example, U.S. Pat. Nos. 5,010,175 and 5,266,684 of Rutter and Santi) or by rational drug design.

In another embodiment, a method to identify compounds capable of regulating signal transduction in a cell can comprise the steps of: (a) contacting a putative inhibitory compound with a MEKK protein to form a reaction mixture; (b) contacting the reaction mixture with MEK protein; and (c) assessing the ability of the putative inhibitory compound to inhibit phosphorylation of the MEK protein by the MEKK protein. The results obtained from step (c) can be compared with the ability of a putative inhibitory compound to inhibit the ability of Raf protein to phosphorylate MEK protein, to determine if the compound can selectively regulate signal transduction involving MEKK protein independent of Raf protein. MEKK, MEK and Raf proteins used in the foregoing methods can be recombinant proteins or naturally-derived proteins.

In another embodiment, a method to identify compounds capable of regulating signal transduction in a cell can comprise the steps of: (a) contacting a putative inhibitory compound with either a MEKK protein or a Ras superfamily protein, or functional equivalents thereof, to form a first reaction mixture; (b) combining the first reaction mixture with either Ras protein (or a functional equivalent thereof) if MEKK protein was used in the first reaction mixture, or MEKK protein (or a functional equivalent thereof) if Raf protein was added to the first reaction mixture; and (c) assessing the ability of the putative inhibitory compound to inhibit the binding of the Ras protein to the MEKK protein. The lack of binding of the MEKK protein to the Ras protein indicates that the putative inhibitory compound is effective at inhibiting binding between MEKK and Ras. MEKK and Ras proteins used in the foregoing method can be recombinant proteins or naturally-derived proteins. Preferred Ras superfamily proteins for use with the foregoing method includes, but is not limited to, GST-Ras^{V12}(GTPγS) or GST-Rac^{v14}(GTPγS).

Preferred MEKK protein for use with the method includes recombinant MEKK protein. More preferred MEKK protein includes at least a portion of a MEKK protein having the kinase domain of MEKK.

The inhibition of binding of MEKK protein to Ras superfamily protein can be determined using a variety of methods known in the art. For example, immunoprecipitation assays can be performed to determine if MEKK and Ras co-precipitate. In addition, immunoblot assays can be performed to determine if MEKK and Ras co-migrate when resolved by gel electrophoresis. Another method to determine binding of MEKK to Ras comprises combining a substrate capable of being phosphorylated by MEKK protein with the Ras protein of the reaction mixture of step (b). In this method, Ras protein is separated from the reaction mixture of step (b) following incubation with MEKK protein. If MEKK protein is able to bind to the Ras, then the bound MEKK will be co-isolated with the Ras protein. The substrate is then added to the isolated Ras protein. Any co-isolated MEKK protein will phosphorylate the substrate. Thus, inhibition of binding between MEKK and Ras can be measured by determining the extent of phosphorylation of the substrate upon combination with the isolated Ras protein. The extent of phosphorylation can be determined using a variety of methods known in the art, including kinase assays using [γ³²P]ATP. Similar assays can be performed with MEKK proteins and their binding to other GTP-binding proteins in the Ras superfamily (i.e. Rac, Cdc 42, or Rho).

Moreover, one can determine whether the site of inhibitory action along a particular signal transduction pathway involves both Raf and MEKK proteins by carrying out experiments set forth above (i.e., see discussion on MEKK-dependent pathways).

Another aspect of the present invention includes a kit to identify compounds capable of regulating signals initiated from a receptor on the surface of a cell, such signals involving in some respect, MEKK protein. Such kits include: (a) at least one cell containing MEKK protein; (b) a ligand capable of binding to a receptor on the surface of the cell; and (c) a means for assessing the ability of a putative regulatory compound to alter phosphorylation of the MEKK protein. Such a means for detecting phosphorylation include methods and reagents known to those of skill in the art, for example, phosphorylation can be detected using antibodies specific for phosphorylated amino acid residues, such as tyrosine, serine and threonine. Using such a kit, one is capable of determining, with a fair degree of specificity, the location along a signal transduction pathway of particular pathway constituents, as well as the identity of the constituents involved in such pathway, at or near the site of regulation.

In another embodiment, a kit of the present invention can include: (a) MEKK protein; (b) MEKK substrate, such as MEK; and (c) a means for assessing the ability of a putative inhibitory compound to inhibit phosphorylation of the MEKK substrate by the MEKK protein. A kit of the present invention can further comprise Raf protein and a means for detecting the ability of a putative inhibitory compound to inhibit the ability of Raf protein to phosphorylate the MEK protein.

In yet another embodiment, a mammalian MEKK gene can be used to rescue a yeast cell having a defective ste11 (or byr2) gene, such as a temperature sensitive mutant ste11 mutant (cf., Francois et al. (1991) J Biol Chem 266:6174-80; and Jenness et al. (1983) Cell 35:521-9). For example, a humanized yeast can be generated by amplifying the coding sequence of the human MEKK clone, and subcloning this sequence into a vector which contains a yeast promoter and termination sequences flanking the MEKK coding sequences. This plasmid can then be used to transform an ste11^{TS} mutant. To assay growth rates, cultures of the transformed cells can be grown at an permissive temperature for the TS mutant. Turbidity measurements, for example, can be used to easily determine the growth rate. At the non-permissive temperature, pheromone responsivenes of the yeast cells becomes dependent upon expression of the human MEKK protein. Accordingly, the humanized yeast cells can be utilized to identify compounds which inhibit the action of the human MEKK protein. It is also deemed to be within the scope of this invention that the humanized yeast cells of the present assay can be generated so as to comprise other human cell-cycle proteins. For example, human MEK and human MAPK can also be expressed in the yeast cell in place of ste7 and Fus3/Kss1. In this manner, the reagent cells of the present assay can be generated to more closely approximate the natural interactions which the mammalian MEKK protein might experience.

Furthermore, certain formats of the subject assays can be used to identify drugs which inhibit proliferation of yeast cells or other lower eukaryotes, but which have a substantially reduced effect on mammalian cells, thereby improving therapeutic index of the drug as an anti-mycotic agent. For instance, in one embodiment, the identification of such compounds is made possible by the use of differential screening assays which detect and compare drug-mediated disruption of binding between two or more different types of MEKK/MEKK-bp complexes, or which differentially inhibit the kinase activity of, for example, ste11 relative to a mammalian MEKK. Differential screening assays can be used to exploit the difference in drug-mediated disruption of human MEKK complexes and yeast ste11/byr2 complexes in order to identify agents which display a statistically significant increase in specificity for disrupting the yeast complexes (or kinase activity) relative to the human complexes. Thus, lead compounds which act specifically to inhibit proliferation of pathogens, such as fungus involved in mycotic infections, can be developed. By way of illustration, the present assays can be used to screen for agents which may ultimately be useful for inhibiting at least one fungus implicated in such mycosis as candidiasis, aspergillosis, mucormycosis, blastomycosis, geotrichosis, cryptococcosis, chromoblastomycosis, coccidioidomycosis, conidiosporosis, histoplasmosis, maduromycosis, rhinosporidosis, nocaidiosis, para-actinomycosis, penicilliosis, monoliasis, or sporotrichosis. For example, if the mycotic infection to which treatment is desired is candidiasis, the present assay can comprise comparing the relative effectiveness of a test compound on mediating disruption of a human MEKK with its effectiveness towards disrupting the equivalent ste11/byr2 kinase from genes cloned from yeast selected from the group consisting of Candida albicans, Candida stellatoidea, Candida tropicalis, Candidaparapsilosis, Candida krusei, Candida pseudotropicalis, Candida quillermondii, or Candida rugosa. Likewise, the present assay can be used to identify anti-fungal agents which may have therapeutic value in the treatment of aspergillosis by making use of genes cloned from yeast such as Aspergillus fumigatus, Aspergillus flavus, Aspergillus niger, Aspergillus nidulans, or Aspergillus terreus. Where the mycotic infection is mucormycosis, the complexes can be derived from yeast such as Rhizopus arrhizus, Rhizopus oryzae, Absidia corymbifera, Absidia ramosa, or Mucor pusillus. Sources of other ste11/byr2 homologs for comparison with a human MEKK includes the pathogen Pneumocystis carinii.

Another aspect of the present invention relates to the treatment of an animal having a medical disorder that is subject to regulation or cure by manipulating a signal transduction pathway in a cell involved in the disorder. Such medical disorders include disorders which result from abnormal cellular growth or abnormal production of secreted cellular products. In particular, such medical disorders include, but are not limited to, cancer, autoimmune disease, inflammatory responses, allergic responses and neuronal disorders, such as Parkinson's disease and Alzheimer's disease. Preferred cancers subject to treatment using a method of the present invention include, but are not limited to, small cell carcinomas, non-small cell lung carcinomas with overexpressed EGF receptors, breast cancers with overexpressed EGF or Neu receptors, tumors having overexpressed growth factor receptors of established autocrine loops and tumors having overexpressed growth factor receptors of established paracrine loops. According to the present invention, the term treatment can refer to the regulation of the progression of a medical disorder or the complete removal of a medical disorder (e.g., cure). Treatment of a medical disorder can comprise regulating the signal transduction activity of a cell in such a manner that a cell involved in the medical disorder no longer responds to extracellular stimuli (e.g., growth factors or cytokines), or the killing of a cell involved in the medical disorder through cellular apoptosis.

According to this aspect of the present invention relates to a method of inducing and/or maintaining a differentiated state, enhancing survival, and/or promoting (or alternatively inhibiting) proliferation of a cell responsive to a growth factor, morphogen or other environmental cue which effects the cell through at least one signal transduction pathway which includes a MEKK protein. In general, the method comprises contacting the cells with an amount of an agent which significantly (statistical) modulates MEKK-dependent signaling by the factor. For instance, it is contemplated by the invention that, in light of the present finding of an apparently broad involvement of members of the MEKK protein family in signal pathways implicated in the formation of ordered spatial arrangements of differentiated tissues in vertebrates, the subject method could be used to generate and/or maintain an array of different vertebrate tissue both in vitro and in vivo. A “MEKK therapeutic,” whether inductive or anti-inductive with respect to signaling by a MEKK-dependent pathway, can be, as appropriate, any of the preparations described above, including isolated polypeptides, gene therapy constructs, antisense molecules, peptidomimetics or agents identified in the drug assays provided herein.

There are a wide variety of pathological cell proliferative conditions for which MEKK therapeutics of the present invention can be used in treatment. For instance, such agents can provide therapeutic benefits where the general strategy being the inhibition of an anomalous cell proliferation. Diseases that might benefit from this methodology include, but are not limited to various cancers and leukemias, psoriasis, bone diseases, fibroproliferative disorders such as involving connective tissues, atherosclerosis and other smooth muscle proliferative disorders, as well as chronic inflammation.

In addition to proliferative disorders, the present invention contemplates the use of MEKK therapeutics for the treatment of differentiative disorders which result from, for example, de-differentiation of tissue which may (optionally) be accompanied by abortive reentry into mitosis, e.g. apoptosis. Such degenerative disorders include chronic neurodegenerative diseases of the nervous system, including Alzheimer's disease, Parkinson's disease, Huntington's chorea, amylotrophic lateral sclerosis and the like, as well as spinocerebellar degenerations. Other differentiative disorders include, for example, disorders associated with connective tissue, such as may occur due to de-differentiation of chondrocytes or osteocytes, as well as vascular disorders which involve de-differentiation of endothelial tissue and smooth muscle cells, gastric ulcers characterized by degenerative changes in glandular cells, and renal conditions marked by failure to differentiate, e.g. Wilm's tumors.

It will also be apparent that, by transient use of modulators of MEKK pathways, in vivo reformation of tissue can be accomplished, e.g. in the development and maintenance of organs. By controlling the proliferative and differentiative potential for different cells, the subject MEKK therapeutics can be used to reform injured tissue, or to improve grafting and morphology of transplanted tissue. For instance, MEKK agonists and antagonists can be employed in a differential manner to regulate different stages of organ repair after physical, chemical or pathological insult. For example, such regimens can be utilized in repair of cartilage, increasing bone density, liver repair subsequent to a partial hepatectomy, or to promote regeneration of lung tissue in the treatment of emphysema.

To further illustrate, the present method is applicable to cell culture techniques. In vitro neuronal culture systems have proved to be fundamental and indispensable tools for the study of neural development, as well as the identification of trophic and growth factors such as nerve growth factor (NGF), ciliary trophic factors (CNTF), and brain derived neurotrophic factor (BDNF). Once a neuronal cell has become terminally-differentiated it typically will not change to another terminally differentiated cell-type. However, neuronal cells can nevertheless readily lose their differentiated state. This is commonly observed when they are grown in culture from adult tissue, and when they form a blastema during regeneration. The present method provides a means for ensuring an adequately restrictive environment in order to maintain neuronal cells at various stages of differentiation, and can be employed, for instance, in cell cultures designed to test the specific activities of other trophic factors. In such embodiments of the subject method, the cultured cells can be contacted with a MEKK therapeutic in order to induce neuronal differentiation (e.g. of a stem cell), or to maintain the integrity of a culture of terminally-differentiated neuronal cells by preventing loss of differentiation. As described in PCT publication PCT/US94/11745, the default fate of ectodermal tissue is neuronal rather than mesodermal and/or epidermal. In particular, it has been reported that preventing or antagonizing signaling by activin can result in differentiation along a neuronal-fated pathway. The potential role of MEKK signaling in mesoderm induction by activin, and consequently neuronal patterning and development, is further supported by, for example, LaBonne et al. (1994) Development 120: 463-72, and LaBonne et al. (1995) Development 121: 1475-86. Accordingly, the manipulating the activities of such MAP kinases as the ERKs p38 kinases and JNKs, the subject method can be used advantagously to maintain a differentiated state, or at least to potentiate the activity of a maintenance factor such as CNTF, NGF or the like.

In an exemplary embodiment, the role of the MEKK therapeutic in the present method to culture, for example, stem cells, can be to potentiate differentiation of uncommitted progenitor cells and thereby give rise to a committed progenitor cell, or to cause further restriction of the developmental fate of a committed progenitor cell towards becoming a terminally-differentiated neuronal cell. For example, the present method can be used in vitro as part of a regimen for inducution and/or maintenance of the differentiation of neural crest cells into glial cells, schwann cells, chromaffin cells, cholinergic sympathetic or parasympathetic neurons, as well as peptidergic and serotonergic neurons. The MEKK therapeutic can be used alone, or can be used in combination with other neurotrophic factors which act to more particularly enhance a particular differentiation fate of the neuronal progenitor cell. In the later instance, a MEKK therapeutic might be viewed as ensuring that the treated cell has achieved a particular phenotypic state such that the cell is poised along a certain developmental pathway so as to be properly induced upon contact with a secondary neurotrophic factor. In similar fashion, even relatively undifferentiated stem cells or primitive neuroblasts can be maintained in culture and caused to differentiate by treatment with MEKK therapeutics. Exemplary primitive cell cultures comprise cells harvested from the neural plate or neural tube of an embryo even before much overt differentiation has occurred.

Yet another aspect of the present invention concerns the application of MEKK therapeutics to modulating morphogenic signals involved in other vertebrate organogenic pathways in addition to neuronal differentiation. Thus, it is contemplated by the invention that compositions comprising MEKK therapeutics can also be utilized for both cell culture and therapeutic methods involving generation and maintenance of non-neuronal tissue.

In one embodiment, the present invention makes use of the notion that MEKK proteins are likely to be involved in controlling the development and formation of the digestive tract, liver, pancreas, lungs, and other organs which derive from the primitive gut. As described in the Examples below, MEKK proteins are presumptively involved in cellular activity in response to inductive signals. Additionally, it has been demonstrated that the activity of a JNK enzyme is markedly stimulated during regeneration after partial hepatectomy, with a concomitant increase in phosphorylated c-Jun. Accordingly, MEKK agonists and/or antagonists can be employed in the development and maintenance of an artificial liver which can have multiple metabolic functions of a normal liver. In an exemplary embodiment, MEKK therapeutics can be used to induce and/or maintain differentiation of digestive tube stem cells to form hepatocyte cultures which can be used to populate extracellular matrices, or which can be encapsulated in biocompatible polymers, to form both implantable and extracorporeal artificial livers.

In another embodiment, compositions of MEKK therapeutics can be utilized in conjunction with transplantation of such artificial livers, as well as embryonic liver structures, to promote intraperitoneal implantation, vascularization, and in vivo differentiation and maintenance of the engrafted liver tissue.

Similar utilization of MEKK therapeutics are contemplated in the generation and maintenance of pancreatic cultures and artificial pancreatic tissues and organs.

In another embodiment, in vitro cell cultures can be used for the identification, isolation, and study of genes and gene products that are expressed in response to disruption of MEKK-mediated signal transduction, and therefore likely involved in development and/or maintenance of tissues. These genes would be “downstream” of the MEKK gene products. For example, if new transcription is required for a MEKK-mediated induction, a subtractive cDNA library prepared with control cells and cells overexpressing a MEKK gene can be used to isolate genes that are turned on or turned off by this process. The powerful subtractive library methodology incorporating PCR technology described by Wang and Brown is an example of a methodology useful in conjunction with the present invention to isolate such genes (Wang et al. (1991) PNAS 88:11505-11509). Utilizing control and treated cells, the induced pool can be subtracted from the uninduced pool to isolate genes that are turned on, and then the uninduced pool from the induced pool for genes that are turned off. From this screen, it is expected that two classes of mRNAs can be identified. Class I RNAs would include those RNAs expressed in untreated cells and reduced or eliminated in induced cells, that is the down-regulated population of RNAs. Class II RNAs include RNAs that are upregulated in response to induction and thus more abundant in treated than in untreated cells. RNA extracted from treated vs untreated cells can be used as a primary test for the classification of the clones isolated from the libraries.

In still another embodiment of the present invention, compositions comprising MEKK therapeutics can be used for the in vitro generation of skeletal tissue, such as from skeletogenic stem cells, as well as for the in vivo treatment of skeletal tissue deficiencies. The present invention contemplates the use of MEKK therapeutics which upregulate or mimic the inductive activity of a bone morphogenetic protein (BMP) or TGF-β, such as may be useful to control chondrogenesis and/or osteogenesis. By “skeletal tissue deficiency”, it is meant a deficiency in bone or other skeletal connective tissue at any site where it is desired to restore the bone or connective tissue, no matter how the deficiency originated, e.g. whether as a result of surgical intervention, removal of tumor, ulceration, implant, fracture, or other traumatic or degenerative conditions, so long as modulation of a TGF-β inductive response is appropriate.

For instance, the present invention makes available effective therapeutic methods and MEKK therapeutic compositions for restoring cartilage function to a connective tissue. Such methods are useful in, for example, the repair of defects or lesions in cartilage tissue which is the result of degenerative wear such as that which results in arthritis, as well as other mechanical derangements which may be caused by trauma to the tissue, such as a displacement of torn meniscus tissue, meniscectomy, a Taxation of ajoint by a torn ligament, malignment of joints, bone fracture, or by hereditary disease. The present reparative method is also useful for remodeling cartilage matrix, such as in plastic or reconstructive surgery, as well as periodontal surgery. The present method may also be applied to improving a previous reparative procedure, for example, following surgical repair of a meniscus, ligament, or cartilage. Furthermore, it may prevent the onset or exacerbation of degenerative disease if applied early enough after trauma.

The present invention further contemplates the use of the subject method in the field of cartilage transplantation and prosthetic device therapies. To date, the growth of new cartilage from either transplantation of autologous or allogenic cartilage has been largely unsuccessful. Problems arise, for instance, because the characteristics of cartilage and fibrocartilage varies between different tissue: such as between articular, meniscal cartilage, ligaments, and tendons, between the two ends of the same ligament or tendon, and between the superficial and deep parts of the tissue. The zonal arrangement of these tissues may reflect a gradual change in mechanical properties, and failure occurs when implanted tissue, which has not differentiated under those conditions, lacks the ability to appropriately respond. For instance, when meniscal cartilage is used to repair anterior cruciate ligaments, the tissue undergoes a metaplasia to pure fibrous tissue. By helping to control chondrogenesis, the subject method can be used to particularly addresses this problem, by causing the implanted cells to become more adaptive to the new environment and effectively resemble hypertrophic chondrocytes of an earlier developmental stage of the tissue. Thus, the action of chondrogensis in the implanted tissue, as provided by the subject method, and the mechanical forces on the actively remodeling tissue can synergize to produce an improved implant more suitable for the new function to which it is to be put.

In similar fashion, the subject method can be applied to enhancing both the generation of prosthetic cartilage devices and to their implantation. In one embodiment of the subject method, the implants are contacted with a MEKK therapeutic during the culturing process so as to induce and/or maintain differentiated chondrocytes in the culture in order to further stimulate cartilage matrix production within the implant. In such a manner, the cultured cells can be caused to maintain a phenotype typical of a chondrogenic cell (i.e. hypertrophic), and hence continue the population of the matrix and production of cartilage tissue.

In another embodiment, the implanted device is treated with a MEKK therapeutic in order to actively remodel the implanted matrix and to make it more suitable for its intended function. As set out above with respect to tissue transplants, the artificial transplants suffer from the same deficiency of not being derived in a setting which is comparable to the actual mechanical environment in which the matrix is implanted. The activation of the chondrocytes in the matrix by the subject method can allow the implant to acquire characteristics similar to the tissue for which it is intended to replace.

In yet another embodiment, the subject method is used to enhance attachment of prosthetic devices. To illustrate, the subject method can be used in the implantation of a periodontal prosthesis, wherein the treatment of the surrounding connective tissue stimulates formation of periodontal ligament about the prosthesis, as well as inhibits formation of fibrotic tissue proximate the prosthetic device.

In still further embodiments, the subject method can be employed for the generation of bone (osteogenesis) at a site in the animal where such skeletal tissue is deficient. A variety of factors which may signal through MEKK proteins are associated with the hypertrophic chondrocytes that are ultimately replaced by osteoblasts as well as the production of bone matrix by osteocytes. Consequently, administration of a MEKK therapeutic can be employed as part of a method for treating bone loss in a subject, e.g. to prevent and/or reverse osteoporosis and other osteopenic disorders, as well as to regulate bone growth and maturation. For example, preparations comprising MEKK therapeutics can be employed, for example, to induce endochondral ossification by mimicking or potentiating the activity of a BMP, at least so far as to facilitate the formation of cartilaginous tissue precursors to form the “model” for ossification. Therapeutic compositions of such MEKK therapeutics can be supplemented, if required, with other osteoinductive factors, such as bone growth factors (e.g. TGF-β factors, such as the bone morphogenetic factors BMP-2 and BMP-4, as well as activin), and may also include, or be administered in combination with, an inhibitor of bone resorption such as estrogen, bisphosphonate, sodium fluoride, calcitonin, or tamoxifen, or related compounds.

In yet another embodiment, treatment with a MEKK therapeutic may permit disruption of autocrine loops, such as PDGF autostimulatory loops, believed to be involved in the neoplastic transformation of several neuronal tumors. Modulation of certain of the MEKK proteins may, therefore, be of use to either prevent de-differentiation into mitotic phenotype, or even to induce apoptosis in such cells. Accordingly, the subject MEKK therapeutics may be useful in the treatment of, for example, malignant gliomas, medulloblastomas, neuroectodermal tumors, and ependymonas.

For certain cell-types, particularly in epithelial and hemopoietic cells, normal cell proliferation is marked by responsiveness to negative autocrine or paracrine growth regulators. This is generally accompanied by differentiation of the cell to a post-mitotic phenotype. However, it has been observed that a significant percentage of human cancers derived from these cells types display a reduced responsiveness to growth regulators such as TGFβ. For instance, some tumors of colorectal, liver epithelial, and epidermal origin show reduced sensitivity and resistance to the growth-inhibitory effects of TGFβ as compared to their normal counterparts. Treatment of such tumors with MEKK therapeutics provides an opportunity to mimic the effective function of TGFβ-mediated inhibition by constitutive activation of that pathway, and/or offset other competing pathways which become dominant upon lose of TGFβ responsiveness.

To further illustrate the use of the subject method, the therapeutic application of a MEKK therapeutic can be used in the treatment of a neuroglioma. Gliomas account for 40-50% of intracranial tumors at all ages of life. Despite the increasing use of radiotherapy, chemotherapy, and sometimes immunotherapy after surgery for malignant glioma, the mortality and morbidity rates have not substantially improved. However, there is increasing experimental and clinical evidence that for a significant number of gliomas, loss of TGFβ responsiveness is an important event in the loss of growth control. Where the cause of decreased responsivenessis due to loss of receptor or loss of other TGFβ signal transduction downstream of the receptor, treatment with a MEKK therapeutic can be used to constitutively activate the TGFβ pathway and restore growth inhibition. Alternatively, by manipulation of the level activation of the ERKs, apoptosis may be induced.

The subject MEKK therapeutics can also be used in the treatment of hyperproliferative vascular disorders, e.g. smooth muscle hyperplasia (such as atherosclerosis) or restinosis, as well as other disorders characterized by fibrosis, e.g. rheumatoid arthritis, insulin dependent diabetes mellitus, glomerulonephritis, cirrhosis, and scleroderma, particularly proliferative disorders in which aberrant autocrine or paracrine signaling is implicated.

For example, restinosis continues to limit the efficacy of coronary angioplasty despite various mechanical and pharmaceutical interventions that have been employed. An important mechanism involved in normal control of intimal proliferation of smooth muscle cells appears to be the induction of autocrine and paracrine TGFβ inhibitory loops in the smooth muscle cells (Scott-Burden et al. (1994) Tex Heart Inst J 21:91-97; Graiger et al. (1993) Cardiovasc Res 27:2238-2247; and Grainger et al. (1993) Biochem J294:109-112). Loss of sensitivity to TGFβ, or alternatively, the overriding of this inhibitory stimulus such as by PDGF autostimulation, can be a contributory factor to abnormal smooth muscle proliferation in restinosis. It may therefore be possible to treat or prevent restinosis by the use of MEKK therapapeutics which mimic or restore induction by TGFβ or which inhibit PDGF stimulation.

Aberrant signaling by both positive and negative growth regulators also play a significant role in local glomerular and interstitial sites in human kidney development and disease. Consequently, the subject method provides a method of treating or inhibiting glomerulopathies and other renal proliferative disorders comprising the in vivo delivery of a subject MEKK therapeutic.

Yet another aspect of the present invention concerns the therapeutic application of a MEKK therapeutic to enhance survival of neurons and other neuronal cells in both the central nervous system and the peripheral nervous system. The ability of signals transduced through MEKK proteins to regulate neuronal differentiation and survival indicates that certain of the MEKK proteins can be reasonably expected to participate in control of adult neurons with regard to maintenance, functional performance, and aging of normal cells; repair and regeneration processes in chemically or mechanically lesioned cells; and prevention of degeneration and premature death which result from loss of differentiation in certain pathological conditions. In light of this understanding, the present invention specifically contemplates applications of the subject method to the treatment of (prevention and/or reduction of the severity of) neurological conditions deriving from: (i) acute, subacute, or chronic injury to the nervous system, including traumatic injury, chemical injury, vasal injury and deficits (such as the ischemia resulting from stroke), together with infectious/ inflammatory and tumor-induced injury; (ii) aging of the nervous system including Alzheimer's disease; (iii) chronic neurodegenerative diseases of the nervous system, including Parkinson's disease, Huntington's chorea, amylotrophic lateral sclerosis and the like, as well as spinocerebellar degenerations; and (iv) chronic immunological diseases of the nervous system or affecting the nervous system, including multiple sclerosis.

Many neurological disorders are associated with degeneration of discrete populations of neuronal elements and may be treatable with a therapeutic regimen which includes a MEKK therapeutic. For example, Alzheimer's disease is associated with deficits in several neurotransmitter systems, both those that project to the neocortex and those that reside with the cortex. For instance, the nucleus basalis in patients with Alzheimer's disease have been observed to have a profound (75%) loss of neurons compared to age-matched controls. Although Alzheimer's disease is by far the most common form of dementia, several other disorders can produce dementia. Several of these are degenerative diseases characterized by the death of neurons in various parts of the central nervous system, especially the cerebral cortex. However, some forms of dementia are associated with degeneration of the thalmus or the white matter underlying the cerebral cortex. Here, the cognitive dysfunction results from the isolation of cortical areas by the degeneration of efferents and afferents. Huntington's disease involves the degeneration of intrastraital and cortical cholinergic neurons and GABAergic neurons. Pick's disease is a severe neuronal degeneration in the neocortex of the frontal and anterior temporal lobes, sometimes accompanied by death of neurons in the striatum. Treatment of patients suffering from such degenerative conditions can include the application of MEKK therapeutics, in order to control, for example, differentiation and apoptotic events which give rise to loss of neurons (e.g. to enhance survival of existing neurons) as well as promote differentiation and repopulation by progenitor cells in the area affected.

In addition to degenerative-induced dementias, a pharmaceutical preparation of one or more of the subject MEKK therapeutics can be applied opportunely in the treatment of neurodegenerative disorders which have manifestations of tremors and involuntary movements. Parkinson's disease, for example, primarily affects subcortical structures and is characterized by degeneration of the nigrostriatal pathway, raphe nuclei, locus cereleus, and the motor nucleus of vagus. Ballism is typically associated with damage to the subthalmic nucleus, often due to acute vascular accident.

Also included are neurogenic and myopathic diseases which ultimately affect the somatic division of the peripheral nervous system and are manifest as neuromuscular disorders. In an illustrative embodiment, the subject method is used to treat amyotrophic lateral sclerosis. ALS is a name given to a complex of disorders that comprise upper and lower motor neurons. Patients may present with progressive spinal muscular atrophy, progressive bulbar palsy, primary lateral sclerosis, or a combination of these conditions. The major pathological abnormality is characterized by a selective and progressive degeneration of the lower motor neurons in the spinal cord and the upper motor neurons in the cerebral cortex. The therapeutic application of a MEKK therapeutic, can be used alone, or in conjunction with neurotrophic factors such as CNTF, BDNF or NGF to prevent and/or reverse motor neuron degeneration in ALS patients.

MEKK therapeutics can also be used in the treatment of autonomic disorders of the peripheral nervous system, which include disorders affecting the innervation of smooth muscle and endocrine tissue (such as glandular tissue). For instance, the subject method can be used to treat tachycardia or atrial cardiac arrythmias which may arise from a degenerative condition of the nerves innervating the striated muscle of the heart.

In yet another embodiment, modulation of a MEKK-dependent pathway can be used to inhibit spermatogenesis. Spermatogenesis is a process involving mitotic replication of a pool of diploid stem cells, followed by meiosis and terminal differentiation of haploid cells into morphologically and functionally polarized spermatoza. This process exhibits both temporal and spatial regulation, as well as coordinated interaction between the germ and somatic cells. It has been previously shown that the signals coupling extracellular stimulus to regulation of mitotic, meiotic events which occur during spermatogenesis include pathways which rely on, for example, MAP kinases, for propagation. Accordingly, certain of these pathways may include MEKK proteins and be alterable by the subject MEKK therapeutics.

Likewise, members of the MAPK proteins are important in the regulation of female reproductive organs (Wu, T. C. et al. (1994) Mol. Reprod. Dev. 38:9-15). Accordingly, certain of the MEKK therapeutics may be useful to prevent oocyte maturation as part of a contraceptive formulation. In other aspects, regulation of induction of meiotic maturation with MEKK therapeutics can be used to synchronize oocyte populations for in vitro fertilization. Such a protocol can be used to provide a more homogeneous population of oocytes which are healthier and more viable and more prone to cleavage, fertilization and development to blastocyst stage. In addition the MEKK therapeutics could be used to treat other disorders of the female reproductive system which lead to infertility including polycysitic ovarian syndrome.

Another aspect of the invention features transgenic non-human animals which express a heterologous MEKK gene of the present invention, or which have had one or more genomic MEKK genes disrupted in at least one of the tissue or cell-types of the animal. Accordingly, the invention features an animal model for developmental diseases, which animal has MEKK allele which is mis-expressed. For example, a mouse can be bred which has one or more MEKK alleles deleted or otherwise rendered inactive. Such a mouse model can then be used to study disorders arising from mis-expressed MEKK genes, as well as for evaluating potential therapies for similar disorders.

Another aspect of the present invention concerns transgenic animals which are comprised of cells (of that animal) which contain a transgene of the present invention and which preferably (though optionally) express an exogenous MEKK protein in one or more cells in the animal. A MEKK transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a MEKK protein can be essential for many forms of lineage analysis and can additionally provide a means to assess the effects of, for example, lack of MEKK expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific genetic manipulation in vivo are known to those skilled in the art. For instance, genetic svst ;ms are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject MEKK proteins. For example, excision of a target sequence which interferes with the expression of a recombinant MEKK gene, such as one which encodes an antagonistic homolog or an antisense transcript, can be designed to activate expression of that gene. This interference with expression of the protein can result from a variety of mechanisms, such as spatial separation of the MEKK gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant MEKK protein can be regulated via control of recombinase expression.

Use of the crelloxP recombinase system to regulate expression of a recombinant MEKK protein requires the construction of a transgenic animal containing transgenes encoding both the Cre recombinase and the subiect protein. Animals containing both the Cre recombinase and a recombinant MEKK gene can be provided through the construction of “double” transgenic animals. A convenient method for providing such animals is to mate two transgenic animals each containing a transgene, e.g., a MEKK gene and recombinase gene.

One advantage derived from initially constructing transgenic animals containing a MEKK transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic MEKK transgene is silent will allow the study of progeny from that founder in which disruption of MEKK mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the MEKK transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, a MEKK transgene could remain silent into adulthood until “turned on” by the introduction of the trans-activator.

In an exemplary embodiment, the “transgenic non-human animals” of the invention are produced by introducing transgenes into the germline of the non-human animal. Embryonic target cells at various developmental stages can be used to introduce transgenes. Different methods are used depending on the stage of development of the embryonic target cell. The zygote is the best target for micro-injection. In the mouse, the male pronucleus reaches the size of approximately 20 micrometers in diameter which allows reproducible injection of 1-2pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic non-human animal will carry the incorporated transgene. This will in general also be reflected in the efficient transmission of the transgene to offspring of the founder since 50% of the germ cells will harbor the transgene. Microinjection of zygotes is the preferred method for incorporating transgenes in practicing the invention.

Retroviral infection can also be used to introduce MEKK transgenes into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonic stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Methods of making MEKK knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert recombinase target sequences flanking portions of an endogenous MEKK gene, such that tissue specific and/or temporal control of inactivation of a MEKK allele can be controlled as above.

One aspect of the present invention involves the recognition that a MEKK protein of the present invention is capable of regulating the homeostas;s of a cell by regulating cellular activity such as cell growth cell death, and cell function (e.g., secretion of cellular products). Such regulation, in most cases, is independent of Raf, however, as discussed above (and as shown in FIG. 2), some pathways capable of regulation by MEKK protein may be subject to upstream regulation by Raf protein. Therefore, it is within the scope of the present invention to either stimulate or inhibit the activity of Raf protein and/or MEKK protein to achieve desired regulatory results. Without being bound by theory, it is believed that the regulation of Raf protein and MEKK protein activity at the divergence point from Ras protein (see FIG. 2) can be controlled by a “2-hit” mechanism. For example, a first “hit” can comprise any means of stimulating Ras protein, thereby stimulating a Ras-dependent pathway, including, for example, contacting a cell with a growth factor which is capable of binding to a cell surface receptor in such a manner that Ras protein is activated. Following activation of Ras protein, a second “hit” can be delivered that is capable of increasing the activity of JNK activity compared with MAPK activity, or vice versa. A second “hit” can include, but is not limited to, regulation of JNK or MAPK activity by compounds capable of stimulating or inhibiting the activity of MEKK, JNKK (MKK3 or MKK4), Raf and/or MEK. For example, compounds such as protein kinase C or phospholipase C kinase, can provide the second “hit” needed to drive the divergent Ras-dependent pathway down the MEKK-dependent pathway in such a manner that JNK is preferentially activated over MAPK.

One embodiment of the present invention comprises a method for regulating the homeostasis of a cell comprising regulating the activity of a MEKK-dependent pathway relative to the activity of a Raf-dependent pathway in the cell. As used herein, the term “homeostasis” refers to the tendency of a cell to maintain a normal state using intracellular systems such as signal transduction pathways. Regulation of the activity of a MEKK-dependent pathway includes increasing the activity of a MEKK-dependent pathway relative to the activity of a Raf-dependent pathway by regulating the activity of a member of a MEKK-dependent pathway, a member of a Raf-dependent pathway, and combinations thereof, to achieve desired regulation of phosphorylation along a given pathway, and thus effect apoptosis. Preferred regulated members of a MEKK-dependent pathway or a Raf-dependent pathway to regulate include, but are not limited to, proteins including MEKK, Ras, Rac, Cdc 42, Raf, MKK, JNKK, MEK, MAPK, JNK, TCF, ATF-2, Jun and Myc, and combinations thereof.

In one embodiment, the activity of a member of a MEKK-dependent pathway, a member of a Raf-dependent pathway, and combinations thereof, are regulated by altering the concentration of such members in a cell. One preferred regulation scheme involves altering the concentration of proteins including MEKK, Ras, Rac, Cdc 42, Raf, JNKK, MEK, MAPK, JNK, TCF, Jun, ATF-2, and Myc, and combinations thereof. A more preferred regulation scheme involves increasing the concentration of proteins including MEKK, Ras, Rac, Cdc 42, JNKK, JNK, Jun, ATF-2, and Myc, and combinations thereof. Another more preferred regulation scheme involves decreasing the concentration of proteins including Raf, MEK, MAPK, and TCF, and combinations thereof. It is also within the scope of the present invention that the regulation of protein concentrations in two or more of the foregoing regulation schemes can be combined to achieve an optimal apoptotic effect in a cell.

A preferred method for increasing the concentration of a protein in a regulation scheme of the present invention includes, but is not limited to, increasing the copy number of a nucleic acid sequence encoding such protein within a cell, improving the efficiency with which the nucleic acid sequence encoding such protein is transcribed within a cell, improving the efficiency with which a transcript is translated into such a protein, improving the efficiency of post-translational modification of such protein, contacting cells capable of producing such protein with anti-sense nucleic acid sequences, and combinations thereof.

In a preferred embodiment of the present invention, the homeostasis of a cell is controlled by regulating the apoptosis of a cell. A suitable method for regulating the apoptosis of a cell is to regulate the activity of a MEKK-dependent pathway in which the MEKK protein regulates the pathway substantially independent of Raf. A particularly preferred method for regulating the apoptosis of a cell comprises increasing the concentration of MEKK protein by contacting a cell with a nucleic acid molecule encoding a MEKK protein that possesses unregulated kinase activity.

It is within the scope of the invention that the foregoing method can further comprise the step of decreasing the activity of MEK protein in the cell by contacting the cell with a compound capable of inhibiting MEK activity. Such compounds can include: peptides capable of binding to the kinase domain of MEK in such a manner that phosphorylation of MAPK protein by the MEK protein is inhibited; and/or peptides capable of binding to a portion of a MAPK protein in such a manner that phosphorylation of the MAPK protein is inhibited.

In another embodiment, the activity of a member of a MEKK-dependent pathway, a member of a Raf-dependent pathway, and combinations thereof, can be regulated by directly altering the activity of such members in a cell. A preferred method for altering the activity of a member of a MEKK-dependent pathway, includes, but is not limited to, contacting a cell with a compound capable of directly interacting with a protein including MEKK, Ras, Rac, Cdc 42, JNKK, JNK, Jun, ATF-2, and Myc, and combinations thereof, in such a manner that the proteins are activated; and/or contacting a cell with a compound capable of directly interacting with a protein including Raf, MEK, MAPK, TCF protein, and combinations thereof in such a manner that the activity of the proteins are inhibited. A preferred compound with which to contact a cell that is capable of regulating a member of a MEKK-dependent pathway includes a peptide capable of binding to the regulatory domain of proteins including MEKK, Ras, Rac, Cdc 42, JNKK, JNK, Jun, ATF-2, and Myc, in which the peptide inhibits the ability of the regulatory domain to regulate the activity of the kinase domains of such proteins. Another preferred compound with which to contact a cell includes TNFα, growth factors regulating tyrosine kinases, hormones regulating G protein-coupled receptors and FAS ligand.

A preferred compound with which to contact a cell that is capable of regulating a member of a Raf-dependent pathway includes a peptide capable of binding to the kinase catalytic domain of a protein selected from the group consisting of Raf, MEK-1, MEK-2, MAPK, and TCF, in which the peptide inhibits the ability of the protein to be phosphorylated or to phosphorylate a substrate.

In accordance with the present invention, a compound can regulate the activity of a member of a MEKK-dependent pathway by affecting the ability of one member of the pathway to bind to another member of the pathway. Inhibition of binding can be achieved by directly interfering at the binding site of either member, or altering the conformational structure, thereby precluding the binding between one member and another member.

Another preferred compound with which to contact a cell that is capable of regulating a member of a MEKK-dependent pathway includes an isolated compound that is capable of regulating the binding of MEKK protein to a protein of the Ras superfamily, such as Ras, Rac, Cdc 42, or Rho (referred to herein as a Ras:MEKK binding compound). In one embodiment, a Ras:MEKK binding compound of the present invention comprises an isolated peptide (or mimetope thereof) comprising an amino acid sequence derived from a Ras superfamily protein. In another embodiment, a Ras:MEKK binding compound of the present invention comprises an isolated peptide (or mimetope thereof) comprising an amino acid sequence derived from a MEKK protein.

According to the present invention, an isolated, or biologically pure, peptide, is a peptide that has been removed from its natural milieu. As such, “isolated” and “biologically pure” do not necessarily reflect the extent to which the protein has been purified. An isolated compound of the present invention can be obtained from a natural source or produced using recombinant DNA technology or chemical synthesis. As used herein, an isolated peptide can be a full-length protein or any homolog of such a protein in which amino acids have been deleted (e.g., a truncated version of the protein), inserted, inverted, substituted and/or derivatized (e.g., by glycosylation, phosphorylation, acetylation, myristylation, prenylation, palmitilation, and/or amidation) such that the peptide is capable of regulating the binding of Ras superfamily protein to MEKK protein.

In accordance with the present invention, a “mimetope” refers to any compound that is able to mimic the ability of an isolated compound of the present invention. A rlimetope can be a peptide that has been modified to decrease its susceptibility to degradation but that still retain regulatory activity. Other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds as disclosed herein that are capable of inhibiting the binding of Ras superfamily protein to MEKK. A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. The predicted mimetope structures can then be produced by, for example, chemical synthesis, recombinant DNA technology, or by isolating a mimetope from a natural source (e.g., plants, animals, bacteria and fungi).

In one embodiment, a Ras:MEKK binding compound of the present invention comprises an isolated peptide having a domain of a Ras superfamily protein that is capable of binding to a MEKK protein (i.e., that has an amino acid sequence which enables the peptide to be bound by a MEKK protein). A Ras peptide of the present invention is of a size that enables the peptide to be bound by a MEKK protein, preferably, at least about 4 amino acid residues, more preferably at least about 12 amino acid residues, and even more preferably at least about 25 amino acid residues. In particular, a Ras peptide of the present invention is capable of being bound by the COOH-terminal region of MEKK, in certain embodiments the region of MEKK containing the MEKK kinase domain. Preferably, a Ras peptide of the present invention comprises the effector domain of Ras and more preferably amino acid residues 17-42 of H-Ras. In addition, similar domains of Rac are important for the binding of Rac, Cdc 42 or Rho to certain MEKK proteins.

In another embodiment, a Ras:MEKK binding compound of the present invention comprises an isolated MEKK peptide that has a domain of a MEKK protein that is capable of binding to a Ras protein (i.e., that has an amino acid sequence which enables the peptide to be bound by a Ras protein). A MEKK peptide of the present invention is of a size that enables the peptide to be bound by a Ras protein, in particular by the effector domain of a Ras protein. Preferably, a MEKK peptide of the present invention at least about 320 amino acids in length. Preferably, a MEKK peptide of the present invention comprises the COOH-terminal region of a MEKK protein and more preferably MEKKCooH (as described in detail in the appended examples).

In one embodiment the Rac-binding portior of a MEKK protein or a fragment thereof is used to block the binding of the MEKK catalytic domain with Cdc42 and Rac, thus inhibiting MEKK activity. Preferred fragment lengths are at least about 4 amino acids, preferably about 8 amino acids, more preferably about 12 amino acids, although longer framents are also contemplated. Similarly the consensus PAK sequence or fragments thereof could be used to block the binding of MEKK and Cdc42 or Rac. In another embodiment peptidomimetics or mimetopes of these fragments are used. In another embodiment a Ras effector domain peptide is used to blocks the binding of the MEKK catalytic domain with the GTP-bound form of Ras. Alternatively, the portion of the MEKK catalytic domain which binds to Ras, or the Ras effector domain can be used to competitively inhibit binding of Ras and a MEKK protein.

Ras is a critical component of tyrosine kinase growth factor receptor and G-protein coupled receptor regulation of signal transduction pathways controlling mitogenesis and differentiation. According to the present invention, the protein serine-threonine kinases Raf-1 and MEKK1 are Ras effectors and selectively bind to Ras in a GTP dependent manner. The p110 catalytic subunit of the lipid kinase has also been shown to directly interact with Ras in a GTP dependent manner. Ras-GAP and neurofibromin also regulate Ras GTPase activity. Raf-1, MEKK1 and P13-kinase are capable of increasing the activity in cells expressing GTPase-deficient Ras consistent with their interaction with Ras-GTP being involved in their regulation.

Different functional domains of Ras effectors bind to Ras in a GTP dependent manner. The Ras binding domain for Raf-1 is encoded in the extreme NH₂-terminal regulatory domain of Raf-1. The Ras binding domain is encoded within the catalytic domain of MEKK1. Both Raf-1 and MEKK1 binding to Ras is blocked by a Ras effector domain peptide. Thus, Raf-1, MEKK1 and other Ras effectors can compete for interaction with Ras-GTP presumably at the Ras effector domain. The relative abundance and affinity of each Ras effector in different cells may influence the magnitude, onset and duration of each effector response. Secondary inputs, such as phosphorylation of the different Ras effectors, can also influence their interaction with Ras-GTP. The kinetic properties of Ras effector activation in cells relative to effector affinity for Ras-GTP are predictable based on the foregoing information. For example, MEKK1 can preferentially regulate the SEK/Jun kinase pathways relative to MAPK. Activation of the SEK/Jun kinase pathway is generally slower in onset and maintained as maximal activity longer than the activation of MAPK.

As additional MEKKs are characterized it will be important to characterize their regulation and interaction with other members of the Ras superfamily. For example, MEKK4.1 and 4.2 have been found to bind to Rac/Cdc42 as described herein. Rho, Rac, and Cdc42 are small GTPases that have been implicated in the formation of a variety of actin structures and the assembly of associated integrin complexes (Burbelo, et al. (1995) J. Biol Chem. 270:29071-29074). One of the targets of the Cdc42 and Rac GTPases is the PAK family of protein kinases (Bagrodia et al (1995) J. Biol. Chem 270:27995-27998). Rac and Cdc42 have been shown to regulate the activity of the JNK/SAPK signaling pathway in ways different from Ras. While activated Ras stimulates MAPK, but poorly induces JNK activity, mutationally activated Rac 1 and Cdc42 GTPases potently activate JNK without affecting MAPK (Coso et al. (1995) Cell 81:1137-1 146). Undoubtedly additional Ras effectors which interact with and regulate MEKK proteins, perhaps resulting in the selective activation of certain substrates, will be identified in the near future. The present invention also includes a method to administer isolated compounds of the present invention to a cell to regulate signal transduction activity in the cell. In particular, the present invention includes a method to administer an isolated compound of the present invention to a cell to regulate apoptosis of the cell.

Compounds of the present invention may influence cellular mitogenesis, DNA synthesis, cell division and differentiation. MAPK is also recognized as being involved in the activation of oncogenes, such as c-jun and c-myc. While not bound by theory, the present inventor believes that MAPK is also intimately involved in various abnormalities having a genetic origin. MAPK is known to cross the nuclear membrane and is believed to be at least partially responsible for regulating the expression of various genes. As such, MAPK is believed to play a significant role in the instigation or progression of cancer, neuronal diseases, autoimmune diseases, allergic reactions, wound healing and inflammatory responses. The present inventor, by being first to identify nucleic acid sequences encoding MEKK, recognized that it is now possible to regulate the expression of MEKK, and thus regulate the activation of MAPK.

The present invention also includes a method for regulating the homeostasis of a cell comprising injecting an area of a subject's body with an effective amount of a naked plasmid DNA compound (such as is taught, for example in Wolff et al., 1990, Science 247, 1465-1468). A naked plasmid DNA compound comprises a nucleic acid molecule encoding a MEKK protein of the present invention, operatively linked to a naked plasmid DNA vector capable of being taken up by and expressed in a recipient cell located in the body area. A preferred naked plasmid DNA compound of the present invention comprises a nucleic acid molecule encoding a truncated MEKK protein having deregulated kinase activity. Preferred naked plasmid DNA vectors of the present invention include those known in the art. When administered to a subject, a naked plasmid DNA compound of the present invention transforms cells within the subject and directs the production of at least a portion of a MEKK protein or RNA nucleic acid molecule that is capable of regulating the apoptosis of the cell.

A naked plasmid DNA compound of the present invention is capable of treating a subject suffering from a medical disorder including cancer, autoimmune disease, inflammatory responses, allergic responses and neuronal disorders, such as Parkinson's disease and Alzheimer's disease. For example, a naked plasmid DNA compound can be administered as an anti-tumor therapy by injecting an effective amount of the plasmid directly into a tumor so that the plasmid is taken up and expressed by a tumor cell, thereby killing the tumor cell. As used herein, an effective amount of a naked plasmid DNA to administer to a subject comprises an amount needed to regulate or cure a medical disorder the naked plasmid DNA is intended to treat, such mode of administration, number of doses and frequency of dose capable of being decided upon, in any given situation, by one of skill in the art without resorting to undue experimentation.

One aspect of the present invention relates to the recognition that a MEKK protein is capable of activating MAPK and that MAPK can regulate various cellular functions as disclosed in U.S. Pat. No. 5,405,941, which is incorporated herein by this reference.

One example of a therapeutic compound of the present invention is the nucleic acid encoding the amino acid residues 1306-1326 of MEKK4.2 or 599-619 of MEKK4. In other embodiments the peptide or fragments thereof can be used. The Cdc42/Rac binding region of a MEKK peptide (IIGQVCDTPKSYDNVMHVGLR) or the nucleic acid which encodes it can be used to inhibit the binding of MEKK and a member of the Ras superfamily. Alternatively, the domain of Rac or Cdc42 to which it binds could be used. In another embodiment the region of the Ras effector domain which blocks the binding of the MEKK catalytic domain with the GTP-bound form of Ras could be used. Alternatively, the portion of the MEKK catalytic domain which binds to Ras could be used to block MEKK-Ras interaction.

An isolated compound of the present invention can be used to formulate a therapeutic composition. In one embodiment, a therapeutic composition of the present invention includes at least one isolated peptide of the present invention. A therapeutic composition for use with a treatment method of the present invention can further comprise suitable excipients. A therapeutic compound for use with a treatment method of the present invention can be formulated in an excipient that the subject to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful excipients include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taker up in a suitable liquid as a suspension or solution for injection. Thus, in a non-liquid formulation, the excipient can comprise dextrose, human serum albumin, preservatives, etc., to which sterile water or saline can be added prior to administration.

In another embodiment, a therapeutic compound for use with a treatment method of the present invention can also comprise a carrier. Carriers are typically compounds that increase the half-life of a therapeutic compound in the treated animal. Suitable carriers include, but are not limited to, liposomes, micelles, cells, polymeric controlled release formulations, biodegradable implants, bacteria, viruses, oils, esters, and glycols. Preferred carriers include liposomes and micelles.

A therapeutic compound for use with a treatment method of the present invention can be administered to any subject having a medical disorder as herein described. Acceptable protocols by which to administer therapeutic compounds of the present invention in an effective manner can vary according to individual dose size, number of doses, frequency of dose administration, and mode of administration. Determination of such protocols can be accomplished by those skilled in the art without resorting to undue experimentation. An effective dose refers to a dose capable of treating a subject for a medical disorder as described herein. Effective doses can vary depending upon, for example, the therapeutic compound used, the medical disorder being treated, and the size and type of the recipient animal. Effective doses to treat a subject include doses administered over time that are capable of regulating the activity, including growth, of cells involved in a medical disorder. For example, a first dose of a naked plasmid DNA compound of the present invention can comprise an amount that causes a tumor to decrease in size by about 10% over 7 days when administered to a subject having a tumor. A second dose can comprise at least the same the same therapeutic compound than the first dose.

Another aspect of the present invention includes a method for prescribing treatment for subjects having a medical disorder as described herein. A preferred method for prescribing treatment comprises: (a) measuring the MEKK protein activity in a cell involved in the medical disorder to determine if the cell is susceptible to treatment using a method of the present invention; and (b) prescribing treatment comprising regulating the activity of a MEKK-dependent pathway relative to the activity of a Raf-dependent pathway in the cell to induce the apoptosis of the cell. The step of measuring MEKK protein activity can comprise: (1) removing a sample of cells from a subject; (2) stimulating the cells with a TNFα; and (3) detecting the state of phosphorylation of MKK3, MKK4 or JNKK protein using an immunoassay using antibodies specific for phosphothreonine and/or phosphoserine.

The present invention also includes antibodies capable of selectively binding to a MEKK protein of the present invention Such an antibody is herein referred to as an anti-MEKK antibody. Polyclonal populations of anti-MEKK antibodies can be contained in a MEKK antiserum. MEKK antiserum can refer to affinity purified polyclonal antibodies, ammonium sulfate cut antiserum or whole antiserum. As used herein, the term “selectively binds to” refers to the ability of such an antibody to preferentially bind to MEKK proteins. Binding can be measured using a variety of methods known to those skilled in the art including immunoblot assays, immunoprecipitation assays, enzyme immunoassays (e.g., ELISA), radioimmunoassays, immunofluorescent antibody assays and immunoelectron microscopy; see, for example, Sambrook et al., Molecular Cloning: A Laboratory Manual, Cold Spring Harbor Labs Press, 1989.

Antibodies of the present invention can be either polyclonal or monoclonal antibodies and can be prepared using techniques standard in the art. Antibodies of the present invention include functional equivalents such as antibody fragments and genetically-engineered antibodies, including single chain antibodies, that are capable of selectively binding to at least one of the epitopes of the protein used to obtain the antibodies. Preferably, antibodies are raised in response to proteins that are encoded, at least in part, by a MEKK nucleic acid molecule. More preferably antibodies are raised in response to at least a portion of a MEKK protein, and even more preferably antibodies are raised in response to either the amino terminus or the carboxyl terminus of a MEKK protein. Preferably, an antibody of the present invention has a single site binding affinity of from about 10³M⁻¹ to about 10¹²M⁻¹ for a MEKK protein of the present invention.

A preferred method to produce antibodies of the present invention includes administering to an animal an effective amount of a MEKK protein to produce the antibody and recovering the antibodies. Antibodies of the present invention have a variety of potential uses that are within the scope of the present invention. For example, such antibodies can be used to identify unique MEKK proteins and recover MEKK proteins.

Another aspect of the present invention comprises a therapeutic compound capable of regulating the activity of a MEKK-dependent pathway in a cell identified by a process, comprising: (a) contacting a cell with a putative regulatory molecule; and (b) determining the ability of the putative regulatory compound to regulate the activity of a MEKK-dependent pathway in the cell by measuring the activation of at least one member of said MEKK-dependent pathway. Preferred methods to measure the activation of a member of a MEKK-dependent pathway include measuring the transcription regulation activity of c-Myc protein, measuring the phosphorylation of a protein selected from the group consisting of MEKK, JNKK, JNK, Jun, ATF-2, Myc, and combinations thereof.

Mitogen-activated protein kinase kinase (MEKK1) is a serine/threonine protein kinase that functions parallel to Raf-1 in the regulation of sequential protein kinase pathways that involve both mitogen-activated and stress-activated protein kinases. In this study, we examined the interaction of MEKK1 with 14-3-3 proteins. The T cell 14-3-3 isoform, but not the β and stratifin isoforms, interacted with MEKK1 in the two-hybrid system. GST fusion proteins of the T cell, β, and stratifin 14-3-3 isoforms were prepared to further characterize the domains of MEKK1 and Raf-1 that interact with these proteins. It was demonstrated that the T cell and β 14-3-3 isoform, but not stratifin, interact with COS cell-expressed MEKK1. Furthermore, the amino-terminal moiety, but not the carboxyl-terminal moiety, of expressed MEKK1 interacts with the GST.14-3-3 although the interaction is best when holoMEKK1 is expressed. In contrast, GST.14-3-3 proteins interact with both the amino- and carboxyl-regions of COS cell-expressed Raf-1 protein. Thus, although MEKK1 and Raf-1 function at a parallel point in the sequential protein kinase pathways, the interaction of 14-3-3 proteins with these kinases is not identical, suggesting a differential regulation between Raf-1 and MEKK1 -stimulated pathways.

The foregoing description of the invention has been presented for purposes of illustration and description. Further, the description is not intended to limit the invention to the form disclosed herein. Consequently, variations and modifications commensurate with the above teachings, and the skill or knowledge in the relevant art are within the scope of the present invention. The preferred embodiment described herein above is further intended to explain the best mode known of practicing the invention and to enable others skilled in the art to utilize the invention in various embodiments and with various modifications required by their particular applications or uses of the invention. It is intended that the appended claims be construed to include alternate embodiments to the extent permitted by the prior art.

This invention is further illustrated by the following examples which should not be construed as limiting. The contents of all references, patents and published patent applications cited throughout this application are hereby incorporated by reference.

### EXAMPLE 1

This example describes the structural characterization of MEKK1 protein.

MEKK1 Nucleotide Sequences

MEKK1.1 and 1.2 nucleotide sequences, and encoded proteins, were cloned by the following method. Unique degenerate inosine oligodeoxynucleotides were designed to correspond to regions of sequence identity between the yeast Ste11 and Byr2 genes. With primers and cDNA templates derived from polyadenylated RNA from NIH 3T3 cells, a polymerase chain reaction (PCR) amplification product of 320 base pairs (bp) was isolated. This 320 bp cDNA was used as a probe to identify a MEKK1. 1 cDNA of 3260 bp from a mouse brain cDNA library using standard methods in the art. The MEKK1.1 nucleotide sequence was determined by dideoxynucleotide sequencing of double-stranded DNA using standard methods in the art and is shown in SEQ ID NO: 1.

Referring to SEQ ID No:X, based on the Kozak consensus sequence for initiation codons, the starting methionine can be predicted to occur at nucleotide 486. With this methionine at the start, the cDNA encodes a protein of 672 amino acids, corresponding to a molecular size of 73 kD. When run on a gel, the protein has an apparent molecular size of 69 kD. There is another in-frame methionine at position 441, which does not follow the Kozak rule, but would yield a protein of 687 amino acid residues (74.6 kD). Referring to the MEKK1.1 protein sequence of SEQ ID No:2, 20% of the NH2-terminal 400 amino acids are serine or threonine and there are only two tyrosines. Several potential sites of phosphorylation by protein kinase C are apparent in the NH₂-terminal region. The kinase catalytic domain is located in the COOH-terminal half of the MEKK1.

A longer MEKK1-encoding cDNA was also isolated, referred to as MEKK1.2, the nucleotide and amino acid sequences of which are shown in SEQ ID NO: 3 and 4, respectively.

Immunoblots Using Anti-MEKK Antibodies

Three polyclonal antisera were prepared using three different antigens. A first polyclonal antiserum was prepared using an antigen comprising a 15 amino acid peptide DRPPSRELLKHPVER (SEQ ID NO: 9) derived from the COOH-terminus of MEKK. NZW rabbits were immunized with the peptide and antisera was recovered using standard methods known in the art. This first polyclonal antiserum is hereinafter referred to as the DRPP antiserum (positions 1-4 of SEQ ID NO: 9).

A second polyclonal antiserum was produced using a DNA clone comprising a MEKK cDNA digested with EcoR1 and PstI, thereby creating a 1270 bp fragment that encodes the amino terminus of MEKK. This fragment was cloned into pRSETC to form the recombinant molecule pMEKK1-369 comprising amino acid residues 1 to 369 of MEKK1. The pMEKK1 1-369 recombinant molecule was expressed in E. coli and protein encoded by the recombinant molecule was recovered and purified using standard methods known in the art. NZW rabbits were immunized with the purified recombinant MEKK1 1-369 protein and antisera was recovered using standard methods known in the art. This second polyclonal antiserum is hereinafter referred to as the MEKK1 1-369 antiserum.

A third polyclonal antiserum was produced using a DNA clone comprising a MEKK cDNA digested with Pst 1 and Kpn 1, thereby creating a 1670 bp fragment that encodes the catalytic domain of MEKK. This fragment was cloned into pRSETC to form the recombinant molecule pMEKK1 370-738 comprising amino acid residues 370 to 738 of MEKK1 (encoded by base pairs 1592-3260). The pMEKK1 370-738 recombinant molecule was expressed in E. coli and protein encoded by the recombinant molecule was recovered and purified using standard methods known in the art. NZW rabbits were immunized with the purified recombinant MEKK1 370-738 protein and antisera was recovered using standard methods known in the art. This second polyclonal antiserum is hereinafter referred to as the MEKK1 370-738 antiserum.

The DRPP antiserum was used to probe Western Blots of soluble cellular protein derived from several rodent cell lines. Soluble cellular protein (100 μg) or recombinant MEKK COOH-terminal fusion protein (30 ng) was loaded onto a 10% Tris Glycine SDS-PAGE gel and the protein transferred to a nylon filter using methods standard in the art. The nylon filter was immunoblotted with affinity purified DRPP antiserum (1:300 dilution). A 78 kD immunoreactive protein was identified in the samples comprising protein from Pheochromocytoma (PC12), Rat 1a, and NIH 3T3 cells. A prominent 50 kD immunoreactive band was also commonly present but varied in intensity from preparation to preparation indicating the band is a proteolytic fragment. Visualization of both the 78 kD and 50 kD immunoreactive bands on immunoblots was inhibited by pre-incubation of the 15 amino acid peptide antigen with the affinity purified DRPP antiserum. The MEKK protein detected by immunoblotting is similar to the molecular size predicted from the open reading frame of the MEKK cDNA.

In a second immunoblot experiment, PC12 cells stimulated or not stimulated with EGF were lysed and resolved on 10% Tris Glycine SDS-PAGE gel as described above. MEKK proteins contained in the cell lysates were identified by immunoblot using affinity purified MEKK₁₁-369 antiserum (1:300) using methods standard in the art. MEKK1 and two higher molecular weight proteins having MEKK activity, MEKK α and MEKK β, were identified using the affinity purified MEKK1 1-369 antiserum. MEKK1, and not MEKK α and MEKK β, were identified using the affinity purified MEKK1 1-369 antiserum.

Using the same procedure described above, two MEKK immunoreactive species of approximately 98 kD and 82 kD present in PC12, Rat1a, NIH3T3, and Swiss3T3 cell lysates were recognized by affinity purified MEKK1-369 antiserum. It should be noted that the 98 kD MEKK protein described herein was originally identified as a 95 kD MEKK protein in the related PCT application (International application no. PCT/US94/04178). Subsequent Tris Glycine SDS-PAGE gel analysis has led to the determination that the modification in molecular weight. Visualization of both of these proteins was inhibited by incubation of the affinity purified MEKK1 1-369 antiserum with purified recombinant MEKK1 1-369 fusion protein antigen. A single 98 kD MEKK protein was present in MEKK immunoprecipitates, but not in immunoprecipitates using preimmune serum. More of the 98 kD MEKK was expressed in PC12 cells relative to fibroblast cell lines. Immunoblotting with antibodies that specifically recognize Raf-1 or Raf-B indicated that neither of these enzymes were present as contaminants of MEKK immunoprecipitates. 98 kD MEKK in MEKK immunoprecipitates did not comigrate with Raf-1 or Raf-B in PC12 cell lysates and no cross-reactivity between MEKK and Raf antibodies was observed.

### EXAMPLE 2

This Example describes the activation of a 98 kD MEKK protein isolated from PC12 cells in response to stimulation of cells containing MEKK1 protein by growth factors.

PC12 cells were deprived of serum by incubation in starvation media (DMEM, 0.1% BSA) for 18-20 hours and MEKK1 was immunoprecipitated from lysates containing equal amounts of protein from untreated controls or cells treated with EGF (30 ng/ml) or NGF (100 ng/ml) for 5 minutes with the above-described anti-MEKK1 antibodies speicific for the NH₄-terminal portion of MEKK1. Immunoprecipitated MEKK1 was resuspended in 8 μl of PAN (10 mM piperazine-N,N′-bis-2-ethanesulfonic acid (Pipes) (pH 7.0), 100 mM NaCl, and aprotinin (20 μg/ml)) and incubated with catalytically inactive MEK-1 (150 ng) and 40 μCi of( γ-³²P)ATP in universal kinase buffer (20 mM piperazine-N,N′-bis-2-ethanesulfonic acid (Pipes) (pH 7.0), 10 mM MnCl₂, and aprotinin (20 μg/ml)) in a final volume of 20 μl for 25 minutes at 30° C. Reactions were stopped by the addition of 2× SDS sample buffer (20 μl). The samples were boiled for 3 minutes and subjected to SDS-PAGE and autoradiography. Raf-B was immunoprecipitated from the same untreated and treated PC 12 cell lysates as above with an antiserum to a COOH-terminal peptide of Raf-B (Santa Cruz Biotechnology, Inc.) and assayed similarly. Raf-1 was immunoprecipitated with an antiserum to the 12 COOH-terminal amino acids of Raf-1 (Santa Cruz Biotechnology, Inc.). Epidermal growth factor (EGF) treatment of serum starved PC12 cells resulted in increased MEKK1 activity.

Results were obtained by measuring the phosphorylation of purified MEK-1 (a kinase inactive form) by immunoprecipitates of MEKK1 in in vitro kinase assays. NGF stimulated a slight increase in MEKK1 activity compared to control immunoprecipitates from untreated cells. Stimulation of MEKK1 activity by NGF and EGF was similar to Raf-B activation by these agents, although Raf-B exhibited a high basal activity. Activation of c-Raf-1 by NGF and EGF was almost negligible in comparison to that of MEKK1 or Raf-B.

A timecourse of MEKK1 stimulation by EGF was established by immunoprecipitating MEKK1 or Raf-B protein from lysates of PC12 cells treated with EGF (30 ng/ml) for 0, 1, 3, 5, 10, or 20 minutes and incubating the protein with catalytically inactive MEK-1 (150 ng) and (γ-³²P)ATP as described above. The timecourse of EGF treatment indicated that MEKK1 activation reached maximal levels following 5 minutes and persisted for at least 30 minutes. Raf-B exhibited a similar timecourse; peak activity occurred within 3-5 minutes following EGF treatment and was persistent for up to 20 minutes.

To further dissociate EGF-stimulated MEKK1 activity from that of Raf-B, Raf-B was immunodepleted from cell lysates prior to MEKK1 immunoprecipitation. Raf-B was pre-cleared from lysates of serum-starved PC 12 cells which had been either treated or not treated with EGF (30 ng/ml) for 5 minutes. Raf-B was pre-cleared two times using antisera to Raf-B or using preimmune IgG antisera as a control. The pre-cleared supernatant was then immunoprecipitated with either MEKK1 or Raf-B antisera and incubated with catalytically inactive MEK-1 and (γ-³²P)ATP as described in detail above. EGF-stimulated and unstimulated PC12 cell lysates were precleared with either IgG or Raf-B antisera and then subjected to immunoprecipitation with MEKK1 antiserum or Raf-B antibodies. The results indicate that pre-clearing with Raf-B resulted in a 60% diminution of Raf-B activity as measured by phosphorimager analysis of Raf-B in vitro kinase assays. EGF-stimulated MEKK activity was unaffected by Raf-B depletion, suggesting that Raf-B is not a component of MEKK immunoprecipitates. At least 40% of the Raf-B activity is resistant to preclearing with Raf-B antibodies. Recombinant wild type MEKK1 over-expressed in COS cells readily autophosphorylates on serine and threonine residues and the amino-terminus of MEKK1 is highly serine and threonine rich. MEKK1 contained in immunoprecipitates of PC12 cells were tested for selective phosphorylation of purified recombinant MEKK1 amino-terminal fusion protein in in vitro kinase assays.

Serum-starved PC12 cells were treated with EGF (30 ng/ml) for 5 minutes and equal amounts of protein from the same cell lysates were immunoprecipitated with either MEKK1, Raf-B, or preimmune antiserum as a control. Immunoprecipitates were incubated with purified recombinant MEKK1 NH₂-terminal fusion protein (400 ng) and (γ-³²P)ATP as described above. The results indicate that MEKK1 immunoprecipitated from lysates of EGF-stimulated and unstimulated PC12 cells robustly phosphorylated the inert 50 kD MEKK1 NH₂-fusion protein, while Raf-B or preimmune immunoprecipitates from EGF-stimulated or unstimulated cells did not use the MEKK1 NH₂-fusion protein as a substrate. Thus, the EGF-stimulated MEKK1 activity contained in MEKK1 immunoprecipites is not due to contaminating Raf kinases.

### EXAMPLE 3

This Example describes MEKK1 activity in FPLC Mono Q ino-exchange column fractions of PC12 cell lysates.

Cell lysates were prepared from EGF-stimulated PC12 cells. Portions (900 μl) of 1 ml column fractions (1 to 525 mM NaCl gradient) were concentrated by precipitation with trichloroacetic acid and loaded on gels as described above. The gels were blotted and then immunoblotted with MEKK1 specific antibody. The 98 kD MEKK1 immunoreactivity eluted in fractions 10 to 12. The peak of B-Raf immunoreactivity eluted in fraction 14, whereas Raf-1 was not detected in the eulates from the column. Portions (30 μl) of each fraction from the PC12 lysates of unstimulated control cells or EGF-treated cells were assayed as described above in buffer containing purified recombinant MEK-1 (150 ng) as a substrate. These results indicate that the peak of MEKK1 activity eluted in fractions 10 to 12 from EGF-stimulated PC 12 cells phosphorylated MEK, whereas little MEK phosphorylation occurred in fractions from unstimulated cells.

### EXAMPLE 4

This Example describes studies demonstrating that the phosphorylation of both MEK-1 and the MEKK1 NH₂-terminal fusion protein were due to the activity of the 98 kD PC12 cell MEKK1.

Cell lysates prepared from EGF-stimulated and unstimulated cells were fractionated by FPLC on a Mono-Q column to partially purify the endogenous MEKK1. Lysates from unstimulated control PC12 cells or cells treated with EGF (30 ng/ml) for 5 minutes were fractionated by FPLC on a Mono Q column using a linear gradient of 0 to 525 mM NaCl. A portion (30l) of each even numbered fraction was mixed with buffer (20 mM piperazine-N,N′-bis-2-ethanesulfonic acid (Pipes) (pH 7.0), 10 mM MnCl₂, aprotinin (20 μg/ml), 50 mM β-glycerophosphate (pH 7.2), 1 mM EGTA, IP-20 (50 μg/ml), 50 mM NaF, and 30 μCi (γ-³²P)ATP) containing purified recombinant MEK-1 (150 ng) as a substrate in a final volume of 40 μl and incubated at 30° C. for 25 minutes. Reactions were stopped by the addition of 2× SDS sample buffer (40 μl), boiled and subjected to SDS-PAGE and autoradiography. The peak of MEKK1 activity eluted in fractions 10-12. Portions (30 μl) of each even numbered fraction from lysates of EGF-treated PC12 cells were mixed with buffer as described above except containing purified recombinant MEKK NH₂-terminal fusion protein (400 ng) as a substrate instead of MEK-1. Purified recombinant kinase inactive MEK-1 or the MEKK1 NH₂-terminal fusion protein were then used as substrates in the presence of (γ-³²P)ATP to determine whether 98 kD MEKK1 directly phosphorylates either substrate. Fractions 10-14 of lysate from PC 12 cells treated with EGF phosphorylated MEK-1 while little MEK-1 phosphorylation occurred in untreated control fractions. The MEKK1 NH₂-terminal fusion protein was also phosphorylated in the same fractions as was MEK-1, although the peak of activity was slightly broader (fractions 8-16).

Immunoblotting of column fractions demonstrated that the 98 kD MEKK1 protein co-eluted with the peak of activity that phosphorylated either exogenously added kinase inactive MEK-1 or the 50 kD MEKK1 NH₂-terminal fusion protein. Portions (900 μl) of even numbered column fractions were concentrated by precipitation with trichloroacetic acid and immunoblotted with MEKK1 antibody. The peak of immunoreactivity eluted in fractions 10-12.

### EXAMPLE 5

This Example describes the activation of MEK by a 98 kD MEKK1. 98 kD MEKK1 was immunoprecipitated using the MEKK₁₋₃₆₉ antiserum described in Example 1 from untreated (−) or EGF-treated (+) PC12 cell lysates. The immunoprecipitates were incubated in the presence (+) or absence (−) of purified recombinant wild-type MEK (150 ng) and in the presence of purified recombinant catalytically inactive MAPK (300 ng) and (γ-³²P)ATP. The results indicate that immunoprecipitated MEKK1 from EGF-stimulated cells phosphorylated and activated MEK, leading to MAPK phosphorylation. No phosphorylation of MAPK occurred in the absence of added recombinant MEK. Immunoblotting demonstrated that there was no contaminating MAPK or contaminating MEK in the MEKK1 immunoprecipitates from the EGF-stimulated PC12 cells. Thus, phosphorylation and activation of MEK is due to EGF stimulation of MEKK1 activity measured in the immunoprecipitates.

### EXAMPLE 6

This Example demonstrates the ability of a PPPSS-trunc and Nco1-trunc of MEKK1 protein to activate MAPK activity compared with full-length MEKK1 protein and a negative control protein.

Amino-terminal deletions of MEKK1 were prepared by truncating the protein at an Nco-1 within the corresponding DNA sequence or by truncation at PPPSS (SEQ ID NO: 10, corresponding to amino acids 211-215 of SEQ ID NO: 2). The ability of the truncated forms of MEKK1 to activate MAPK activity was examined. The results indicated that the truncated MEKK1 molecules were more active than the full-length MEKK1. Indeed, the truncated MEKK1 molecules were at least about 1.5 times more active than full-length MEKK1 protein. Thus, removal of the regulatory domain of MEKK1 deregulates the activity of the catalytic domain resulting in improved enzyme activity.

### EXAMPLE 7

This example describes MEKK1 -induced apoptosis.

Cells were prepared for the apoptosis studies as follows. Swiss 3T3 cells and REF52 cells were transfected with an expression plasmid encoding β-Galactoctosidase (β-Gal) detection of injected cells. One set of β-Gal transfected cells were then microinjected with an expression vector encoding MEKK1 370-738 protein. Another set of β-Gal transfected cells were then microinjected with an expression vector encoding truncated BXB-Raf protein.

A. Beauvericin-induced Apoptosis

A first group of transfected Swiss 3T3 cells and REF52 cells were treated with 50 μM beauvericin for 6 hours at 37° C. Beauvericin is a compound known to induce apoptosis in mammalian cells. A second group of cells were treated with a control buffer lacking beauvericin. The treated cells were then fixed in parafornaldehyde and permeabilized with saponin using protocols standard in the art. The permeabilized cells were then labelled by incubating the cells with a fluorescein-labelled anti-tubulin antibody (1:500; obtained from GIBCO, Gaithersburg, Md.) to detect cytoplasmic shrinkage or 10 μM propidium iodide (obtained from Sigma, St. Louis, Mo.) to stain DNA to detect nuclear condensation. The labelled cells were then viewed by differential fluorescent imaging using a Nikon Diaphot fluorescent microscope. The cells treated with beauvericin demonstrated cytoplasmic shrinkage (monitored by the anti-tubulin antibodies) and nuclear condensation (monitored by the propidium iodide) characteristic of apoptosis.

B. MEKK-induced Apoptosis

Swiss 3T3 cells and REF52 cells microinjected with a β-galatoctosidase expression plasmid, and an MEKK encoding plasmid or a BXB-Raf encoding plasmid, were treated and viewed using the method described above in Section A. An anti-β-Gal antibody (1:500, obtained from GIBCO, Gaithersburg Md.) was used to detect injected cells. Microscopic analysis of REF52 cells indicated that the cells expressing MEKK1 protein underwent cytoplasmic shrinkage and nuclear condensation leading to apoptotic death. In contrast, cells expressing BXB-Raf protein displayed normal morphology and did not undergo apoptosis. Similarly, microscopic analysis of Swiss 3T3 cells indicated that the cells expressing MEKK1 protein underwent cytoplasmic shrinkage and nuclear condensation leading to apoptotic death. In contrast, cells expressing BXB-Raf protein displayed normal morphology and did not undergo apoptosis. Thus, MEKK1 and not Raf protein can induce apoptotic programmed cell death.

### EXAMPLE 8

This example describes MEKK1-induced apoptosis, which is independent of JNK/SAPK activation.

Methods

Microinjection

Swiss 3T3 and REF52 cells were plated on acid-washed glass cover slips in Dulbecco's Modified Eagle's Medium (DMEM) and 10% bovine calf serum (BCS) or newborn calf serum (NCS). Cells were placed in DMEM/0.1% calf serum for overnight incubation prior to microinjection and used for injection at 50-70% confluence. Injections were performed with an Eppendorf automated microinjection system with needles pulled from glass capillaries on a vertical pipette puller (Kopf, Tujunga, Calif.). Cells were injected with pCMVβ-gal in the presence or absence of pCMV5MEKK_{COOH} or pCMV5BxBRaf at 20-100 ng/μl for each expression plasmid in 100 mM KC1, 5 mM NaPO₄, pH 7.3. Following injection cells were placed in 1% NCS for 12-18 hr (Swiss 3T3) or 42 hr (REF52) prior to fixation with paraformaldehyde and staining. Similar results were obtained when cells were placed in 10% NCS after microinjection. Propidium iodide (5 pg/ml) was used to stain DNA. X-Gal reactions were performed for six hr.

Swiss 3T3 cells were microinjected with 100 ng/μl pCMVβ-gal and 20 ng/μl pCMV5MEKK_{COOH}. To label free DNA ends fixed and rehydrated cells were incubated with terminal deoxytransferase (TDT) and 10 nM biotin-dUTP following the manufacturer's instructions (Boehringer-Mannheim). Cells were stained with FITC-streptavidin to label DNA fragments. β-gal was detected using rabbit anti-β-gal antibody (Cappel Labs) and a rhodamine-labeled goat anti-rabbit antibody (Cappel Labs).

Transactivation Analysis

Swiss 3T3 cells were transfected using calcium phosphate or lipofectamine with the reporter plasmid Gal4-TK-luciferase, which contains four Gal4 binding sites (Sadowski, I., et al. (1988). Nature 335, 563-564). adjacent to a minimal thymidine kinase (TK) promoter that controls expression of luciferase, in the presence or absence of activator plasmids encoding Gal4₍₁₋₁₄₇₎/Myc₍₇₋₁₀₁₎ (Gupta et al. (1993) Proc. Natl. Acad. Sci. USA 90:3216-3220), Gal4₍₁₋₁₄₇₎/Elk-I₍₈₃₋₄₂₈₎ (Marais, et al. (1993) Cell 73:381-393)or Gal4₍₁₋₁₄₇₎/c-Jun₍₁₋₂₃₃₎ Hibi et al. (1993) Genes & Development 7:2135-2148). Transfections included pCMV5 without a cDNA insert (basal control), pCMV5MEKKCOOH and in some experiments pCMV5BxBRaf. Cells were incubated for 24-48 hr after transfection, lysed and assayed for luciferase activity. Values were normalized to equivalent μg protein for all experiments.

Protein Kinase Assays

JNK/SAPK:

Activity was measured using GST (glutathione S-transferase)-c-Jun (1-79) BOUND to glutathione-Sepharose-4B (Hibi et al. supra). Cells expressing MEKK_{COOH} or control cells were lysed in 0.5% Nonidet P40 (NP40), 20 mM Tris-HCl, pH 7.6, 0.25 NaCl, 3 mM EDTA, 3 mM EGTA, 1 mM dithiothreitol, 1 mM phenylmethylsulfonyl fluoride, 2 mM sodium vanadate, 20 μg/ml aprotinin and 5 μg/ml leupeptin. Lysates were centirfuged at 15,000×g for 10 min to remove nuclei and supernatants (25 μg protein) mixed with 10 μl of GST-c-JUN₍₁₋₇₉₎-Sepharose (3-5 μg of GST-c-Jun₍₁₋₇₉₎). The mixture was rotated at 4° C. for 1 hr, washed 2× in lysis buffer and 1× in kinase buffer (20 mM Hepes, pH 7.5, 10 mM MgCl₂, 20 mM β-glycerophosphate, 10 mM p-nitrophenyl phosphate, 1 mM dithiothreitol, 50 μM sodium vanadate). Beads were suspended in 40 μl of kinase buffer with 10 μCi Of [γ³²P] ATP and incubated at 30° C. for 20 min. Samples were boiled in Laemmli buffer and phosphorylated proteins resolved on SDS/10% polyacrylamide gels. To verify the selectivity of the JNK/SAPK assay cell lysates were fractionated by Mono Q ion exchange chromatography and each fraction assayed as described above. Fractions were also immunoblotted with a rabbit antisera recognizing JNK/SAPK. Only fractions containing immunoreactive JNK/SAPK phosphorylated the GST-c-Jun₍₁₋₇₉₎ protein.

p42/44 ERK MAPK:

ERK activity was assayed after fractionation of cell lysates on DEAE-Sephacel (Heasley, L. E. et al. (1994) Am J. Physiol. 267:F366-F373). Alternatively, ERK activity was assayed following Mono Q ion exchange chromatography as previously described and characterized (Heasley, et al. (1992) Mol. Biol. Cell. 3:545-553). The EGF receptor 662-681 peptide was used as a selective substrate for measuring ERK activity (Russell, M. et al. (1995) Biochemistry. 34:6611-6615.

p38/Hog-1:

Cells were lysed in 1% Triton X-100, 0.5% NP40, 20 mM Tris-HCl, pH 7.5, 150 mM NaCl, 20 mM NaF, 0.2 mM sodium vanadate, 1 mM EDTA, 1 mM EGTA, 5 mM phenylmethylsulfonyl fluoride. Nuclei were removed by centrifugation at 15,000×g for 5 min. Supernatants (200 μg protein) were used for immunoprecipitation of p3 8/Hog-1 using rabbit antiserum raised against the COOH-terminal peptide sequence of p38/Hog-1 (CFVPPPLDQEEMES) (Han, J. et al. (1992) Mol. Endocrinol. 6:2079-2089) and protein A Sepharose. Immunoprecipitates were washed 1× in lysis buffer, 1× in assay buffer (25 mM Hepes, pH 7.4, 25 mM β-glycerophosphate, 25 mM NaCl₂, 2 mM dithiothreitol, 0.1 mM sodium vanadate), resuspended in kinase assay buffer with 20-50 ng of a recombinant NH₂₋ terminal fragment of ATF-2 as substrate and 20 μCi [γ32P] ATP (Abdel-Hafig, et al. (1992) Mol. Endocrinol 6:2079-2089). For verification of the immunoprecipitation assay lysates were fractionated by Mono Q ion exchange chromatography and each fraction assayed for ATF-2 kinase activity and immunoblotted with anti-p38 antibody. The results demonstrated that p38/Hog-1 containing fractions selectively phosphorylated the recombinant ATF-2 protein.

Competitive Inhibitory Mutant JNK/SAPK and JNKK/SEK-1:

The competitive inhibitory JNK/SAPK mutant referred to JNK/SAPK(APF) had the amino acids threonine 183 and tyrosine 185 mutated to alanine and phenylalanine, respectively (Lin et al. (1995) Science 268:286-290). These are the sites phosphorylated by JNKK/SEK-1 and required for activation of the JNK/SAPK kinase activity (Lin et al. supra; Sanchez, I. (1994) Nature 372:794-800). Competitive inhibitory JNKK/SEK-1 was made by mutation of the active site lysine at residue 116 mutated to an arginine (K116R) rendering the protein kinase inactive (Lin et al. supra).

A. Expression of activated MEKK1 Induces Cell Death

Attempts to isolate stable transfectants expressing MEKK_{COOH} in several fibroblast lines failed despite repeated attempts. The fir dings suggested that expression of activated MEKK1 inhibited clonal expansion of transfected cells. For this reason, we characterized the functional consequence of expressing activated MEKK1 in Swiss 3T3 and REF52 cells using nuclear microinjection of an expression plasmid encoding an activated form of MEKK1. Cells were microinjected with an expression plasmid encoding β-galactosidase (β-gal) in the presence or the absence of the expression plasmid encoding MEKK_{COOH}, a truncated activated form of MEKK1 (Yan, M. et al. (1994) Nature 372:798-800; Lange-Carter, C. A., et al. (1993) Science 260:315-319). When Swiss 3T3 cells microinjected with expression plasmids for β-gal alone (control) or β-gal plus MEKK_{COOH} it was readily apparent that expression of the activated MEKK1 induced a strong morphological change of the cells. In contrast, cells microinjected with the β-gal plasmid alone were similar in morphology to uninjected cells. Injected cells became highly condensed with a very dark staining of the cytoplasm that has dramatically shrunken relative to the flattened morphology of the cells injected with β-gal alone. The results indicated MEKKCooH expression resulted in death of the cells.

For further analysis and comparison cells were microinjected with BxBRaf, a truncated activated form of Raf-1 (Rapp, U. R. (1991) Oncogene 6:495-500) that selectively activates the ERK pathway (Kyriakis, J. M. et al. (1992) Nature 358:417-421). In microinjected cells, expression of β-gal, MEKKCOOH or BxBRaf was demonstrated by indirect immunofluorescence using specific antibodies recognizing each protein. Swiss 3T3 cells and REF 52 cells microinjected with the indicated expression plasmid were fixed and stained only eight hours postinjection to demonstrate that each protein was being expressed in the cytoplasm of the cells. It was apparent with the REF 52 cells expressing MEKK began to undergo a morphological changes relative to β-gal expressing cells.

TABLE 2
Quantitation of MEKK_{COOH}-induced cell death.

| DNA Injected | Cells Injected | Condensed Cells |
|---|---|---|
| β-gal | 336 | 4 (1%) |
| β-gal + | 175 | 5 (3%) |
| BxBRaf | | |
| β-gal + | 200 | 167 (84%) |
| MEKK_{COOH} | | |
| β-gal + | 50 | 0 (0%) |
| Kin˜MEKK_{COOH} | | |

Swiss 3T3 cells were injected with solutions containing 100 ng/μl CMV-βgal in the presence or absence of 100 ng/μl of pCMV5-BxEBRaf, pCMVS-MEKKCooH or pCMV5-Kin˜MEKK_{COOH} (kinase inactive mutant; 13). Seventeen hours after injection cells were fixed and stained for β-galactosidase activity with X-Gal. Injected cells attached to the coverslip were scored as positive for cell death when they were highly condensed, small round cells.

The results of this experiment demonstrated that expression of MEKK_{COOH} resulted in significant cell death characterized by the dramatic morphological condensation. In contrast, BxBRaf expression did not affect cell viability relative to control cells expressing only β-gal. Approximately 84% of all MEKK_{COOH} injected cells had a highly condensed cellular morphology seventeen hours after injection. This count actually underestimates the number of condensed cells because Swiss 3T3 cells in advanced stages of the cell death response were often nonadherent to coverslips. Some of the nonadherent highly condensed cells could be found to be released from the coverslip into the culture medium, but were not scored in the quantitation. In contrast, fewer than 3% of BxBRaf and 1% of control β-gal injected cells had an altered morphology even after 48-72 hours post-injection.

These data also show that cell death resulting from MEKK_{COOH} expression required the kinase activity of the enzyme; the kinase inactive mutant of MEKK_{COOH} was without effect. The apoptotic-like cell death was also dependent on the MEKK_{COOH} concentration as measured by serial dilution (0-100 ng/μl) of the expression plasmid used for microinjection. Maintenance of the MEKK_{COOH} expressing cells in 10% serum slightly prolonged the time required for induction of cytoplasmic shrinkage, nuclear condensation and cell death suggesting that growth factors and cytokines had some influence on the onset of the response induced by MEKK_{COOH} but high serum could not prevent MEKKCOOH induced cell death. Greater than 80% of MEKK_{COOH} expressing cells had a cytoplasmic and nuclear morphology characteristic of apoptosis 18 hrs post-injection.

More dramatic morphological changes in Swiss 3T3 cells also resulted from expression of MEKK_{COOH}. Cytoplasmic shrinkage is evident from the β-gal staining and nuclear condensation is evident in MEKK1 expressing cells stained with propidium iodide. In contrast, cells expressing BxBRaf do not demonstrate any detectable morphological difference from control cells expressing only β-gal. Similar dramatic cytoplasmic shrinkage and nuclear condensation was observed with MEKK_{COOH} expression in REF52 cells, where BxBRaf again had no effect on cytoplasmic and nuclear integrity. To assess if DNA fragmentation was induced by MEKK_{COOH} expression, terminal deoxytransferase (TDT) was used to covalently transfer biotin-dUTP to the ends of DNA breaks in situ. Streptavidin-FITC was then used for detection of dUTP incorporated into cellular DNA. Even though Swiss 3T3 cells do not undergo significant DNA degradation and laddering at the nucleosomal level they do generate larger DNA fragments when stimulated to undergo apoptosis (Obeid, L. M. et al. (1993). Science 259:1769-1771). The condensed nuclei of MEKK_{COOH} injected cells were highly fluorescent indicating significant DNA fragmentation. It is also apparent that the cytoplasm has become highly condensed and the condensed chromatin is distinct from the cytoplasm. Microinjected cells not yet undergoing cytoplasmic and nuclear condensation in response to MEKK_{COOH} did not incorporate dUTP into their DNA. Thus, expression of MEKK_{COOH} induced all the hallmarks of apoptosis including cytoplasmic shrinkage, nuclear condensation and DNA fragmentation.

Expression of BxBRaf did not induce a response measured by any of the criteria mentioned above. BxBRaf expressing cells displayed a normal flattened morphology similar to β-gal expressing cells or to uninjected cells. Transient BxBRaf expression in Swiss 3T3 cells stimulated ERK activity and the transactivation function of the Gal4/Elk-1 chimeric transcription factor, whose activation is dependent on phoshorylation by Erk members of the MAPK family (Marais, R., Cell 73:381-393; Gille, et al. (1995) EMBO J. 14:951-962; Price, M. A., et al. (1995) EMBO J. 14:2589-2601). Cumulatively, the results indicate that activation of the Raf/ERK pathway does not induce the cytoplasmic and nuclear changes observed with MEKK.

B. Induction of Activated MEKK Sensitizes Swiss 3T3 Cells to UV-induced Apoptosis

Because stable expression of MEKKCOOH appeared to inhibit clonal expansion of Swiss 3T3 cells under G418 drug selection, clones were isolated having inducible expression of the kinase. The Lac Switch expression system (Stratagene) was used to control the expression of MEKK_{COOH}. Several independent clones were isolated and their properties analyzed in the presence or absence of IPTG-induced expression of MEKK_{COOH}. The parental LacR+ clone expressing only the Lac repressor was used as the control. Clones expressing inducible MEKK_{COOH}, as determined using an antibody recognizing the extreme COOH-terminus of MEKK, showed a small increase in the number of cells having a condensed cytoplasmic and nuclear morphology relative to control cells even in the absence of IPTG-induced MEKK_{COOH}. This is probably due to a basal level of MEKK_{COOH} expression in uninduced cells. The addition of IPTG to the culture media induced the expression of MEKK_{COOH} and resulted in an increase in cells having the condensed morphology relative to the control IPTG-treated LacR+ clone. However, MEKK_{COOH} expressing cells did not growth arrest and only a fraction of the cells assumed a condensed morphology as dramatic as what was observed with microinjection of the MEKK_{COOH} expression plasmid. This maybe related to selection of cells during the cloning procedure that adapted to a low, constitutive level of MEKK_{COOH} expression. Interestingly, no clones were isolated from a total of one hundred fifty that were analyzed that had a significant constitutive MEKK_{COOH} expression measured by immunoblotting. In addition, the level of MEKK_{COOH} expression following IPTG induction is certainly less than that achieved with nuclear microinjection.

It was found that IPTG-induced MEKK_{COOH} expression stimulated signal transduction pathways that made the cells significantly more sensitive to stresses that induce cell death. For example, cells expressing MEKK_{COOH} were highly sensitive to ultraviolet irradiation. Two hours after exposure to ultraviolet irradiation greater than 30% of the MEKK_{COOH} expressing cells became morphologically highly condensed and appeared apoptotic. In contrast, the population of uninduced cells showed no increase in condensed apoptotic-like cells at this time point. Thus, overnight induction of MEKK_{COOH} expression modestly increased the basal index of morphologically condensed cells and primed the cells for apoptosis in response to UV irradiation. The results indicate that MEKK-regulated signal transduction pathways enhance apoptotic responses to external stimuli.

C. Expression of MEKK_{COOH} Stimulates JNK/SAPK and the Transactivation of c-Myc and Elk-1

The ability of MEKK_{COOH} but not BxBRaf expression to induce cell death indicates that each kinase regulates different sequential protein kinase pathways. Cells were incubated for 17 hours in the absence or presence of IPTG and assayed for JNK/SAPK activity. The induction of MEKK_{COOH} expression in Swiss 3T3 cells, as predicted, stimulated JNK/SAPK activity but did not activate either ERK or p38/Hog1 activity. The results indicate that induction of MEKKCOOH results in the activation of JNK/SAPK which phosphorylates GST-c-Jun. Because known substrates for JNK/SAPK are transcription factors, we assayed MEKKCooH inducible clones for transactivation of specific gene transcription. Chimeric transcription factors having the Gal4 DNA binding domain and the transactivation domain of c-Myc, Elk-1 or c-Jun were used for assay of MEKK_{COOH} signaling using a Gal4 promoter-luciferase reporter gene (Hibi et al. supra; Sadowski, I et al. (1988) Nature 335:563-564; Gupta et al. supra; Marais et al. supra.). Surprisingly, IPTG-induced stable expression of MEKK_{COOH} markedly activated the transactivation function of c-Myc and Elk-1 but had little effect on Gal4/Jun activity. This result was unexpected since MEKK_{COOH} transient expression stimulated Gal4/Jun activity, indicating that transient expression of MEKK_{COOH} was capable of transactivating c-Jun function in Swiss 3T3 cells. In addition, the JNK/SAPK activity stimulated by IPTG-induction of MEKK_{COOH} correlated with the characterized JNK/SAPK enzyme by fractionation on Mono Q FPLC. Thus, MEKK_{COOH} expression in stable clones achieved with IPTG-induction selectively regulated Gal4/Myc and Gal4/Elk-1 but not Gal4/Jun even though JNK/SAPK was activated.

The failure of IPTG-induced MEKK_{COOH} expression to activate Gal4/Jun may be related to the multiple c-Jun NH2-terminal phosphorylation sites involved in regulating c-Jun transactivation. Serines 63 and 73 and threonines 91 and 93 are apparent regulatory phosphorylation sites in c-Jun (Kyriakis et al. (1994) Nature 369:156-160; Derijard, B et al. (1994) Cell 76:1025-1037; Pulverer et al. (1991) Nature 353:670-674; Papavassiliou, et al. (1995) EMBO J. 14:2014-2019). Both clusters are proposed to be sites of phosphorylation for ERKs and JNK/SAPKs (Papavassiliou et al. supra). Transient transfection of MEKK_{COOH} activates JNK/SAPK but also activates ERKs (Lange-Carter et al. supra). In contrast IPTG-induction of MEKKCOOH results in the activation of JNK/SAPK but not Erks. The difference in regulation of c-Jun transactivation may be related to the differential phosphorylation of these sites by JNK/SAPK and ERKs.

Expression of activated Raf in Swiss 3T3 cells stimulated Elk-1 transactivation, but not c-Myc or c-Jun transactivation. This result indicates that Elk-1 transactivation alone does not mediate the cell death response in fibroblasts observed with MEKK_{COOH}. Cumulatively, the findings demonstrate that induction of MEKK_{COOH} expression enhances cell death independent of ERK, p38/Hog-1 or c-Jun transactivation in Swiss 3T3 cells and may involve c-Myc transactivation.

D. Inhibitory JNK/SAPK does not Attenuate MEKK stimulated c-Myc Transactivation or Cell Condensation

To determine if JNK/SAPK activation was required for c-Myc transactivation in response to MEKK_{COOH}, Gal4/Myc activation was assayed in the presence or absence of JNK/SAPK(APF). The results are shown in FIG. 19. The JNK/SAPK(APF) was used as a competitive inhibitor of JNK/SAPK for activation by the immediate upstream JNK kinase/SEK-1 enzyme (Kyriakis et al. supra; Sluss, et al (1994). Mol Cell. Biol. 14:8376-8384; Lin et al (1994) Science 268:286-290; Sanchez et al. (1994) Nature 372:794-800). In transient transfection assays, expression of JNK/SAPK(APF) inhibited approximately 65% of the Gal4/Jun activation in response to MEKK_{COOH}. In contrast, expression of JNK/SAPK(APF) had no effect on MEKK_{COOH} activation of Gal4/Myc induction of luciferase activity. Thus, c-Jun transactivation appears to be independent of the MEKK_{COOH} stimulated pathway leading to c-Myc transactivation. Similarly, JNK/SAPK activation can be significantly inhibited with no effect on c-Myc transactivation.

The cell death response to MEKK_{COOH} also appeared to be largely independent of JNK/SAPK. In several experiments, expression of JNK/SAPK(APF) alone had no demonstrative effect on Swiss 3T3 cells. The expressed JNK/SAPK(APF) was localized in both the cytoplasm and nucleus while β-gal expression was restricted to the cytoplasm. Co-expression of JNK/SAPK(APF) with MEKK_{COOH} did not block MEKK_{COOH}-induced cytoplasmic shrinkage and cellular condensation. A 20-fold lower concentration of MEKK_{COOH} still induced the cytoplasmic shrinkage characteristic of apoptosis in microinjected Swiss 3T3 cells. Co-microinjection of a 30-fold greater concentration of JNK/SAPK(APF) plasmid relative to the MEKK_{COOH} plasmid did not affect the MEKK_{COOH}-mediated cell death response. Cells undergoing a dramatic cytoplasmic shrinkage. Because of the low amount of MEKK_{COOH} expression plasmid used, the cell condensation response was slower in onset. The percentage of MEKK_{COOH} microinjected cells committed to cytoplasmic shrinkage and cellular condensation and the timing of this response was the same in the presence or absence of JNK/SAPK(APF). In addition, the competitive inhibitory mutant K116RJNKK/SEK-1, the kinase immediately upstream of JNK/SAPK which phosphorylates and activates JNK/SAPK (Lin et al supra; Sanchez, 1 (1994) Nature 372:794-800) also unable to attenuate MEKK_{COOH} induced cell death. Expression of JNK/SAPK(APF) or K116RJNKK/SEK-1 alone had no measurable effect on the morphology of Swiss 3T3 cells. Thus, MEKK_{COOH} induces cell death via the regulation of signal pathways that appear largely independent of JNK/SAPK regulation and c-Jun transactivation. Finally, BxBRaf neither induced cell death nor activated c-Myc indicating that MEKK_{COOH}-regulated responses were not mediated by the Erk1 and 2 proteins (p42/p44 MAP kinases), consistent with the lack of ERK activation in the inducible MEKK_{COOH} Swiss 3T3 cells.

These results demonstrate, for the first time, a role for MEKK in mediating a cell death response characteristic of apoptosis. Receptors such as the cytotoxic TNFα receptor and Fas must be capable of regulating signal transduction pathways controlling cytoplasmic and nuclear events involved in apoptosis. The enhanced apoptosis to ultraviolet irradiation observed with MEKK_{COOH} expression in Swiss 3T3 cells indicates that MEKK-regulated signal transduction pathways integrate with the apoptotic response system. MEKK_{COOH} expressing cells have a higher basal apoptotic index and are primed to undergo apoptosis in response to a stress stimulation. The short time required to observe the enhance apoptosis (2 hr) suggests that cell cycle traverse, DNA synthesis, or significant transcription/translation is not required for the enhanced cell death in response to ultraviolet irradiation in cells expressing MEKK_{COOH}. This finding is striking and suggests that genetic or pharmacological manipulation of MEKK activity could be used to sensitize cells to irradiation-induced death.

The ability to dissociate c-Jun transactivation from MEKK_{COOH}-stimulated cell death argues that the JNK/SAPK activity achieved in the inducible Swiss 3T3 cell clones is insufficient alone to activate c-Jun transactivation or induce cell death. It is more likely that the JNK/SAPK activity we have measured is involved in stimulating a protective program in response to potentially lethal stimuli as previously proposed (Devary, Y et al. (1992) Cell 71:1081-1091). Protective responses could involve changes in metabolism or alterations in the activity of proteins such as Bcl-2 (Gottschalk, A. R., et al. (1994) Proc. Natl. Acad. Sci. USA 91:7350-7354; Korsmeyer, S. J. (1992) Immunol. Today 13:285-290). This prediction is consistent with the activation of JNK/SAPK mediated by CD40 ligation in B cells which protects against rather than stimulates apoptosis (Sumimoto, S. I., et al. (1994) J. Immunol. 163:2488-2496; Tsubata, T. et al. (1993) Nature 364:645-648).

Recently, it was shown that dominant negative c-Jun could protect neurons from serum deprivation-induced apoptosis (Ham, J. et al. (1995) Neuron 14:927-939). It was proposed that the dominant negative cJun inactivated c-Jun and prevented an attempt by the post mitotic neurons to enter an abortive cell cycle progression that triggered a cell death program. Thus, dominant negative c-Jun was believed to maintain the neurons in stringent growth arrest. At first glance, the protective effect of dominant negative c-Jun seems contradictory to our results that JNK/SAPK and c-Jun transactivation are not involved in MEKK-induced cell death. Our results demonstrate that the dramatic cytoplasmic shrinkage, nuclear condensation and onset of cell death induced by MEKK_{COOH} are largely independent of JNK or c-Jun transactivation. Importantly, MEKK_{COOH}-induced cell death occurs in high serum where growth factor and cytokine stimulation of the cells is normal. We have also determined that expression of MEKK_{COOH} in Swiss 3T3 cells does not significantly inhibit or alter cell cycle progression. Thus, an abnormal cell cycle event that may occur with serum deprivation does not appear to account for MEKK-induced cell death.

Expression of MEKK_{COOH} increased the transactivation of c-Myc and Elk-1 in Swiss 3T3 cells. c-Myc has been shown to be required for apoptosis in lymphocytes (Fanidi, A et al. (1994) Nature 359:554-556; Janicke, R. U. et al (1994) Mol. Cell. Biol. 14, 5661-5670; Shi et al. (1992) Science 257:212-214), to induce apoptosis when overexpressed in growth factor-deprived fibroblasts (Harrington, E. A. et al. (19Q4) EMBO J. 13:3286-3295); Askew, D. W., et al. (1991) Oncogene 6:1915-1922; Evan, G. I. et al. (1992) Cell 69:119-128), and to enhance TNF-mediated apoptosis (Klefstrom, J., et al. (1994) EMBO J. 13:5442-5450). The requirement of c-Myc for apoptosis is not understood mechanistically, but c-Myc is proposed to transcriptionally activate an apoptotic pathway (Harrington, E. A. et al. (1994) EMBO J. 13:3286-3295); Askew et al. supra; Evan et al. supra, Janicke et al. supra; Shi et al. supra). The activation of Elk-1 by MEKK_{COOH} induction in Swiss 3T3 cells correlates best with the stimulation of JNK/SAPK. Recently, it was found that JNK/SAPK in addition to Erks phosphorylated and activated Elk-1 consistent with our findings (Whitmarsh, A. J. et al. (1995) Science 269:403-407). In contrast, we demonstrate that c-Jun is not significantly activated in MEKK_{COOH} expressing cells. These findings are provocative because they indicate that MEKK-stimulated JNK/SAPK activation preferentially regulated Elk-1 and not c-Jun. A second signal in addition to JNK/SAPK may be required for c-Jun transactivation in cells (Papavassiliou, A. G., et al. (1995) EMBO J. 14:2014-2019). There does not seem to be a proposed role for Elk-I in inducing an apoptotic response, but serum deprivation-induced apoptosis of Swiss 3T3 cells results in the increased expression of early cell cycle genes consistent with an increased SRF/SRE activity associated with elevated Elk-I activity (Pandey, S. and Wang, E. (1995) J. Cell. Biochem. 58:135-150). The induction of apoptosis in several cell types does not appear to require transcription, but the use of inducible cell lines and plasmid microinjection experiments do not facilitate testing whether MEKK_{COOH} can induce cell death in the absence of transcription. In cells where transcription is not necessary for the induction of apoptosis it is likely that proteins required for apoptosis are already expressed and may be post translationally regulated by sequential protein kinase pathways involving MEKK. For example, the phosphorylation of nuclear proteins could alter their activity independent of transcription and contribute to a cell death response.

In Jurkat cells, a human T cell line, Fas-induced apoptosis has been proposed to involve a ceramide stimulated, Ras-dependent signaling pathway (Gulbins, E., et al. (1995) Immunity 2:34351). We recently demonstrated that MEKK activity can be stimulated by Ras and that MEKK1 physically binds to Ras in a GTP-dependent manner (Russell, M. et al. (1995) J. Biol. Chem. 270:11757-11760; Winston, B. W., et al. (1995) Proc. Natl. Acad. Sci. USA (1995) 92:1614-1618). The ability of MEKK to regulate an apoptotic-like cell death response suggests it is a candidate component for the ceramide regulated apoptotic pathway.

The importance of our observations describing the involvement of MEKK regulated sequential protein kinase pathways in physiologically relevant signaling leading to cell death is supported by several findings. First, MEKK_{COOH} induces or enhances a cell death response in the presence of 10% calf serum, indicating that growth factor deprivation is not a prerequisite for MEKK-induced cell death. This is similar to TNFα, Fas and ceramide-mediated apoptosis which proceeds in high serum. Thus, the involvement of MEKK in cell death responses is not simply to activate a subset of growth factor stimulated signaling events causing an aborted cell cycle-induced apoptosis that would normally be prevented by serum factors. Second, the enhanced cell death to ultraviolet irradiation indicates that expression of MEKK_{COOH} may activate signals that potentiate stresses to the cell. This finding indicates that MEKK-regulated signal transduction pathways integrate with cellular responses involved in mediating apoptosis, that ultraviolet irradiation likely activates additional pathways and that MEKK_{COOH}-mediated signaling synergizes with the ultraviolet response to accelerate apoptosis. Third, MEKK stimulated sequential protein kinase pathways independent of ERK, JNK/SAPK, p38/Hog1 and c-Jun transactivation that can stimulate c-Myc transactivation. These results indicate that MEKK-regulated pathways traverse the cytoplasm to regulate as yet undefined protein kinases that activate cMyc in the nucleus. The regulation of c-Myc activity is a unique function of MEKK signaling and one that we postulate is likely to contribute to the cell death response. Serum deprivation significantly induces JNK/SAPK activation in several cell types including Swiss 3T3 cells. Similarly, TNF α stimulates a JNK/SAPK pathway (Minden et al. (1994) Science 266:1719-1723) and we have recently demonstrated TNFα stimulation of MEKK activity in mouse macrophages (Winston et al. supra). c-Myc overexpression has been shown to enhance TNFα receptor stimulation of apoptosis (White et al. (1992) Mol. Cell. Biol. 12:2570-2580). These findings are consistent with a linkage between TNFα receptor signaling, MEKK and c-Myc. Cumulatively, the findings define MEKK as a potentially important component in the regulation of signal transduction pathways involved in apoptosis.

### EXAMPLE 9

This example illustrates that TNF and expression of MEKK1_{COOH} synergize to induce apoptosis in cells.

Control L929 fibroblasts (4.1 LAC1), fibroblasts expressing MEKK1_{COOH} domain (15.10 LAC1), or fibroblasts expressing the kinase inactive mutant of MEKK1_{COOH} (41.112 LAC1) using the Lac Switch expression system, were treated with TNF in the presence or absence of IPTG and the percentage of apoptotic cells was calculated. Approximately 20% of control L929 cells became apoptotic upon TNF exposure either in the presence and absence of IPTG. In L929 cells expressing the MEKK1_{COOH} domain, exposure to TNF and IPTG increased the percentage of apoptotic cells to 40%, approximately a 2-fold increase. In L929 cells expressing the MEK kinase inactive mutant, exposure to TNF did not increase the level of apoptotic cells above levels seen in controls, in fact the percentage of apoptotic cells was slightly decreased in cells exposed to both TNF and IPTG.

### EXAMPLE 10

This example demonstrates Fibroblast Growth Factor-2 suppression of Tumor Necrosis Factor-α mediated apoptosis requires Ras and the Activation of Mitogen-Activated Protein Kinase.

Tumor necrosis factor a (TNFα) is a multifunctional cytokine secreted primarily by activated monocytes (Tracy, K. J., and Cerami, A. (1993) Annu. Rev. Cell Biol. 9:317-343). It has a wide range of biological activities depending upon cell type, stage of differentiation and transformation state. TNFα acts as a growth factor for fibroblasts (Vilcek, J.,et al.(1986) J. Exp. Med. 163:632-643; Victor, I., et al.(1993) J. Biol. Chem. 268:18994-18999), is cytotoxic towards certain cells and tumors (Larrick, J. W., and Wright, S. C. (1990) FASEB J 4:3215-3216), induces monocyte differentiation of the human HL-60 myeloid leukemia cell line (Trinchieri, G., et al. (1986) J. Exp. Med. 164:1206-1225; Kim, M., et al. (1991) J. Biol. Chem. 266:484-489), represses adipocyte (Torti, F. M., et al. (1985) Science 229:867-869) and myoblast differentiation (Miller, S. C., et al. (1988) Mol. Cell. Biol. 8:2295-2301), and mediates endotoxic shock (Tracey, K. J., et al. (1986) Science 234:470-474). The peiotropic effects of this cytokine make it an important mediator in processes as diverse as proliferation, differentiation and cytotoxicity.

TNFα exerts these responses by binding to two cell surface receptore, the 55 kD TNFR (p55 TNFR) and the 75 kD TNFR (p75 TNFR) (Loetscher, H., et al. (1990) Cell 61:351-359; Schall, T. J., et al.(1990) Cell 61:361-370; Smith, C. A., et al. (1990) Science 248:1019-1023; Heller, R. A., et al. (1990) Proc. Natl. Acad. Sci. (USA) 87:6151-6155). The receptors are single transmembrane spanning glycoproteins present on almost all cells analyzed (Kull, Jr., et al. (1985) Proc. Natl. Acad. Sci (USA) 82:5756-5760; Lewis, M., et al. (1991) Proc. Natl. Acad. Sci. (USA) 88:2830-2834). The extracellular domain of the p55 TNFR is homologous to the extracellular domains of the low affinity nerve growth factor receptor, the Fas/APO1 receptor, CD40, OX40, and CD27. The p55 TNFR and Fas share a 65 residue homology region in the cytplasmic domains (Tartaglia, L. A., and Goeddel, D. V. (1992) Immunol. Today 13:151-153; Smith, C. A., et al.(1994) Cell 76:959-962) which deletion studies have implicated in the TNFα signaling cascade leading to apoptosis (Itoh, N., and Nagata, S. (1993) J. Biol. Chem. 268:10932-10937; Tartaglia, L. A., et al. (1993) Cell 74:845-853). Most of the known TNFα responses occur by activation of the p55 TNFR. However, thymocyte proliferation is associated with p75 TNFR and eytotoxicity may be a function of p75 TNFR acting alone or in concert with the p55 TNFR (Heller, R. A., et al. (1992) Cell 70:47-56).

Apoptosis involves the activation of a specific suicide program within a cell. It occurs when a cell initiates a series of biochemical and morphological events which result in nuclear disintegration and eventual fragmentation of the dying cell into a cluster of membrane-bound apoptotic bodies (Kerr, J., Wyllie, A., and Currie, A. (1972) Br. J Cancer 26:239-257). Apoptosis is responsible for such diverse activities as the elimination of cells during normal embryological development and determination of the immune receptor repertoire (Raff, M. C. (1992) Nature 356:297-300; Krammer, P. H., et al. (1994) Curr. Opin. in Immunol. 6:279-289; Green, D. R., and Scott, D. W. (1994) Curr. Opin. in Immunol. 6:476-487) ). Apoptosis can be triggered in multiple ways, but it is not yet known whether different inducers of apoptosis have a common pathway or whether there are multiple pathwyas with perhaps some common components.

In many peptide-hormone receptor systems signal transduction to the nucleus involves the sequential activation of protein kinases. The extracellular response kinase (ERK) group of mitogen-activated protein kinases (p42 and p44 MAPK) are activated by growth factors via a Ras/Raf dependent signal transduction pathway (Davis, R. J. (1993) J. Biol. Chem. 268:14553-14556; Cano, E. and Mahadevan, L. (1995) Trends Biochem. Sci. 20:117-122). In contrast, the JNK/SAPK (Jun kinase/stress-activated protein kinase) members of MAPKs are activated by proinflammatory cytokines and environmental stresses (Devary, et. al. (1992) Cell 71:1081-1091; Hibi, M., et al.(1993) Genes & Development 7:2135-2148; Sluss, H., et al. (1994) Mol. Cell. Biol. 14:8376-8384; Kyriakas, J. M., et al. (1994) Nature 369:156-160; Minden, A., et al. (1994) Mol Cell. Biol. 14:6683-6688).

TNFα has been shown to initiate apoptotic cell death and DNA fragmentation in several mammalian cell lines, including the murine fibrosarcoma cell line L929 (Kyprianou,

N., et al. (1991) J. Natl. Cancer Inst. 83:346-350; Feshel, K., et al.(1991) Am. J Pathol. 139:251-254). RNFa also has been shown to activate p42/p44 MAPK in this cell line (Van Lint, J., et al. (1992) J. Biol. Chem. 267:25916-25921). Recently JNKs were shown to be activated by TNFa (Westwick, J., et al. (1994) J. Biol. Chem. 269:26396-6401) and activation of the JNK pathway correlated with enhanced apoptosis of PC12 cells in response to trophic factor deprivation (Xia, Z., et al. (1995) Science 270:1326-1331). We have characterized the regulation of MAPKs and JNKs in L929 cells challenged with TNFa and basic fibroblast growth factor (bFGF). We show that TNFα preferentially activates JNK in L929 cells; but that JNK activation is not sufficient to induce apoptosis, since bFGF mediates a protective effect against TNFα mediated apoptosis without affecting JNK activation. Furthermore, our data indicate that p42/p44 MAPK activation is required for bFGF supression of TNFa mediated apoptosis.

Materials and Methods

Cell lines and culture. L929 cells (ATCC CCL1 were maintained in Dulbecco's modified Eagle's medium with 5% newborn claf serum and 5% bovine calf serum (BCS) supplemented with 100 ug/ml streptomycin and 100U/ml penicillin. The cells were grown in 10 cm dishes at 37° C. in 7.5% CO2. Cells were made quiescent where indicated by incubation in Dulbecco's modified Eagle's medium and 0.1% bovine serum albumin for 24 h. Recombinant murine TNFα and recombinant humanbFGF (147aa) were from R&D Systems, Minneapolis, Minn. Cells were pretreated where indicated with the MEK-1 inhibitor PD#098059 (Parke-Davis Pharmaceutical Corp. Ann Arbor, Mich.) for 1 h at 37° C. Cells were stimulated by incubation with the indicated cytokine or growth factor for various times at 37° C. Cells were stimulated by incubation wit the indicated cytokine or growth factor for various times at 37° C. Stimulation was stopped by rinsing the plates twice with ice cold phosphate buffered saline (PBS) and lysing the cells in the appropriate lysis buffer. Cells were scraped from the plates and nuclei were pelleted for 10 min at 14,000 RPM in a microcentrifuge.

JNK assay. JNK activity was measured using a solid state kinase assay in which glutathione S-transferase-c-Jun₍₁₋₇₉₎ (GST-JUN) cound to glutathione-Sepharose 4B beads was used to affinity pruify JNK and then JNK activity was measured in an in vitro kinase assay using the sepharose bound GST-Jun as a substrate (Hibi, M., et al. (1993) Genes & Development 7:2135-2148). Stimulated or unstimulated cells were lysed in 0.5% Nonidet P-40, 20 mM HEPES pH 7.2, 100 mM NaCl, 2 mM dithiothreitol, lmM EDTA, 1.0 mM phenylmethylsulfonylfluoride, 1 μg/ml aprotinin and the nuclei pelleted. Lysates were normalized for protein content. JNK was affinity purified from 50-100 μg of cell lysate by the addition of 10 ul of GST-Jun sepharose slurry (2 μg GST-Jun). Binding to GST-Jun efficiently isolates the two major forms of JNK (p45and p55) and under the conditions used JNK isolation was linear for 10-250 μg of cell lysate. The lysates were rotated at 4° C. for 1-3 h. Beads were washed twice in lysis buffer and then twice in PAN (10 mM PIPES, pH 7.0, 100 mM NaCl, 21 μg/ml aprotinin). Kinase reactions were carried out at 30° C. for 15 min in 20 mM Hepes pH 7.2, 20 mM β-glycerophosphate, 10 mMp-nitrophenyl phosphate, 10 mM MgCl₂, 1 mM dithiothreitol, 50 μm sodium vanadate, 10 μCi γ³²P-ATP 4300 Ci/mmole. The kinase reaction was linear from 0-30 min.

MAPK Assay MAPK activity was measured exactly as described previously (Gardner, A. M., et al. (1994) Meth. Enzymol. 238:258-270) with the exception that MonoQ FPLC fractionation was replaced by step elution from a DEAE-Sephacel column using 0.5 M NaCl in loading buffer. The eluate was assayed in triplicate using the epidermal growth factor receptor 662-681 peptide (EGFR₆₆₂₋₆₈₁) as a selective substrate for MAPK activity (Heasley, L. E., et al. (1994) American Journal of Physiology (Renal Fluid Electrolyte Physiol. 36) 267:F366-F373).

RafActivation Assay Cells were serum starved and challenged in the presence or absence of the appropriate cytokine or growth factors, as described above. Cells were lysed by scraping in ice cold RIPA buffer (50 mM Tris-HCl, pH 7.2, 150 mM NaCl, 0.1% SDS, 0.5% sodium deoxycholate, 1.0% Triton X-100, 10 mM sodium pyrophosphate, 25 mM β-glycerophosphate, 2 mM sodium vanadate, 2.1 pg/ml aprotinin) and the nuclei were pelletted. The supernatants were normalized for protein content and precleared with protein A Sepharose prior to immunoprecipitation with rabbit antiserum to the C terminus of C-Raf, rabbit anti-serum to A-Raf or rabbit antiserum to B-Raf (Santa Cruz Biotech., Santa Cruz, Calif.) and protein A Sepharose for 2-3 hr at 4° C. The beads were washed twice with ice cold RIPA and twice with PAN. A third of the immunoprecipitate was diluted with SDS sample buffer and used for immunoblot analysis. The remainder was resuspended in kinase buffer (20 mM Pipes pH 7.0, 10 mM MnCl₂, 150 ng kinase-inactive MEK-1, 30 μCi γ³²P-ATP and 20 μg/ml aprotinin) in a final volume of 40 μl for 30 min at 30° C. Wild-type recombinant MEK-1 was autophosphorylated in parallel as a marker. Reactions were terminated by the addition of 12.5 μl 5× SDS sample buffer, boiled, and subjected to SDS-PAGE and autoradiography.

Neutral Red Assay Uptake of the dye neutral red was used as one measure of cell viability following cytokine or growth factor treatment (Finter, N. B. (1969) J. Gen Virol. 5:419-427). 1.5×10⁴-2.5×10⁵ L929 cells/well were plated in 12 well tissue culture dishes in 1.25 ml of media. Cells were treated for 15-20 hr with various concentrations of TNFα and/or bFGF. 2.5 μl of 1% neutral red was added to the wells and incubated for 2 hr at 37° C. PBS. The neutral red was extracted with 1.0 ml of 50% ethanol, 50 mM Na-citrate pH 4.2 and absorbency was measured at 540 mM.

Propidium iodide staining Cells were plated on glass chamber slides (Nunc, Naperville, Ill.) at a concentration of 0.2-0.6×10⁵ cells/ml. Ras expression was induced with 5 mM IPTG in Dulbecco's modified Eagle's medium with 0.1% BCS for 8-12 hr. Cells were exposed to TNFα (5 ng/ml) and/or bFGF (500 pg/ml) in Dulbecco's modified Eagle's medium with 0.1% BCS for 16 hr. The parental LACI expressing cell line (see below) was used as a control. Cells were washed twice in PBS, fixed in acetone:methanol (1:1) −20° C. for 5 min, air dried, washed twice in PBS, stained with 1 μg/ml propidium iodide (PI) in PBS for 20 min, washed in PBS, washed in H₂O and mounted in 25% glycerol/PBS. PI fluorescence was observed using a Nikon inverted microscope equipped with epifluorescence and a 580 mm filter. Images were analyzed using IP lab.

Cell transfections L929 cells were transfected by CaPO₄ (Ausubel, F. (1994) Current Protocols in Molecular Biology Vol. 1, pp. 9.1.1-9.1.4, John Wiley & Sons, Inc., New York) with the vector 3′SS (Stratagene, La Jolla, Calif.) expressing the LACI repressor. Stable clones were selected in 200 μg/ml hygromycin (Calbiochem, La Jolla, Calif.) and screened for LACI expression by indirect immunofluorescence using rabbit anti-sera to LACI (Stratagene, La Jolla, Calif.) and FITC-donkey anti-rabbit. One clone expressing a high level of nuclear LACI was then transfected with hemaglutinin (HA)-tagged inhibitory N17 (Feig, L. A. and Cooper, G. M. (1988) Mol. Cell. Biol. 8:3235-3243) Ras or activated V12 Ras (Tobin, C.,et al.(1982) Nature 300:143-148; Reddy, E. P., et al.(1982) Nature 300:149-152); Taparowsky, E., Suard, Y., Fassano, D., Simiger, K., Goldfarb, M., and Wigler, M. (1982) Nature 300:149-152) cloned into the LACI repressible pOPRSVI vector. Stable clones were selected in 500 μg/ml G418 and screened for inducible expression of HA-Ras by immunoblotting. Incubation in 5 mM isopropyl-1-thio-β-D-galactopyranoside (IPTG) for 8-24 hr was used to induce Ras expression. Several independent, inducible N17 Ras or V12 Ras clones were isolated and two each were chosen for further analysis.

Immunoblotting 100 μg of cell lysate was fractionated by SDS PAGE (12.5% acrylamide) and blotted to nitrocellulose in 10 mM CAPS, pH 11, 20% MeOH using a Transphor apparatus (Hoeffer, San Diego, Calif.) for 1 hr at 1 amp. Blots were blocked in 5% powdered milk in Tris-HCl, pH 7.5 buffered saline. Ras was detected with Y-13259 anti-Ras monoclonal antibody (Fruth, M. E., Davis, L. J., Fleurdelys, B., and Skolnick, E. M. (1982) J. Virol. 43:294-304) followed by enhanced chemiluminescence (Amersham, Chicago, Ill.) using HRP-anti-mouse IgG (BioRad, Richmond, Calif.).

Quantitation of data Phosphorlmager analysis of phosphorylated proteins provided a quantitative measure of kinase activation in arbitrary phosphorimaging units. Statistical analysis was performed using the JMP program and the method of Tukey & Kramer was used to determine statistical differences.

Results

bFGF protects L929from TNFα-mediated apoptosis TNFα activates a cell death program resulting in the apoptosis of L929 cells (Feshel, K., Kolb-Bachofen, V., and Kolb, H. (1991) Am. J Pathol. 139:251-254). Treatment of L929 cells overnight with TNFα resulted in substantial cell death using the neutral red assay as a measure of viable cells (see Methods). The time course of cell death was dependent on the concentration of TNFα. Treatment with 10 ng/ml TNFα resulted in greater than 40% of the L929 cells being apoptotic in 15 hr; 1 ng/ml TNFα required 24-48 hr to induce a similar level of L929 cell death. Serum and growth factor withdrawal induces apoptosis in several cell systems (Oppenheim, R. W. (1991) Annu. Rev. Neurosci. 14:453-501; Kinoshita, T., et al.(1995) EMBO J 14:266-275), indicating that growth factors have a protective effect against apoptosis. Consistent with this observation was our finding that bFGF affected TNFα mediated apoptosis. Incubation of L929 cells with TNFα in the presence of bFGF was effective at blocking TNFα-mediated cell death. The protective effect of bFGF was not simply due to an increased proliferative response of L929 cells, because bFGF in the absence of TNFα did not measurably increase cell number.

Regulation of JNK and MAPK by TNFα and bFGF TNFα has been previously shown to activate p24/p44 MAPK in L929 cells (Van Lint, J., Agostinis, P., Vandevoorde, V., Haegeman, G., Fiers, W., Merlevede, W., and Vandenheede, J. (1992) J. Biol. Chem. 267:25916-25921) but recent studies have indicated that TNFα is a potent activator of the Jun kinase (JNK) members of the MAPK family (Sluss, H., et al. (1994) Mol. Cell. Biol. 14:8376-8384; Kyriakas, J. M., et al. (1994) Nature 369:156-160; Westwick, J., Weitzel, C., Minden, A., Karin, M., and Brenner, D. (1994) J. Biol. Chem. 269:26396-6401). Analysis of the time course and dose response of TNFα on L929 cells demonstrated significant differences in the activation of JNK and p42/p44 MAPK activity. Extracts from TNFα-treated versus control L929 cells were assayed for JNK activity using GST-c-Jun₍₁₋₇₉₎ as substrate. TNFα induced a transient increase in JNK activity that peaked at 10-15 min and returned to two-fold above basal JNK activity 1-2 hr post-stimulation. Maximal JNK activation was achieved at 1 ng/ml TNFα and 0.1 ng/ml TNFα activated JNK greater than four-fold. TNFα stimulation of p42/p44 MAPK activity was slightly more rapid than JNK activation, reaching maximal stimulation in 5-10 min that returned to near basal levels by 30 min. The dose-response curve for p42/p44 MAPK activation is dramatically shifted to higher TNFα concentrations than that for JNK. Greater than 10 ng/ml TNFA was required to stimulate p42/p44 MAPK 2-3 fold; at 1 ng/ml TNFα the MAPK activity was stimulated only 20% above basal, a concentration of TNFα that gave maximal JNK activation. Thus, TNFα-preferentially regulates the JNK pathway relative to p42/p44 MAPK in L929 cells. These findings indicate that the localized concentration of cytokines such as TNFA will determine the selectivity and magnitude of cellular JNK and p42/p44 MAPK responses.

In contrast to proinflammatory cytokines such as TNFα, growth factor receptor tyrosine kinases are generally mitogenic in fibroblasts and stimulate the p42/p44 MAPK pathway. The bFGF receptor possesses intrinsic tyrosine kinase activity and is present on L929 cells. bFGF stimulated a robust activation of MAPK in L929 cells. Concentrations of 0.25-0.5 ng/ml of bFGF gave maximal stimulation of MAPK activity. Fractionation of stimulated cell lysates by MonoQ fast pressure liquid chromatography indicated that both p42 and p44 MAPK were activated by bFGF. Activation of the MAPK pathway by tyrosine kinases involves Ras and the Raf serine-threonine protein kinases. Immunoblotting demonstrated that B-Raf and C-Raf are expressed in L929 cells. Treatment of L929 cells with bFGF resulted in the activation of both B-Raf and C-Raf as measured by their ability to phosphorylate a recombinant kinase-inactive MEK-1 protein (Gardner, A. M., Lange-Carter, C. A., Vaillancourt, R. R., and Johnson, G. L. (1994) Meth. Enzymol. 238:258-270). MEK-1 is the protein kinase phosphorylated and activated by Raf, which in turn phosphorylates MAPK on both a tyrosine and threonine resulting in MAPK activation (Crews, C. M., Allesandrini, A., and Erikson, R. L. (1992) Science 258:478-480; Crews, C. M., and Erikson, R. L. (1992) Proc. Natl. Acad. Sci. (USA) 89:8205-8209; Nakielny, S., et al. (1992) EMBO J. 11:2123-2129; Seger, R., et al.(1992) J. Biol. Chem. 267:14373-14381). In contrast, TNFα does not significantly activate either isoform of Raf in L929 cells.

bFGF and TNFα independently regulate cytoplasmic protein kinase cascades 1 ng/ml TNFα had only modest stimulatory effects on MAPK activity and 2.5 ng/ml bFGF had little or no effect on JNK activity. These concentrations of bFGF and TNFα give maximal activation of MAPK and JNK, respectively. Co-stimulation of L929 cells with bFGF, at concentrations that show partial protection against TNFα-mediated killing, did not alter the magnitude of JNK activation in response to TNFα. Similarly, co-stimulation of L929 cells with TNFα, at concentrations capable of causing cell death, had little or no effect on bFGF stimulation of MAPK activity. Thus, in relation to JNK and MAPK, TNFα and bFGF receptors independently regulate the activity of these two sequential protein kinase pathways in L929 cells.

Inducible expression of inhibitory and activated Ras influences apoptosis Ras activation is required for many of the phenotypic responses resulting from the activation of tyrosine kinases. Signaling by the bFGF receptor involves several different effector pathways including Ras activation. To test the involvement of Ras in the bFGF protective response, the Lac Switch inducible expression system was used to control the expression of inhibitory N17 Ras and constitutively activated V12 Ras in L929 cells. Expression of N17 Ras significantly blunted bFGF stimulation of MAPK, but had no effect on TNF stimulation of JNK. With two independent clones, expression of V12 Ras did not constitutively activate the MAPK pathway, but did appear to enhance bFGF stimulation of MAPK. V12 Ras expression also had no effect on TNFα stimulation of JNK activity. Similar results were found with independent L929 cell clones indicating the responses were the result of specific mutant Ras expression.

Expression of N 17 Ras did not affect TNFα induced apoptosis of L929 cells; N 17 Ras did, however, markedly inhibit the ability of bFGF to protect cells against TNFα-mediated cell death. These findings indicated that functional Ras signaling is not required for the TNF α-induced apoptotic response, but is required for the protective action of bFGF. Strikingly, constitutively activated V12 Ras has markedly enhanced TNFα-stimulated apoptosis, but had little or no effect on the apoptotic index of L929 cells in the absence of TNFA. This observation indicates that V12 Ras is functional in L929 cells, despite the fact MAPK is not constitutively activated in this cell line and implies that activated Ras likely regulates pathways in addition to MAPK that are involved in apoptosis. Co-stimulation with bFGF and TNFα resulted in a diminished apoptotic response relative to TNFα alone in V12 Ras expressing cells, indicating that bFGF pathways required for protection against TNFα stimulated cell death were functional in these cells. Thus, inhibitory Ras expression prevented bFGF protective responses and activated Ras enhanced TNFα killing. The results suggest multiple Ras-dependent events are involved in controlling apoptosis and the role of Ras signaling can be either positive or negative in regulating the phenotypic response to cytokines such as TNFα.

Inhibition of MEK and MAPK stimulation prevents bFGF protection from apoptosis The Parke-Davis compound, PD #098059 inhibits the dual specificity protein kinase, MEK-1, which specifically activates p42/p44 MAPK (Alesssi, D. R., Cuenda, A., Cohen, P., Dudley, D. T., and Saltiel, A. R. (1995) J. BioL Chem. 270:27489-27494). PD #098059 did not inhibit JNK kinase or the activation of JNK. Pretreatment of L929 cells with PD #098059 inhibited bFGF stimulation of MAPK activity. The PD #098059 compound had no effect on TNFα-mediated apoptosis but inhibited the protective action of bFGF. Thus, MEK activation of MAPK is required for bFGF protection against TNFα-mediated apoptosis. Interestingly, the phosphatidylinositol 3-kinase inhibitor, wortmannin, did not influence the cell death response to TNFα nor did it inhibit the protective response to bFGF. Treatment of L929 cells with wortmannin had no effect on the ability of bFGF to stimulate MAPK activity. Apparently, phosphatidylinositol 3-kinase activity is not required for the action of either TNFα or bFGF on the control of the cell death program L929 cells.

TNFα induces apoptosis of L929 cells and bFGF is protective against this cell death response. Our results indicate that the activation of JNK in response to TNFα stimulation of L929 cells is not sufficient for the induction of cell death. TNFα maximally stimulates JNK activity in the presence of bFGF concentrations that are capable of protecting against cell death. Signals in addition to JNK activation must be involved in the TNFα-mediated death response. The bFGF protective response was only partial in that not all the cells were prevented from dying in response to TNFα treatment. This may, in part, be related to cell cycle dependent signaling by TNFα and bFGF; the L929 cells used in these studies were asynchronous so that we can not rule out this possibility. Our findings also demonstrate that Ras is involved in integrating responses that control apoptosis. Expression of activated or inhibitory Ras influences TNFα killing of L929 cells. The mechanism for enhanced TNFα killing of L929 cells resulting from V12 Ras expression is unclear, although it has been observed in C3H mouse fibroblasts as well (Fernandez, A., et al. (1994) Oncogene 9:2009-2017). It may involve an alteration in the expression of specific genes such as c-Jun, c-Fos and c-Myc which appear to be involved in both growth and apoptotic responses (Westwick, J., et al. (1994) J. Biol. Chem. 269:26396-6401; Pulverer, B. J., et al. (1991) Nature 353:670-674; Seth, A., et al. (1991) J. Biol. Chem. 266:23521-23524; Evan, G. I., et al. (1992) Cell 69:119-128; Gupta, S., Seth, A., and Davis, R. J. (1993) Proc. Natl. Acad. Sci. (USA) 90:3216-3220; Klefstrom, J., et al. (1994) EMBO J. 13:5442-5450; Shi, Y., et al (1992) Science 257:212-214; Janicke, R. U., Lee, F. H. H., and Porter, A. G. (1994) Mol. Cell. Biol. 14:5661-5670; (Harrington, E. A., et al. (1994) EMBO J. 13:3286-3295). In contrast, the effect of inhibitory N17 Ras appears to primarily be the inhibition of MAPK activation in response to bFGF. This finding is substantiated by the loss of bFGF protection against TNFα -mediated apoptosis by the MEK inhibitor PD #098059. Studies using the fungal metabolite, wortmannin, demonstrated that hosphatidylinositol 3-kinase was not involved in bFGF protection against apoptosis in L929 cells.

Recently, it was demonstrated using PC12 cells that the JNK pathway was involved in mediating apoptosis in response to serum deprivation and that activation of the MAPK pathway was protective against serum deprivation (Xia, Z., et al.(1995) Science 270:1326-1331). Phosphatidylinositol 3-kinase activity has also been reported to be necessary to protect PC12 cells from serum deprivation induced apoptosis (Yao, R., and Gooper, G. M. (1995) Science 267:2003-2006). Interestingly, the expression of N17 Ras protected PC12 cells from nerve growth factor withdrawal induced apoptosis (Ferrari, G., and Greene, L. A. (1994) EMBO J. 13:5922-5928). The findings indicated that N17 Ras maintained PC12 cells in a quiescent state that allowed them to survive in the absence of trophic factors. Removal of trophic factors from PC12 cells appeared to induce an aberrant proliferative response that resulted in apoptosis. Our findings using N17 Ras expression in L929 cells contrast with those in PC12 cells. TNF induced apoptosis in growing L929 cells, N17 Ras expression did not affect the apoptotic response, while V12 Ras expression significantly enhanced apoptosis. Thus, the involvement of Ras dependent signaling on apoptotic responses of cycling versus quiescent cells may be quite different.

In human B cells, crosslinking of surface IgM stimulated a host of signaling pathways including MAPK but not JNK and resulted in apoptosis (Sakata, N., Patel, H., Aruffo, A., Johnson, G. L., and Gelfand, E. W. (1995) J. Biol. Chem. 270:30823-30828). CD40, a member of the TNF receptor family, activated JNK while rescuing B cells from anti-IgM mediated apoptosis (Sakata, N., Patel, H., Aruffo, A., Johnson, G. L., and Gelfand, E. W. (1995) J. Biol. Chem. 270:30823-30828). Thus, in human B cells MAPK activation is insufficient to protect against apoptosis and signals including the stimulation of JNK are generated during a protective response. Clearly, the integration of multiple signals appears to be required for apoptosis.

The overlap of signals involved in committing cells to growth or apoptosis is also evident in many transformed cell types. Tumors frequently have a high growth rate, but also a high apoptotic index (Evan, G. I., et al. (1992) Cell 69:119-128; Fanidi, A., Harrington, E. A., and Evan, G. I. (1992) Nature 359:554-556). The growth rate is simply greater than the apoptotic rate so that the net result is tumor expansion. In addition, transformed cells frequently have selected mutations and growth factor autocrine loops to inhibit apoptosis. For example, Ras function has been shown to be involved in both transformation and protection against apoptosis in Bcr-Abl transformed cells (Cortey, D., Kadlec, L., and Pendergast, A. M. (1995) Mol. Cell. Biol. 15:5531-5541; Goga, A., et al. (1995) Cell 82:981-988).

Cumulatively, the results in different cell types indicate that it is the integration of multiple signals from cytokines and growth factors that determines the commitment to apoptosis. Similarly, integration of multiple signals and not a single dominant signaling pathway is likely involved in the commitment to growth or differentiation. The requirement for signal integration may allow for specific checkpoints so that cells do not die or grow inappropriately. In this regard, cell systems where specific cytokines or growth factors are added or removed are most relevant in defining the integration of signals controlling growth versus death.

The implication of our findings is that it should be possible to define signal pathways and their integration that controls apoptosis in specific cell types. As these findings are further defined it will be possible to develop strategies to selectively induce a cell type-specific apoptotic response. Development of gene therapy, cytokine and drug treatments may be possible to selectively promote the death of undesirable cell populations in animals.

### EXAMPLE 32

This example demonstates that MEK kinase 1 (MEKK1), a 196 kDa protein kinase, functions to integrate proteases and signal transduction pathways involved in the regulation of apoptosis. Cleavage of mouse MEKK1 at Asp⁸⁷⁴ generates a 91 kDa kinase fragment and a 113 kDa NH₂-terminal fragment. The kinase fragment of MEKK1 induces apoptosis. Cleavage of MEKK1 and apoptosis are inhibited by p35 and CrmA, viral inhibitors of the ICE/FLICE proteases that commit cells to apoptosis. Mutation of the MEKK1 sequence ⁸⁷¹DTVD⁸⁷⁴ (SEQ ID NO: 7), a cleavage site for CCP32-like proteases, to alanines inhibited proteolysis of MEKK1 and apoptosis induced by overexpression of MEKK1. Inhibition of MEKK1 proteolysis inhibited apoptosis but did not block MEKK1 stimulation of c-Jun kinase activity, indicating that c-Jun kinase activation was not sufficient for apoptosis. During the apoptotic response to UV irradiation, cisplatin, etoposide and mitomycin C, MEKK1 undergoes a phosphorylation-dependent activation followed by its proteolysis. These results show that MEKK1 activation and cleavage occurs in response to genotoxic agents and the activated kinase fragment functions to commit cells to apoptosis.

Publications referred to in these examples are abbreviated using the first author's name and the year of publication. A list of the full citation of each publication referred to in this example is provided at the end of the example.

Apoptosis or programmed cell death is a physiological process important in differentiation and tissue modelling (Williams and Smith, 1993; Steller, 1995). Apoptosis can be triggered by many different stimuli including growth factor deprivation (Xia et al., 1995; Park et al., 1996), exposure of specific cell types to cytokines such as TNFa and Fas ligand (Vandenabeele et al., 1995; Kägi et al., 1994; Lowin et al., 1994), virus infection (Esolen et al., 1995; Hinshaw et al., 1994; Terai et al., 1991; Tyler et al., 1995), and DNA damaging agents including irradiation and chemicals that induce DNA adducts (Canman and Kastan, 1996). Proteases of the ICE/FLICE family are activated during the apoptotic response that cleave specific protein substrates resulting in an irreversible commitment to cell death. Several ICE/FLICE substrates have been identified including poly (ADP-ribose) polymerase (Lazebnik et al., 1994), U1 small nuclear ribonucleoprotein (Casciola-Rosen et al., 1994), lamin (Lazebnik et al., 1995), D4-GDI (Na et al., 1996), fodrin (Cryns et al., 1996), protein kinase Cδ (Emoto et al., 1995), sterol regulatory element binding protein (Wang et al., 1996), retinoblastoma protein (An and Dou, 1996), DNA-dependent protein kinase (Casciola-Rosen et al., 1995), and the proteases themselves (Orth et al., 1996).

Two ICE-like protease activities appear necessary for the apoptotic response, each with a specific substrate selectivity. ICE-like proteases such as Ced-3 have a specificity for proteins encoding the four amino acid sequence YVAD (SEQ ID NO: 10) (Howard et al., 1991) while CPP32-like proteases have a preference for the sequence DEVD (SEQ ID NO: 11) (Nicholson et al., 1995). Both groups of proteases cleave at the terminal aspartic acid residue of the recognition sequence. Several viruses encode proteins that are specific inhibitors of the ICE/FLICE proteases. Most notably CrmA is a poxvirus protein that inhibits ICE-like proteases, and p35 is a baculovirus protein that has broad inhibitory activity to ICE/FLICE-like proteases (Fraser and Evan, 1996; Clem et al., 1996). Expression of CrmA and p35 inhibit the apoptotic response to many different stimuli demonstrating the requirement of ICE/FLICE proteases during programmed cell death (Beidler et al., 1996; Los et al., 1995).

In addition to ICE/FLICE proteases, it is becoming increasingly clear that signal transduction pathways involving specific protein kinases are involved in mediating apoptosis. Specifically, the c-Jun kinases (JNK) and p38 kinases have been proposed to mediate apoptosis (Verheij et al., 1996; Xia et al., 1995). However, a number of reports have challenged the notion that activation of JNKs and/or p38 is sufficient to induce apoptosis (Lassignal Johnson et al., 1996; Tsubata et al., 1993; Liu et al., 1996a; Juo et al., 1997; Liu et al., 1996b; Park et al., 1996). It appears thus that other signal pathways are required for apoptosis. However, the integration and balance of the JNK and p38 pathways probably does contribute to the commitment to apoptosis (Xia et al., 1995; Gardner and Johnson, 1996).

Several protein serine-threonine kinases referred to as MEK kinases (MEKKs) have been cloned that are members of sequential protein kinase pathways regulating MAP kinases including the c-Jun kinases and ERKs [(Lange-Carter et al., 1993; Lange-Carter and Johnson, 1994; Xu et al., 1996; Blank et al., 1996)]. In our hands, MEKKs do not significantly activate p38 kinases. Of the four MEKK members we have characterized, MEKK1 has been found to have the unique property of being a strong stimulator of apoptosis (Lassignal Johnson et al., 1996; Xia et al., 1995). The other MEKKs, even though they all activate c-Jun kinases and ERKs to different levels, do not induce apoptosis, suggesting MEKK1 has unique substrates that mediate the death response. The kinase domain of MEKK1 is only 50% conserved relative to the kinase domains of MEKK2, 3 and 4, consistent with MEKK1 having unique substrate recognition properties and catalytic activity involved in mediating the apoptotic response. MEKK1 is a 196 kDa protein that encodes a protease cleavage sequence for CPP32-like proteases. None of the other MEKKs or known kinases that regulate MAPK pathways have a consensus ICE/FLICE cleavage site. We demonstrate in this example that MEKK1 is a substrate for proteases inhibited by the p35 baculovirus protein. When the kinase domain is released from the holo-MEKK1 protein it functions as a physiological activator of apoptosis. UV irradiation and DNA damaging chemicals activate MEKK1 kinase activity and induce its proteolytic cleavage indicating that MEKK1 contributes to apoptosis in response to environmental stresses.

Materials and Methods for this Example:

Cells

Human embryonal kidney 293 cells (HEK293) stably expressing the EBNA-1 protein from Epstein-Barr virus (Invitrogen) were grown in Dulbecco's modified Eagle's medium (DMEM) supplemented with 100 U/ml penicillin/streptomycin and containing 10% bovine calf serum (BCS). The cells were transfected using lipofectamine (Gibco).

Plasmids

The full length cDNA encoding mouse MEKK1 was modified by addition of the HA-tag sequence (MGYPYDVDYAS) (SEQ ID NO: 12) at its NH₂-terminus and inserted into the expression plasmid pCEP4 (Invitrogen), resulting in plasmid MEKK1.cp4. The MEKK1 sequences DTVD (amino acids 871-874) and DEVE (amino acids 857-860) in MEKK1.cp4 were substituted with alanines using a PCR strategy. The resulting plasmids were named DTVD_A.cp4 and DEVE_A.cp4. The cDNAs for CrmA (Pickup et al., 1986), p35 (Cartier et al., 1994), JNK1-APF (Dérijard et al., 1994) and JNK2-APF (Kallunki et al., 1994) were subcloned in pCEP4 in which the hygromycin resistance gene had been removed, resulting in plasmids CrmA.cp_, p35.cp_, JNK1_APF.cp and JNK2_APF.cp_. Plasmid pCDNA_{—}3.cp4 is the result of the ligation of pCEP4 and pCDNA-3.

In vitro kinase assays

Lysis buffer (70 mM β-glycerophosphate, 1 mM EGTA, 100 μM Na₃VO₄, 1 mM DTT, 2 mM MgCl₂, 0.5% Triton-X100, 20 μg/ml aprotinin) was added to cells 15-24 hours after transfection. Cellular debris was removed by centrifugation at 8,000xg for 5 min. Protein concentration was normalized by Bradford assay using BSA as standard.

c-Jun Kinase

c-Jun kinase (JNK) activity was measured using a solid phase kinase assay in which glutathione S-transferase-c-Jun(1-79) (GST-Jun) bound to glutathione-Sepharose 4B beads was used to affinity-purify JNK from cell lysates as described (Gardner and Johnson, 1996; Hibi et al., 1993). Alternatively, JNKI or JNK2 were immunoprecipitated with isoform specific antibodies (Santa Cruz Biotechnology) and GST-Jun used as substrate in an in vitro kinase assay (Hibi et al., 1993). Quantitation of the phosphorylation of GST-Jun was performed with a Phosphorlmager.

ERK

ERK2 was immunoprecipitated as described above for the JNK isoforms using the ERK2 (C-14) antibody (Santa Cruz Biotechnology). The beads were washed twice with 1 ml lysis buffer and twice with 1 ml lysis buffer without Triton-X100. Thirty-five μl of the last wash was left in the tube and mixed with 20 μl of kinase 2X mix (50 mM β-glycerophosphate, 100 μM Na₃VO₄, 20 mM MgCl₂, 200 μM ATP, 1 μCi/μl γ³²P-ATP, 400 μM EGF receptor peptide 662-681, 100 μg/μl IP-20, 2 mM EGTA), incubated 20 min at 20° C. and spotted on P81 Whatman paper. The samples were washed thrice for 5 min each in 75 mM phosphoric acid and once for 2 min in acetone, air-dried, and their radioactivity determined in a β counter.

SEK1 K→M Phosphorylation

MEKK1 was immunoprecipitated from cell lysates (200-500 μg) with the antibodies raised against specific sequences of MEKK1 or the 12CA5 antibody that recognizes the HA-tag sequence. The immunoprecipitates were used in an in vitro kinase assay with recombinant kinase inactive SEK1 (SEK1 K→M) as previously described (Blank et al., 1996).

MEKK1 Staining and Terminal-deoxy-transferase (TdT)-mediated incorporation of Fluoresecent dUTP

Cells were grown on glass coverslips and transfected using lipofectamine. Two days after transfection, the medium was removed and the cells were fixed in 2% paraformaldehyde, 3% sucrose in phosphate buffered saline (PBS) for 10 min at room temperature. Following three washes with PBS, the cells were permeabilized for 10 min with 2% Triton-X100 in PBS. After three PBS washes, the cells were blocked with filtered cultured medium for 15 min. The coverslips were then incubated 1 hour in TdT reaction mix (200 mM potassium cacodylate, 25 mM Tris.HCl, pH 6.6, 250 μg/ml BSA, 5 mM CoCl₂, 0.25 U/μl TdT [Boehringer], 10 μM biotin-dUTP [Boehringer]) at 37° C. in a humidifed atmosphere. After three washes in PBS, the coverslips were incubated for 1 hour at room temperature with a 1/500 dilution in filtered culture medium of an affinity purified rabbit antisera directed at the peptide DRPPSRELLKHPVFR of mouse MEKK1 (amino acids 1476-1490) (Lange-Carter et al., 1993). The coverslips were then washed 6× over a 30 min period with PBS and incubated 1 hour at room temperature with a 1/1000 dilution in filtered culture medium of a donkey anti-rabbit, Cy³-conjugated, antibody (Jackson Immunological) mixed with 5 μg/ml streptavidin conjugated with FITC (Jackson Immunological). The coverslips were washed 6× with PBS and incubated overnight in PBS before being mounted in 20 mg/ml o-phenyldiamine-diHCl (Sigma) in 0.1 M Tris pH 8.5, 90% glycerol. Images were taken using a Leica DMRXA microscope and analyzed with the SlideBook v2.0 software (Intelligent Imaging Innovations, Denver). The subcellular localization of endogenous MEKK1 observed with the anti-COOH-terminal MEKK1 antibody was identical to that observed with a second antibody recognizing the NH₂-terminal portion of the MEKK1 protein.

Immunoblots

200-400 μg cell lysate protein was subjected to SDS-9% PAGE and transferred to nitrocellulose membranes. Blots were performed exactly as described (Widmann et al., 1995). To detect HA-tagged proteins, the mouse monoclonal antibody 12CA5 (Babco) was used as the primary antibody, followed by a rabbit anti-mouse antibody (Cappel). HRP-conjugated protein A at a 1/5000 dilution (Zymed) and ¹²⁵I-protein A at a 1/500 dilution (Dupont NEN) were then used for enhanced chemiluminescence (ECL) detection and for quantification using the phosphorimager. To detect MEKK1, 3 different ploclonal antisera were used as primary antibodies, followed by ECL detection using HRP-protein A (see above). These sera were generated by injecting rabbits with GST proteins fused with different portions of the MEKK1 protein.

PP-2A treatment

MEKK1 was immunoprecipitated from cell lysates (200-500 μg) using the 96-001 (NH₂) antisera, washed twice with 1 ml extraction buffer (EB) [1% Triton-X100; 10 mM Tris pH 7.4; 50 mM NaCl; 50 mM NaF; 5 mM EDTA], twice with 1 ml TC (50 mM Tris pH 7.0; 0.1 mM CaCl₂) and once with 1 ml TC containing 60 mM β-mercaptoethanol, 1 mM MgCl₂. 35 μl of the last wash was left in the tube and 0.5 U of PP-2A (Upstate Biotechnology) was added for 30-45 min. The phosphatase reaction was terminated by adding 1 μl of 200 mM Na₃VO₄. For in vitro kinase assay, the immunoprecipitates were washed three more times with 1 ml PAN (10 mM PIPES; 100 mM NaCl; 20 μg/ml aprotinin) before being mixed with the SEK1 K(M substrate and γ³²P-ATP.

Results

Expression of the 196 kDa MEKK1 Protein by Gene Transfection Induces Apoptosis.

Expression of the 37 kDa kinase domain of MEKK1 (ΔMEKK1) induces cell death by apoptosis (Lassignal Johnson et al., 1996; Xia et al., 1995). To assess whether the full length protein had the same effect, HEK293 cells were transfected with a plasmid encoding the mouse MEKK1 and stained 2 days later for MEKK1 expression using an antibody directed at the COOH-terminus of the protein. To monitor cell death, DNA fragmentation, a feature often associated with apoptosis, was measured by terminal-deoxy-transferase-mediated incoporation of fluorescent dUTP. Alarge proportion of HEK293 cells expressing MEKK1 had fragmentated DNA. The MEKK1 expressing cells characteristically rounded up and began to lift off the coverslips. MEKK1 also induced chromatin condensation and the nuclei in these cells often dissociated from the surrounding cytoplasm. Quantitation of cells exhibiting DNA fragmentation and cells expressing MEKK1 revealed that about 30% of MEKK1-expressing cells were apoptotic after 48 hr. This is an underestimate because the apoptotic cells eventually detach from the coverslips and often loose their nucleus. Thus, expression of the 196 kDa MEKK1 protein by gene transfection induced cell death characteristic of apoptosis similar to that observed for the 37 kDa kinase domain. The kinase activity of MEKK1 is required for the induction of cell death (Lassignal Johnson et al., 1996).

MEKK1-induced DNA Fragmentation is inhibited by p35 and CrmA.

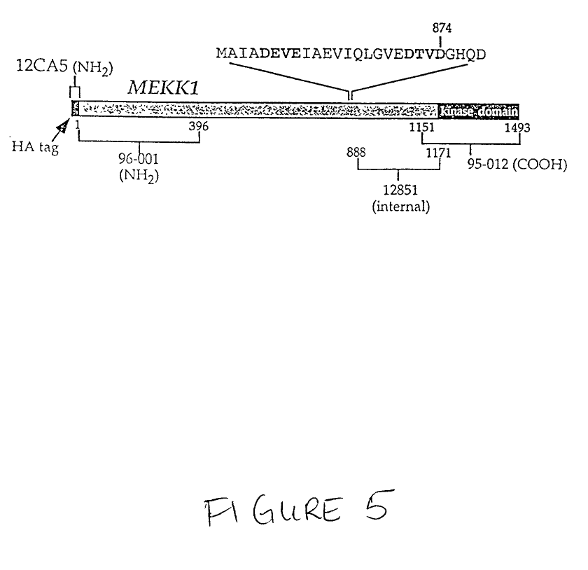

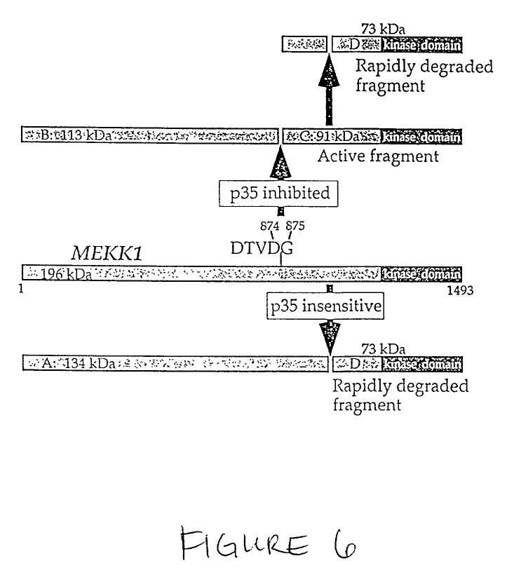

Inhibition of cysteine proteases of the ICE family by the baculovirus p35 protein or by the poxvirus CrmA protein has been shown to protect cells from apoptosis in response to diverse stimuli (Beidler et al., 1996). Cotransfection of HEK293 cells with MEKK1 and p35 inhibited the DNA fragmentation seen with expression of MEKK1 alone. Cotransfection of MEKK1 with CrmA also inhibited DNA fragmentation, but to a lesser extent. While only about 5% of the cells cotransfected with MEKK1 and p35 showed some DNA fragmentation, this proportion increased to about 15% in MEKK1- and CrmA-cotransfected cells. A small area of fragmented DNA was typically seen in the nucleus of these cells. Thus CrmA appears to be less efficient in protecting cells from MEKKl-induced apoptosis. Interestingly, co-expression of inhibitory mutants of the c-Jun kinases (JNK1-APF and JNK2-APF) with MEKK1 had no or only modest effects on MEKK1-mediated apoptosis. JNK1-APF expression had no effect and JNK2-APF had only a 30% dimunition of apoptotic cells induced by MEKK1 expression. FIG. 3 shows quantitation of the percentage of MEKK1-transfected cells in the presence or in the absence of the caspase inhibitors CrmA or p3 5, that showed DNA fragmentation as an indication of apoptosis.

CrmA and p35 Inhibit Cleavage of the 196 kDa MEKK1 Protein and Generation of an Activated Kinase Fragment.

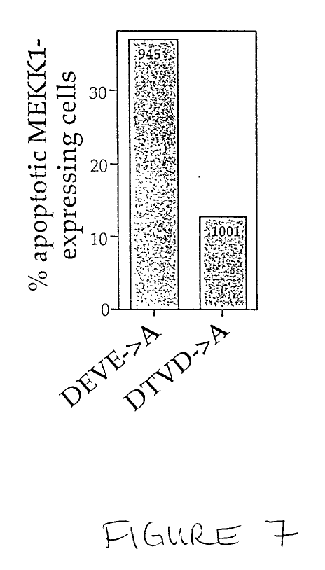

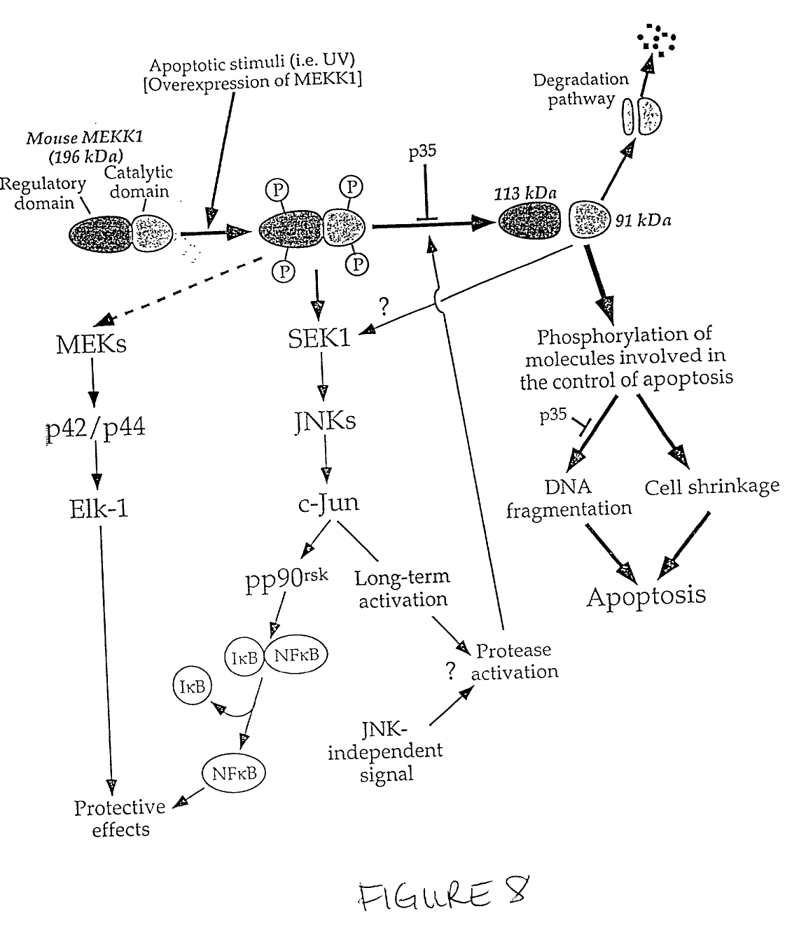

When MEKK1 was expressed by transfection of HEK293 cells, two additional immunoreactive polypeptides besides the full length protein (named A and B, left panel, FIG. 4), were detected by Western blot using an antibody directed to the HA tag of MEKK1 (12CA5 antibody). The 12CA5 antibody recognizes the first 11 amino acids at the NH₂-terminus of the tagged MEKK1 protein, indicating that fragments A and B must be the result of proteolysis of the full length MEKK1 protein and cannot have arisen from other potential translation sites. When an antibody directed at th(COOH-terminus of MEKK1 was used (95-012 antibody), additional immunoreactive fragments were also detected (FIG. 4, right panel). Based on their apparent molecular weight, two of these fragments, named C and D, are the corresponding moieties of the cleavage products B and A, respectively. It is also important to note that the proteolytic activity can generate fragment D from fragment C. Based on its behaviour in the SDS gel, the band marked with an asterisk in FIG. 4 is probably a dimer of D. The observation that MEKK1 can be proteolyzed to very specific fragments prompted us to determine whether p35 or CrmA could inhibit the generation of fragments A, B, C and D. FIG. 4 shows that p35 almost totally, and CrmA partially, inhibited the appearance of fragments B and C. Quantitation of the fragments in 6 independent experiments revealed that CrmA and p35, while leaving the amount of fragment A unchanged, diminished the amount of fragment B by 50% and 90%, respectively. This indicates that these protease inhibitors prevented the formation of fragments B and C, but had no effect on the proteolytic activity that cleaves MEKK1 into fragment A. Since the cleavage of MEKK1 into fragment A was unaffected by CrmA and p35, it was suprising to find that the amount of fragment D, the corresponding moiety of fragment A, was reduced in the presence of the inhibitors (FIG. 4). However, because the amounts of fragments A and B formed in MEKK1-transfected cells are not significantly different from one another, the observation that there is far less fragment D than fragment C (FIG. 4, MEKK1 lane, right panel) suggests that fragment D may be unstable and rapidly degraded. Moreover, since fragment D can be derived from fragment C, blocking the generation of fragment C will result in less fragment D. Neither JNK1-APF nor JNK2-APF expression influenced the generation of MEKK1 fragments, suggesting that blunting the activation of the JNK1/JNK2 pathways had little effect on the proteolysis of the MEKK1 protein.

To determine whether the cleavage of MEKK1 into fragments A, B, C and D had any effect on the kinase activity of MEKK1, lysates from cells transfected with HA-tagged MEKK1 alone or in combination with CrmA or p35 were used for immunoprecipitation with the 12CA5 HA antibody or with an antibody specific for the COOH-terminal moiety of MEKK1 (antibody 95-012). The immunoprecipitates were then incubated with a MEKK1 substrate (SEK1 K(M) and γ³²P-ATP. When the full length MEKK1 protein was immunoprecipitated by the 12CA5 antibody it had measureable autophosphorylation and activity towards SEK1. When MEKK1 was immunoprecipitated with the COOH-terminal 95-012 antibody, a stronger SEK1 phosphorylation signal was detected. Since the full length MEKK1 protein and fragments C and D are immunoprecipitated with similar efficiency, the increased phosphorylation of SEK1 was due to the presence of fragments C and D in the immunoprecipitates. This phosphorylation was reduced in the presence of CrmA. In the presence of p35, phosphorylation of SEK1 reached the same level of phosphorylation observed when the 12CA5 antibody was used, that is the basal level of phosphorylation induced by the full length MEKK1. Phosphorylation of fragments C and D was also detected in 95-012 immunoprecipitates. This phosphorylation was reduced by CrmA and almost completely abolished by p35, as expected from the effect of these inhibitors on the generation of fragments C and D (See FIG. 4). In summary, there is a strong correlation between MEKK1-induced apoptosis and the generation of MEKK1-derived cleavage products that have a stronger kinase activity than the full length protein. This suggests that proteolysis of MEKK1 is involved in the cell death response.

p35 Inhibited Cleavage Occurs at Position Asp⁸⁷⁴ in the Mouse MEKK1 Protein.

The p35-inhibited cleavage of MEKK1 generates a COOH-terminal fragment of about 90 kDa and a NH₂-terminal fragment of about 110 kDa (see FIG. 4), indicating that the cleavage occurs between residues 820-900. Two tetrapeptide sequences that are found in this region of MEKK1 closely ressemble the CPP32 cleavage site, DEVD (SEQ ID NO: 11) (Nicholson et al., 1995). These sequences are ⁸⁵⁷DEVE⁸⁶⁰ (SEQ ID NO: 6) and ⁸⁷¹DTVD⁸⁷⁴ (SEQ ID NO: 7) (see FIG. 5). The proteases inhibited by p35 have been shown to be cysteine proteases cleaving after the aspartic acid residue in the fourth position of the consensus cleavage sequence (Nicholson et al., 1995; Howard et al., 1991) and, therefore only the DTVD (SEQ ID NO: 7) sequence should be a cleavage site for the CPP32-like protease. Two mutants were generated that have either the DEVE (SEQ ID NO: 6) or the DTVD (SEQ ID NO: 7) sequence replaced with alanine residues (see FIG. 5). These mutants were transfected into HEK293 cells and the presence of MEKK1 and MEKK1-derived fragments were detected by immunoblot analysis using three MEKK1-specific antibodies. When transfected into HEK293 cells, the DEVE→A mutant, like the wild-type protein, was cleaved into fragments A, B, C and D. In contrast, the DTVD→A mutant was only cleaved into fragments A and D. Thus, fragments B and C are not generated in cells expressing the DTVD→A mutant or in cells expressing MEKK1 and p35. This indicates that the p35-inhibited cleavage occurs at position Asp⁸⁷⁴ in the mouse MEKK1 sequence.

The kinase activity of the mutants expressed in HEK293 cells was determined. Immunoprecipitating full length 196 kDa MEKK1 or mutant MEKK1 proteins with the 12CA5 antibody resulted in similar SEK1 phosphorylating activities. However, when the antibodies directed towards the COOH-terminus of the protein were used, SEK1 phosphorylating activity was reduced in DTVD→A expressing cells as compared to the activity found in wild-type or DEVE→A expressing cells. The reduced kinase activity was comparable to the basal SEK1 phosphorylating activity observed when the full length proteins were immunoprecipitated. Thus, the mutant DTVD→A MEKK1 protein has a low but measureable kinase activity towards SEK1 because fragment C is not generated. The same result was observed when the cleavage of MEKK1 into fragments B and C was inhibited by p35 expression.

Based on the results described above, FIG. 6 describes a model of the MEKK1 cleavage events occuring in transfected cells. In this model, overexpression of MEKK1 induces deregulated cleavage events generating two sets of fragments (A and D; B and C). Fragment C encoding the catalytic domain of MEKK1 has a stronger kinase activity than the full length protein. Proteases of the ICE/FLICE family are responsible for the cleavage of MEKK1 into fragments B and C because this cleavage can be inhibited by p35 and CrmA. Mutagenesis experiments revealed that the cleavage site generating fragments B and C is DTVD⁸⁷⁴ (SEQ ID NO: 7). Fragment C can be further processed into a smaller polypeptide (fragment D) which may be rapidly degraded. It is possible that the proteolytic activity which generates fragment D is part of a regulatory mechanism involved in the termination of the response induced by cleavage of MEKK1 into the active fragment C.

The DTVD→A Mutant has a Reduced Ability to Promote DNA fragmentation in HEK293 Cells.

We next determined whether the DTVD→A mutant induces DNA fragmentation when expressed in HEK293 cells. Expression of the DEVE→A mutant or the wild-type MEKK1 protein induced DNA fragmentation. In contrast, cells expressing the DTVD→A mutant MEKK1 protein showed little DNA fragmentation. As shown in FIG. 7, quantitation of the response revealed that the number of DTVD→A expressing cells that showed some DNA fragmentation was reduced by 65% compared to the cells transfected with wild-type MEKK1 or the DEVE→A mutant. This indicates that cleavage of MEKK1 into fragments B and C is required to induce cell death.

p35 Inhibits ΔMEKK1-induced Apoptosis.

The 37 kDa kinase domain of MEKK1 (ΔMEKK1) is a strong inducer of apoptosis (Lassignal Johnson et al., 1996; Xia et al., 1995). Since p35 inhibits programmed cell death induced by most, if not all, apoptotic stimuli (Clem et al., 1996), we determined whether this inhibitor could also block ΔMEKK1-induced apoptosis. ΔMEKK1 induced DNA fragmentation when expressed in HEK293 cells. This effect was inhibited by co-expression of p35. Quantitation showed that 40% of cells expressing ΔMEKK1 showed DNA breaks; co-expression of p35 and ΔMEKK1 reduced this number to 10%. The number of ΔMEKK1-expressing cells appeared to be increased when p35 was present, suggesting that less cell death occured when ΔMEKK1 and p35 were co-expressed. Even if the co-transfected cells showed less DNA fragmentation compared to the cells transfected with ΔMEKK1 alone, they were clearly affected by the expression of ΔMEKK1 and were rounded and most showed some membrane blebbing. This differed from the effect of p35 in full length MEKK1-transfected cells, where the inhibitor appeared to better protect the cells from DNA fragmentation and obvious morphological changes, the predicted result if cleavage of MEKK1 results in the release of an activated kinase domain. These results indicate that p35 inhibits at least two steps in the pathway leading to MEKK1-induced apoptosis, the cleavage of MEKK1 into an active kinase fragment and events downstream of the MEKK1 cleavage that most likey involves a protease step that is influenced by MEKK1.

Activation of the ERK and the JNK Pathways is not Correlated with MEKK1 -induced DNA Fragmentation.

MEKK1 activates the ERK and JNK pathways (Xu et al., 1996). Since activation of the JNK pathway has been proposed to induce apoptosis (Verheij et al., 1996), we determined whether inhibitory mutants of JNK1 or JNK2 (JNK 1 -APF and JNK2-APF, respectively) could prevent MEKK1-induced DNA fragmentation. While JNKI-APF had no protective effect, JNK2-APF slightly (by about 30%) reduced the number of MEKK1-expressing apoptotic cells. The competitve inhibitory JNK mutants had no effect on the generation of any cleavage products, indicating that the JNK2-APF-mediated inhibition of MEKK1-induced DNA fragmentation is not related to the cleavage of MEKK1. Activation of ERK2 or the JNKs by MEKK1 was unaffected by the co-expression of JNK1-APF, JNK2-APF, p35 or CrmA. When specific JNK isoforms were immunoprecipitated, only JNK1-APF and JNK2-APF partially inhibited JNK1 and JNK2 activity, respectively. The partial inhibition may be due to cross-reactivity of the antibodies used (Gupta et al., 1996). The DEVE→A and DTVD→A mutants activated JNK to the same level as wild type MEKK1. Transfection of MEKK1 in HEK293 cells did not activate the p38 kinase. Cumulatively, these results show that in conditions where MEKK1-induced DNA fragmentation is inhibited (i.e. when p35 is cotransfected with MEKK1 or when the DTVD→A mutant is expressed), the ERK and the JNK pathways are still activated to an extent similar to that found in MEKK1-transfected cells. This indicates that neither the ERK nor the JNK pathways are sufficient to promote or inhibit the cell death pathway induced by cleavage of MEKK1.

UV Irradiation of HEK293 Cells Induces a Rapid Phosphorylation and Subsequent Cleavage of the Endogenous MEKK1 Protein.

To determine the relevance of our findings in a more physiological situation, we examined the regulation of endogenous MEKK1 in response to UV irradiation, a stress stimulus that induces an apoptotic response. In HEK293 cells, three different antisera directed at the mouse MEKK1 protein recognized the 196 kDa MEKK1 protein. Several additional nonspecific immunoreactive protein bands were also detected. When cells were treated with UV irradiation (100 J/m²) and incubated for 24 hours in low serum media, the full length MEKK1 protein was no longer detected. Since, we have determined that the half-life of MEKK1 is greater than 24 hours, this result indicates that UV induces a cleavage of the MEKK1 protein. UV irradiation also induced the appearance of new immunoreactive species, the majority of which have molecular weights ranging from about 100 kDa to about 120 kDa. These polypeptides appear thus to be MEKK1-derived fragments generated following MEKK1 proteolysis. The results indicate that UV induces cleavage of the endogenous MEKK1 protein in HEK293 cells.

A time course was performed to determine the effects of UV irradiation on the endogenous MEKK1 protein, activation of the JNK pathway and the extent of apoptosis resulting from the exposure of the cells to a stress stimulus. 15 min after UV irradiation, an MEKK1 species is generated that was upward gel-shifted compared to the MEKK1 species detected before exposure to UV irradiation. One hour after irradiation, most of the full length MEKK1 protein was upward gel-shifted. Eight hours after irradiation, the amount of the gel-shifted MEKK1 started to decrease and 20 hours after UV treatment only a trace amount of full length MEKK1 was detected. The MEKK1 fragment detected by the 96-001 (NH2) antibody was barely seen in the control condition. After 1 hour, however, there was a clear increase in the production of the MEKK1 fragment which reached a maximum 8 hours after UV irradiation. In MEKK1-transfected cells, both the shifted and non-shifted forms of full length MEKK1 were detected. To determine whether the upward gel shift of MEKK1 was due to phosphorylation, lysates of MEKK1-transfected cells were immunoprecipitated with the 12CA5 antibody and incubated with or without protein phosphatase 2A (PP-2A). Phosphatase treatment converted the upper, gel-shifted, form to the lower band, demonstrating that the gel-shift was a phosphorylation-dependent event. To determine whether phosphorylation of MEKK1 was required for its activity, the ability of immunoprecipitated MEKK1 to phosphorylate its substrate SEK1 was assessed after pretreatment with PP-2A. Immunoprecipitates treated with phosphatase did not phosphorylate SEK1. Thus, phosphorylation of MEKK1 is required for its activation. These results show that UV irradiation induced a rapid phosphorylation of full length MEKK1 followed by its cleavage into fragments. The extent of JNK activation after UV irradiation paralleled the extent of MEKK1 phosphorylation, consistent with the fact that MEKK1 is an upstream regulator of the JNK pathway. Apoptosis, as assessed by morphological changes of the nucleus, started to be detected 8 hours after UV irradiation and was most apparent after 20 hours.

Cleavage of MEKK1 can be Mediated by Different Stress Stimuli.

Several genotoxic stress stimuli were applied to HEK293 cells and their effect on the MEKK1 protein was assessed. UV irradiation, cisplatin, etoposide and mitomycin C induced the loss of full length MEKK1 and the appearance of a lower molecular weight fragment derived from MEKK1. While there was no full length MEKK1 protein remaining after UV and cisplatin treatments, a small amount of upward gel-shifted full length MEKK1 was detected in etoposide and mitomycin C-treated cells. This indicates that chemicals capable of forming DNA adducts, induce the phosphorylation of MEKK1 before its cleavage. These results indicate that the cleavage of MEKK1 may be the activation step leading to apoptosis in a number of stress conditions.

Discussion

An emerging theme for the cellular commitment to apoptosis involves the activation of specific proteases and the regulation of signal transduction pathways, but the integration of these two regulatory processes in the apoptotic response has not been clearly defined. The role of ICE/FLICE proteases being involved in the apoptotic response is unequivocal (Fraser and Evan, 1996). Loss or inhibition of these enzyme activities can inhibit apoptosis (Los et al., 1995; Darmon and Bleackley, 1996). The notion that signal transduction pathways, specifically those involving the c-Jun kinases and p38 kinases, has developed based on correlative biochemical analysis and gene transfection experiments. An inhibitory mutant of SEK1 (c-Jun kinase kinase) was demonstrated to block ceramide-induced apoptosis in different cell types (Verheij et al., 1996). Similarly, it was shown that a dominant negative c-Jun mutant could block apoptosis of serum-deprived neuronal cells (Xia et al., 1995). Activated mutants of p38 and its immediate upstream regulatory kinase MKK3 was shown to enhance an apoptotic response of PC12 cells to serum deprivation (Xia et al., 1995). The ERK pathway has been shown to have a protective response against an apoptotic stimulus in a few cell types (Xia et al., 1995; Gardner and Johnson, 1996). However, discordance for a role of c-Jun kinases and p38 kinases in mediating apoptosis also exists. For example, MEKK1 mediated apoptosis was shown to be independent of c-Jun kinase activation (Lassignal Johnson et al., 1996). A similar separation of c-Jun kinase activation and apoptosis was observed with the TNF receptor (Liu et al., 1996b).

In this example, we show that the JNK pathway is clearly not sufficient to induce the apoptosis mediated by MEKK1. Numerous other examples exist where c-Jun kinase and p38 are activated in response to a stimulus but apoptosis is not observed (Su et al., 1994; Sumimoto et al., 1994; Tsubata et al., 1993). What is however evolving from these studies is that the integration of several different signals, including the regulation of MAP kinase pathways (Xia et al., 1995; Gardner and Johnson, 1996), can contribute to the decision of a cell to commit to apoptosis. Just as with growth and differentiation a series of checkpoints must be overcome before a cell commits itself to death. The needed commitment appears to be activation of the ICE/FLICE protease cascade; activation of c-Jun kinase or p38 pathways may be insufficient by themselves but may enhance or prevent the apoptotic response resulting from an external stimulus such as a genotoxic agent or cytokine.

MEKK1-mediated Apoptosis Requires Both Kinase Activity and Proteolytic Cleavage.

We have shown previously that the kinase activity of MEKK1 is required for its apoptotic activity, because the kinase-inactive (MEKK1 is unable to promote apoptosis (Lassignal Johnson et al., 1996). Here we show that there is a tight integration of kinase and protease activities in the MEKK1-induced apoptotic pathway. Proteases are required for MEKK1-induced apoptosis at at least two levels in the transduction pathway. The first level corresponds to the cleavage of MEKK1 at position 874 in the mouse MEKK1 sequence. When this cleavage is prevented by the p35 baculovirus protein or when a cleavage-resistant MEKK1 mutant is used, apoptosis is strongly impaired. Proteases of the ICE family of proteases are required for this cleavage to occur, since the viral inhibitors CrmA and p35 inhibit the cleavage. It is indeed likely that CPP32 or a CPP32-like enzyme directly cleaves MEKK1 at position 874, because the recognition site for the protease in the mouse MEKK1 is DTVD, a sequence that closely resembles the DEVD recognition site of the CPP32 substrate poly (ADP-ribose) polymerase (Nicholson et al., 1995). The sequence in the rat MEKK1 sequence that corresponds to the murine DTVD cleavage recognition site is DTLD (Xu et al., 1996); indicating that the cleavage site is conserved between the mouse and the rat MEKK1 proteins and further supports its importance in MEKK1 function. ICE-like proteases are also required at a second step that is downstream of the cleavage of MEKK1 because p35 inhibits the apoptosis induced by the kinase domain of MEKK1.

FIG. 8 shows a model defining the involvement of MEKK1 in apoptosis. The 196 kDa MEKK1 protein can be activated by many extracellular inputs including tyrosine kinase encoded growth factor receptors, G protein-coupled receptors (Avdi et al., 1996) and cellular stresses. Activation of MEKK1 correlates with its phosphorylation. It is unclear at present if MEKK1 phosphorylation involves autophosphorylation or additional kinases. Activated MEKK1 independent of its proteolysis is capable of regulating the c-Jun kinase pathway and may also regulate the ERK pathway. Both of these pathways can stimulate anti-apoptotic responses. Stimulation of the JNK pathway can lead to NFKB activation which is a strong inibitor of apoptosis (Baeuerle and Baltimore, 1996) and activation of the ERK pathway has been shown to protect cells from apoptosis (Xia et al., 1995; Gardner and Johnson, 1996). With an appropriate protease activation MEKK1 is cleaved to generate a 91 kDa activated kinase domain that has substrates that contribute to driving the cell to apoptosis. Downstream of these phosphorylation events are additional protease substrates that are predicted to be either phosphoproteins or proteins whose activity is regulated by phosphoproteins and which are involved in regulating apoptosis. Bcl-2, for example, would be such a phosphoprotein candidate (Gajewski and Thompson, 1996).

Proteolysis of MEKK1 generates an Activated Fragment with Altered Cellular Distribution.

We have found that the endogenous MEKK1 in resting cells is localized in a post-Golgi vesicular compartment. The punctate cytoplasmic staining of MEKK1 can be seen in non-transfected cells. Upon appropriate cellular stimulation by a growth factor such as EGF MEKK1 is translocated to the plasma membrane. When MEKK1 is overexpressed it is activated and becomes proteolyzed. When MEKK1 is proteolyzed the catalytic domain behaves as a soluble cytoplasmic protein that is no longer sequestered on vesicle-like structures or the plasma membrane. Cleavage of MEKK1 may also change the specificity and activity of the kinase. In vitro kinase assays have indeed revealed that the kinase activity of the cleaved MEKK1 towards SEK1 is increased compared to the full length MEKK1. Thus, the 91 kDa kinase fragment of MEKK1 has a different subcellular distribution from the 196 kDa holo-MEKK1 which may allow it to phosphorylate a different set of substrates.

Genotoxic stress: A Balance Between Rescue and Suicide using MEKK1 as a Switch.

Our results show that DNA damaging chemicals such as cisplatin, etoposide and mitomycin C in addition to UV irradiation induce a phosphorylation correlated with activation of MEKK1. The time course for UV irradiation-induced c-Jun kinase activation closely paralleled that for MEKK1 phosphorylation, consistent with MEKK1 being an upstream regulator of this pathway. Thus, UV irradiation induces a rapid phosphorylation and activation of MEKK1 and c-Jun kinase. The rapid c-Jun kinase response could actually contribute to a protective response against cell death. This has been proposed for the action of CD40 in protecting B cells from antigen crosslinking-induced apoptosis (Sumimoto et al., 1994; Tsubata et al., 1993) and methyl methane sulfonate-induced 3T3 cell apoptosis (Liu et al., 1996a). The activation of NFKB in response to stresses including UV irradiation and genotoxic chemicals would also be a protective response (Baeuerle and Baltimore, 1996); MEKK1 has been shown to be involved in the activation of NFKB (Hirano et al., 1996).

If the stress challenge to the cell is too great a protease cascade is activated involving the ICE/FLICE enzymes (Fraser and Evan, 1996). Our data indicate that one substrate for CPP32-like proteases is MEKK1. The time course of MEKK1 proteolysis is slower than its activation; cleavage of MEKK1 releases the 91 kDa kinase domain with new subcellular localization and the ability to activate effectors of apoptosis.

These findings suggest MEKK1 can function as a switch point, regulated by a proteolytic event controlled by ICE/FLICE proteases, that determines cell fate in response to a stress stimulus. Before cleavage MEKK1 induces rescue mechanisms and after cleavage MEKK1 triggers apoptosis. The cleavage of MEKK1 may thus occur when the cell has failed to successfully repair itself. The cleaved MEKK1 then triggers apoptosis which leads to the elimination of the cell.

Conclusion

Our studies define MEKK1 as a protease substrate that when activated and cleaved stimulates an apoptotic response. The proteolytic cleavage of MEKK1 defines the mechanism to generate a protein kinase whose activity is sufficient to induce apoptosis. In the context of cancer therapy, our finding that the activation and cleavage of MEKK1 occurs in response to genotoxic agents is particularly important. We have found that expression of MEKK1 is capable of killing by apoptosis cells that have both p53 alleles mutated. Hence, the activation and cleavage of MEKK1 is an apoptotic pathway that does not require a functional p53 and stimulation of these events could enhance the killing of many different tumors. Manipulating the activation of MEKK1 and its cleavage by proteases, with the use of drugs for example, could increase the killing of tumor cells to genotoxic agents. Consistent with this hypothesis is our finding that low level expression of MEKK1 potentiated the apoptotic response to low doses of UV irradiation and cisplatin.

Citations for Publications Referred to in this Example:

An, B. and Dou, Q. P. (1996). Cleavage of retinoblastoma protein during apoptosis: an interleukin 1β-converting enzyme-like protease as candidate. Cancer Res. 56, 438-442.

Avdi, N. J., Winston, B. W., Russel, M., Young, S. K., Johnson, G. L., and Worthen, G. S. (1996). Activation of MEKK by Formyl-methionyl-leucyl-phenylalanine in Human Neutrophils. Mapping pathways for mitogen-activated protein kinase activation. J. Biol. Chem. 271, 33598-33606.

Baeuerle, P. A. and Baltimore, D. (1996). NF-(kappa)B: ten years after. Cell 87, 13-20.

Beidler, D. R., Tewari, M., Friesen, P. D., Poirier, G., and Dixit, V. M. (1996). The baculovirus p35 protein inhibits Fas- and tumor necrosis factor-induced apoptosis. J. Biol. Chem. 270, 16526-16528.

Blank, J. L., Gerwins, P., Elliott, E. M., Sather, S., and Johnson, G. L. (1996). Molecular cloning of mitogen-activated protein/ERK kinase kinases (MEKK) 2 and 3. Regulation of sequential phosphorylation pathways involving mitogen-activated protein kinase and c-Jun kinase. J. Biol. Chem. 271, 5361-5368.

Canman, C. E. and Kastan, M. B. (1996). Three paths to stress relief. Nature 384, 213-214.

Cartier, J. L., Hershberger, P. A., and Friesen, P. D. (1994). Suppression of apoptosis in insect cells stably transfected with baculovirusp35: dominant interference by N-terminal sequences p351-76. J. Virol. 68, 7728-7737.

Casciola-Rosen, L. A., Miller, D. K., Anhalt, G. J., and Rosen, A. (1994). Specific cleavage of the 70-kDa protein component of the U1 small nuclear ribonucleoprotein is a characteristic biochemical feature of apoptotic cell death. J. Biol. Chem. 269, 30757-30760.

Casciola-Rosen, L. A., Anhalt, G. J., and Rosen, A. (1995). DNA-dependent protein kinase is one of a subset of antoantigen specifically cleaved early during apoptosis. J. Exp. Med. 182, 1625-1634.

Clem, R. J., Hardwick, J. M., and Miller, L. K. (1996). Anti-apoptotic genes of baculoviruses. Death Differ. 3, 9-16.

Cryns, V. L., Bergeron, L., Zhu, H., Li, H., and Yuan, J. (1996). Specific cleavage of α-fodrin during Fas- and tumor necrosis factor-induced apoptosis is mediated by an interleukin-1β-converting enzyme/Ced-3 protease distinct from the poly(ADP-ribose) polymerase protease. J. Biol. Chem. 271, 31277-31282.

Darmon, A. J. and Bleackley, R. C. (1996). An Interleukin-1 Converting Enzyme-like Protease Is a Key Component of Fas-mediated Apoptosis. J. Biol. Chem. 271, 21699-21702.

Dérijard, B., Hibi, M., Wu, I., Barrett, T., Su, B., Deng, T., Karin, M., and Davis, R. J. (1994). JNK-1: a protein kinase stimulated by UV light and Ha-Ras that binds and phosphorylates the c-jun activation domain. Cell 76, 1025-1037.

Emoto, Y., Manome, Y., Meinhardt, G., Kisaki, H., Kharbanda, S., Robertson, M., Ghayur, T., Wong, W. W., Kamen, R., Weichselbaum, R., and Kufe, D. (1995). Proteolytic activation of protein kinase C 8 by an ICE-like protease in apoptotic cells. EMBO J. 14, 6148-6156.

Esolen, L. M., Park, S. W., Hardwick, J. M., and Griffin, D. E. (1995). Apoptosis as a cause of death in measles virus-infected cells. J. Virol. 69, 3955-3958.

Fraser, A. and Evan, G. (1996). A license to kill. Cell 85, 781-784.

Gajewski, T. F. and Thompson, C. B. (1996). Apoptosis meets signal transduction: elimination of a BAD influence. Cell 87, 589-592.

Gardner, A. M. and Johnson, G. L. (1996). Fibroblast growth factor-2 suppression of tumor necrosis factor α-mediated apoptosis requires ras and the activation of mitogen-activated protein kinase. J. Biol. Chem. 271, 14560-14566.

Gupta, S., Barrett, T., Whitmarsh, A. J., Cavanagh, J., Sluss, H. K., Derijard, B., and Davis, R. J. (1996). Selective interaction of JNK protein kinase isoforms with transcription factors. EMBO J. 15, 2760-2770.

Hibi, M., Lin, A., Smeal, T., Minden, A., and Karin, M. (1993). Identification of an oncoprotein- and UV-responsive protein kinase that binds and potentiates the c-Jun activation domain. Genes Develop. 7, 2135-2148.

Hinshaw, V. S., Olsen, C. W., Dybdahl-Sissoko, N., and Evans, D. (1994). Apoptosis: a mechanism of cell killing by influenza A and B viruses. J. Virol. 68, 3667-3673.

Hirano, M., Osada, S.-i., Aoki, T., Hirai, S. -i., Hosaka, M., Inoue, J. -i., and Ohno, S. (1996). MEK kinase is involved in tumor necrosis factor α-induced NF-kB activation and degradation of IkB-α. J. Biol. Chem. 271, 13234-13238.

Howard, A. D., Kostura, M. J., Thomberry, N., Ding, G. J. -F., Limjuco, G., Weidner, J., Salley, J. P., Hogquist, K. A., Chaplin, D. D., Mumford, R. A., Schmidt, J. A., and Tocci, M. J. (1991). IL-1-converting enzyme requires aspartic acid residues for processing of the IL-1β precursor at two distinct sites and does not cleave 31-kDa IL-1α. J. Immunol. 147, 2964-2969.

Juo, P., Kuo, C. J., Reynolds, S. E., Konz, R. F., Raingeaud, J., Davis, R. J., Biemann, H. -P., and Blenis, J. (1997). Fas activation of the p38 mitogen-activated protein kinase signalling pathway requires ICE/CED-3 family proteases. Mol. Cell. Biol. 17, 24-35.

Kallunki, T., Su, B., Tsigelny, I., Sluss, H. K., Dérijard, B., Moore, G., Davis, R. J., and Karin, M. (1994). JNK2 contains a specificity-determin;ig region responsible for efficient c-Jun binding and phosphorylation. Genes Develop. 8, 2996-3007.

Kagi, D., Vignaux, F., Ledermann, B., Büirki, K., Depraetere, V., Nagata, S., Hengartner, H., and Golstein, P. (1994). Fas and perforin pathways as major mechanisms of T cell-mediated cytotoxicity. Science 265, 528-530.

Lange-Carter, C. A., Pleiman, C. M., Gardner, A. M., Blumer, K. J., and Johnson, G. L. (1993). A divergence in the MAP kinase regulatory network defined by MEK kinase and Raf. Science 260, 315-319.

Lange-Carter, C. A. and Johnson, G. L. (1994). Ras-dependent growth factor regulation of MEK kinase in PC12 cells. Science 265, 1458-1461.

Lassignal Johnson, N., Gardner, A. M., Diener, K. M., Lange-Carter, C. A., Gleavy, J., Jarpe, M. B., Minden, A., Karin, M., Zon, L. I., and Johnson, G. L. (1996). Signal transduction pathways regulated by mitogen-activated/extracellular response kinase kinase kinase induce cell death. J. Biol. Chem. 271, 3229-3237.

Lazebnik, Y. A., Kaufmann, S. H., Desnoyers, S., Poirier, G. G., and Earnshaw, W. C. (1994). Cleavage of poly(ADP-ribose) plymerase by a proteinase with properties like ICE. Nature 371, 346-347.

Lazebnik, Y. A., Takahashi, A., Moir, R. D., Goldman, R. D., Poirier, G. G., Kaufmann, S. H., and Earnshaw, W. C. (1995). Studies of the lamin proteinase reveal multiple parallel biochemical pathways during apoptotic execution. Proc. Natn. Acad. Sci. U. S. A. 92, 9042-9046.

Liu, Z. -G., Baskaran, R., Lea-Chou, E. T., Wood, L. D., Chen, Y., Karin, M., and Wang, J. Y. J. (1996a). Three distinct signalling response by murine fibroblasts to genotoxic stress. Nature 384, 273-276.

Liu, Z. -G., Hsu, H., Goeddel, D. V., and Karin, M. (1996b). Dissection of TNF receptor 1 effector functions: JNK activation is not linked to apoptosis while NF-kB activation prevents cell death. Cell 87, 565-576.

Los, M., Van de Craen, M., Penning, L. C., Schenk, H., Westendorp, M., Baeuerle, P. A., Dröge, W., Krammer, P. H., Fiers, W., and Schulze-Osthoff, K. (1995). Requirement of an ICE/CED-3 protease for Fas/APO-1-mediated apoptosis. Nature 375, 81-83.

Lowin, B., Hahne, M., Mattmann, C., and Tschopp, J. (1994). Cytolytic T-cell cytotoxicity is mediated through perforin and Fas lytic pathways. Nature 370, 650-652.

Na, S., Chuang, T. -H., Cunningham, A., Turi, T. G., Hanke, J. H., Bokoch, G. M., and Danley, D. E. (1996). D4-GDI, a Substrate of CPP32, Is Proteolyzed during Fas-induced Apoptosis. J. Biol. Chem. 271, 11209-11213.

Nicholson, D. W., Ali, A., Thornberry, N. A., Vaillancourt, J. P., Ding, C. K., Gallan, M., Gareau, Y., Griffin, P. R., Labelle, M., Lazebnik, Y. A., Munday, N. A., Raju, S. M., Smulson, M. E., Yamin, T. -T., Yu, V. L., and Miller, D. K. (1995). Identification and inhibition of the ICE/CED-3 protease necessary for mammalian apoptosis. Nature 376, 37-43.

Nicoletti, I., Miglioratti, G., Pagliacci, M. C., Grignani, F., and Riccardi, C. (1991). A rapid and simple method for measuring thymocyte apoptosis by propidium iodide staining and flow cytometry. J. Immunol. Methods 139, 271-279.

Orth, K., O'Rourke, K., Salvesen, G. S., and Dixit, V. M. (1996). Molecular Ordering of Apoptotic Mammalian CED-3/ICE-like Proteases. J. Biol. Chem. 271, 20977-20980.

Park, D. S., Stefanis, L., Yan, C. Y. I., Farinelli, S. E., and Greene, L. A. (1996). Ordering the Cell Death Pathway. Differential effects of Bcl2, an interleukin-1β-converting enzyme family protease inhibitor, and other survival agents of JNK activation in serum/nerve growth factor-deprived PC12 cells. J. Biol. Chem. 271, 21898-21905.

Pickup, D. J., Ink, B. S., Hu, W., Ray, C. A., and Joklik, W. K. (1986). Hemorrhage in lesions caused by cowpox virus is induced by a viral protein that is related to plasma protein inhibitors of serine proteases. Proc. Natn. Acad. Sci. U. S. A. 83, 7698-7702.

Steller, H. (1995). Mechanisms and genes of cellular suicide. Science 267, 1445-1449.

Su, B., Jacinto, E., Hibi, M., Kallunki, T., Karin, M., and Ben-Neriah, Y. (1994). JNK is involved in signal integration during costimulation of T lymphocytes. Cell 77, 727-736.

Sumimoto, S. -I., Heike, T., Kanazashi, S. -I., Shintaku, N., Jung, E. -Y., Hata, D., Katamura, K., and Mayumi, M. (1994). Involvement of LFA-1/intracellular adhesion molecule-1-dependent cell adhesion in CD40-mediated inhibition of human B lymphoma cell death induced by surface IgM crosslinking. J. Immunol. 153, 2488-2496.

Terai, C., Kombluth, R. S., Pauza, C. D., Richman, D. D., and Carson, D. A. (1991). Apoptosis as a mechanis of cell death in cultured T lymphoblasts acutely infected with HIV-1. J. Clin. Invest. 87, 1710-1715.

Tsubata, T., Wu, J., and Honjo, T. (1993). B-cell apoptosis induced by antigen receptor crosslinking is blocked by a T-cell signal through CD40. Nature 364, 645-648.

Tyler, K. L., Squier, M. K. T., Rodgers, S. E., Schneider, B. E., Oberhaus, S. M., Grdina, T. A., Cohen, J. J., and Dermody, T. S. (1995). Differences in the capacity of reovirus strains to induce apoptosis are determined by the viral attachment protein σ1. J. Virol. 69, 6972-6979.

Vandenabeele, P., Declercg, W., Beyaert, R., and Fiers, W. (1995). Two tumour necrosis factor receptors: structure and function. Trends Cell Biol. 5, 392-399.

Verheij, M., Bose, R., Lin, X. H., Yao, B., Jarvis, W. D., Grant, S., Birrer, M. J., Szabo, E., Zon, L. I., Kyriakis, J. M., Haimovitz-Friedman, A., Fuks, Z., and Kolesnick, R. N. (1996). Requirement for ceramide-initiated SAPK/JNK signalling in stress-induced apoptosis. Nature 380, 75-79.

Wang, X., Zelenski, N. G., Yang, J., Sakai, J., Brown, M. S., and Goldstein, J. L. (1996). Cleavage of sterol regulatory element binding protein (SREBPs) by CPP32 during apoptosis. EMBO J. 15, 1012-1020.

Widmann, C., Dolci, W., and Thorens, B. (1995). Agonist-induced internalization and recycling of the glucagon-like peptide-1 receptor in transfected fibroblasts and in insulinomas. Biochem. J. 310, 203-214.

Williams, G. T. and Smith, C. A. (1993). Molecular regulation of apoptosis: genetic controls on cell death. Cell 74, 777-779.

Xia, Z., Dickens, M., Raingeaud, J., Davis, R. J., and Greenberg, M. E. (1995). Opposing effects of ERK and JNK-p38 MAP kinases on apoptosis. Science 270, 1326-1331.

Xu, S., Robbins, D. J., Christerson, L. B., English, J. M., Vanderbilt, C. A., and Cobb, M. H. (1996). Cloning of rat MEK kinase 1 cDNA reveals an endogenous membrane-associated 195-kDa protein with a large regulatory domain. Proc. Natn. Acad. Sci. U. S. A. 93, 5291-5295.

The foregoing description of the invention has been presented for purposes of illustration and description. Further, the description is not intended to limit the invention to the form disclosed herein. Consequently, variations and modifications commensurate with the above teachings, and the skill or knowledge in the relevant art are within the scope of the present invention. The preferred embodiment described herein above is further intended to explain the best mode known of practicing the invention and to enable others skilled in the art to utilize the invention in various embodiments and with various modifications required by their particular applications or uses of the invention. It is intended that the appended claims be construed to include alternate embodiments to the extent permitted by the prior art.

We claim:

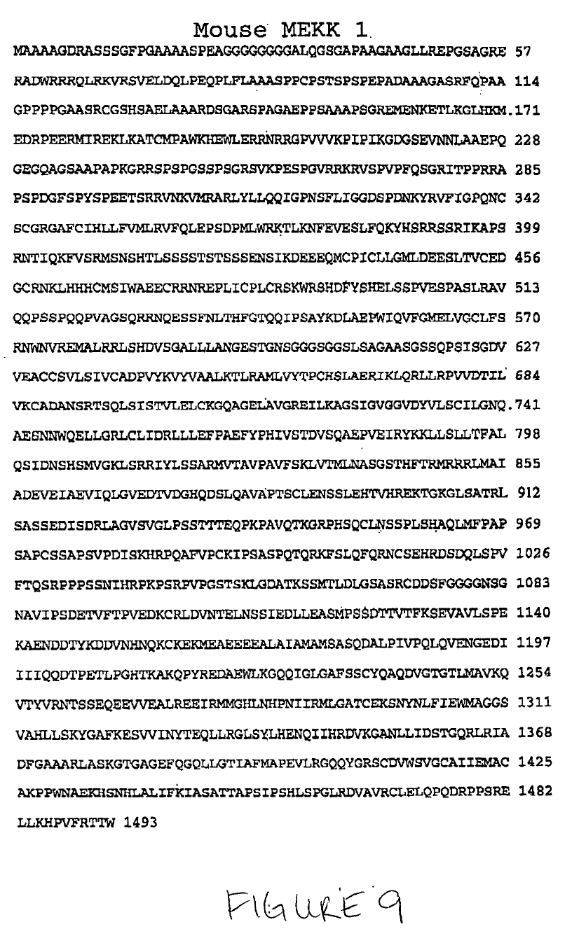

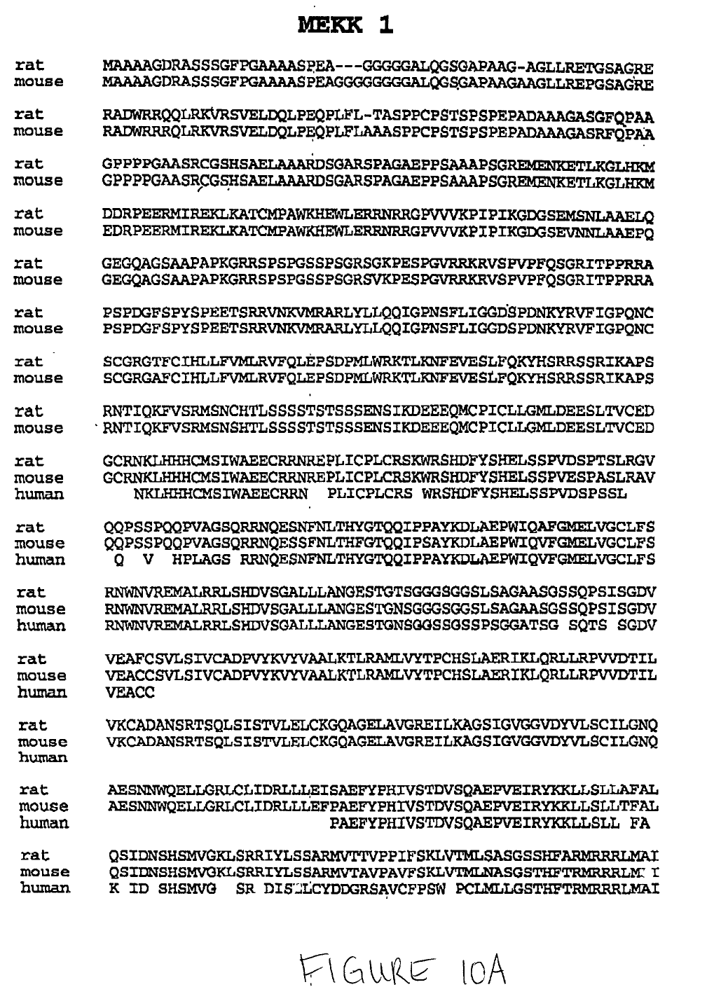

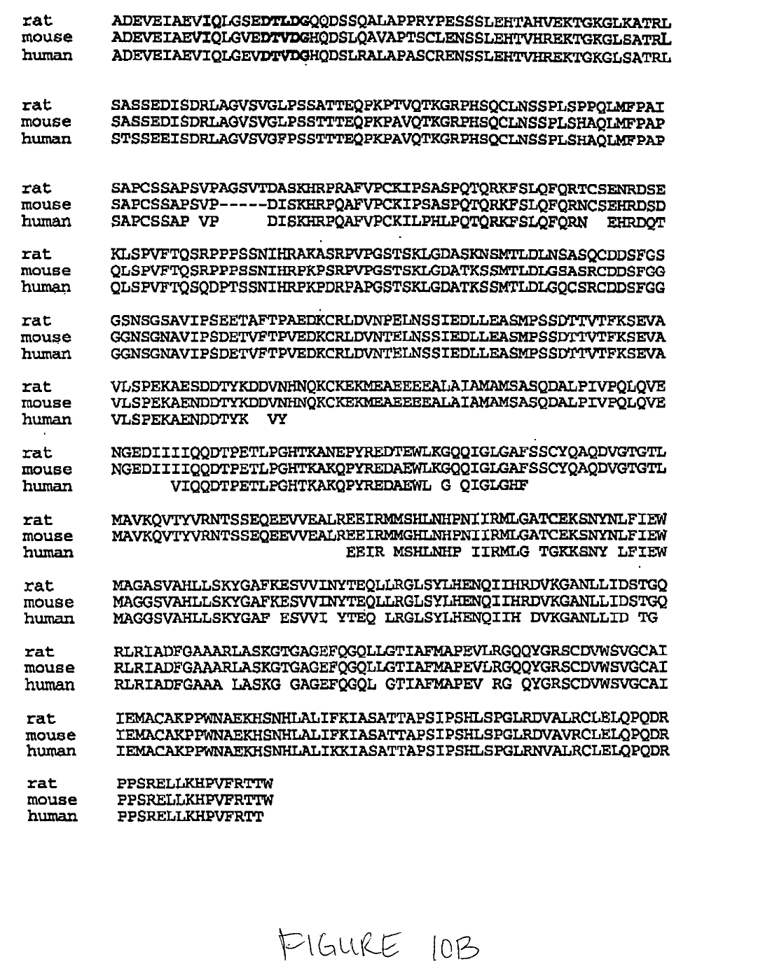

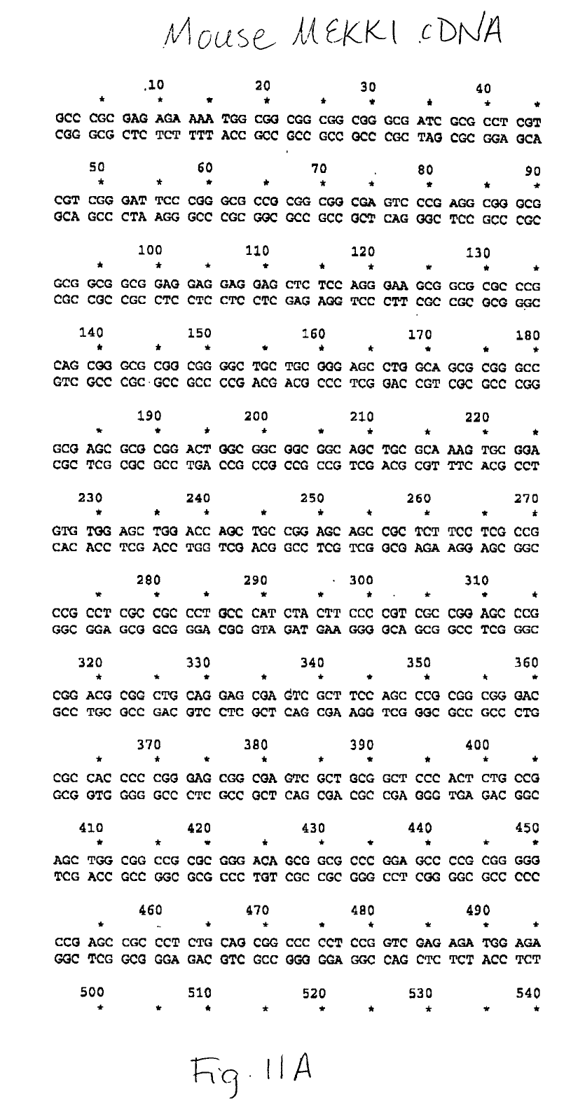

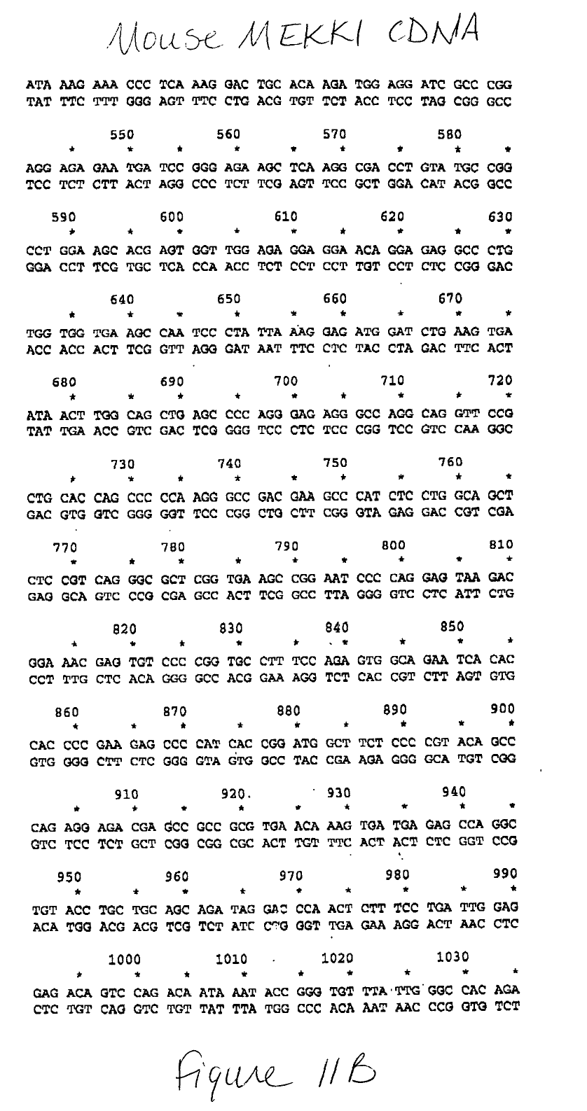

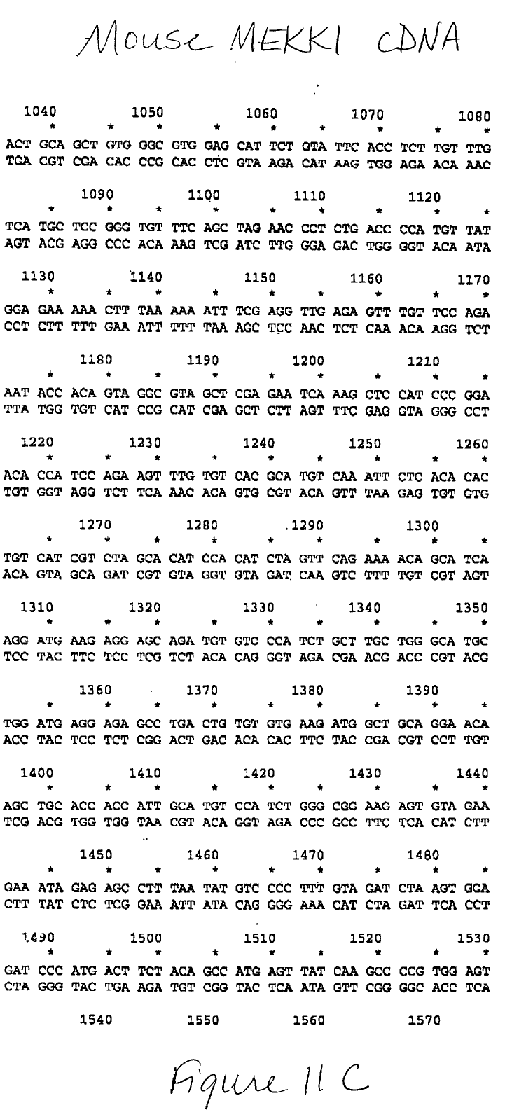

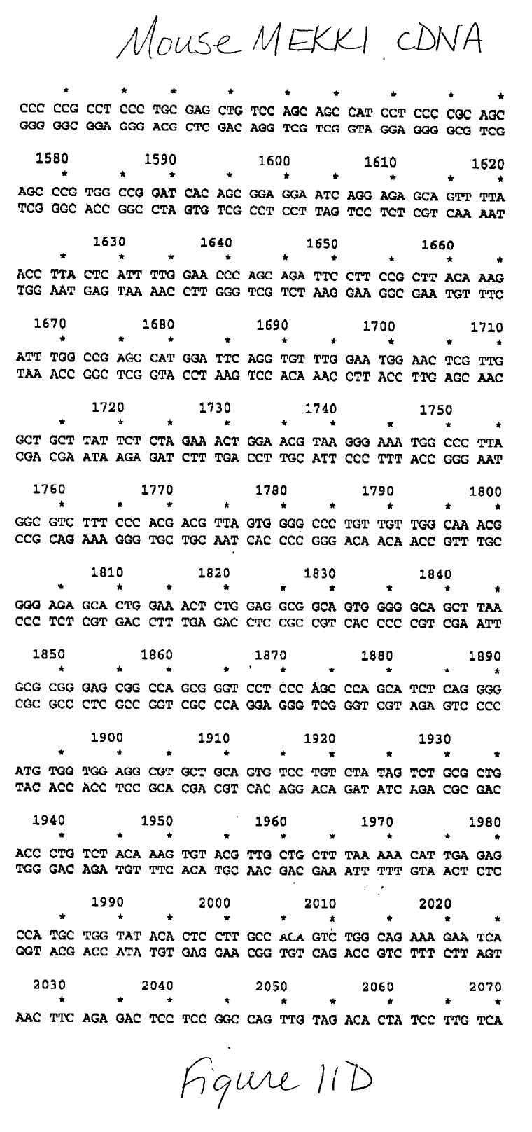

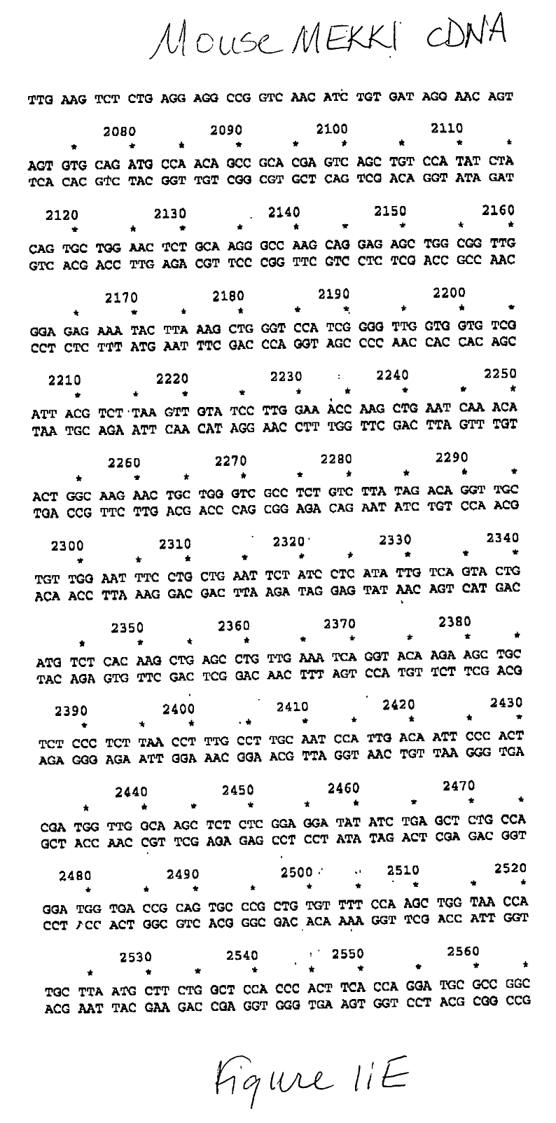

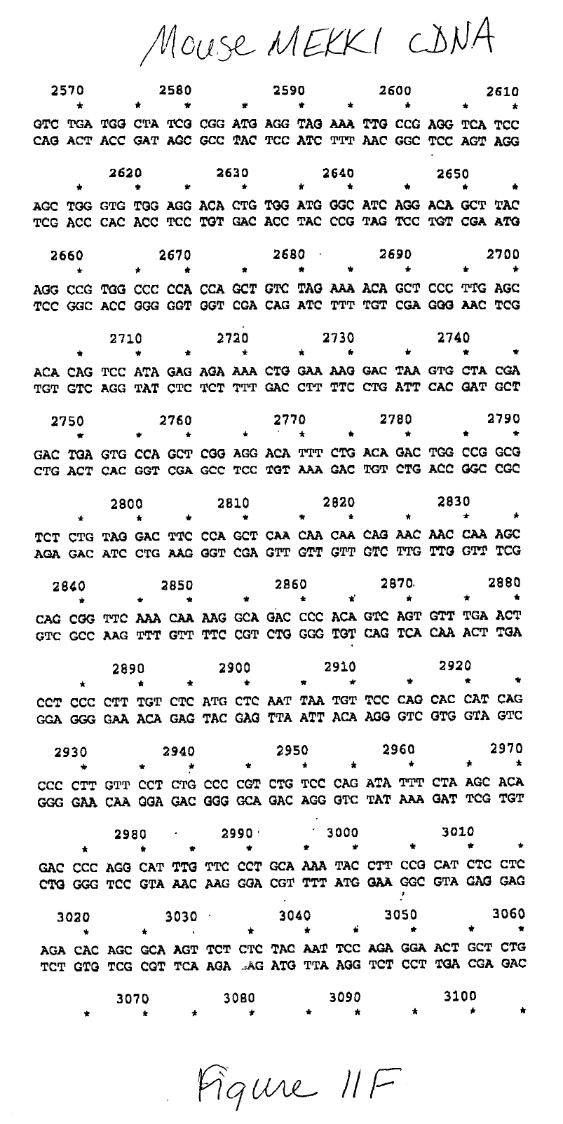

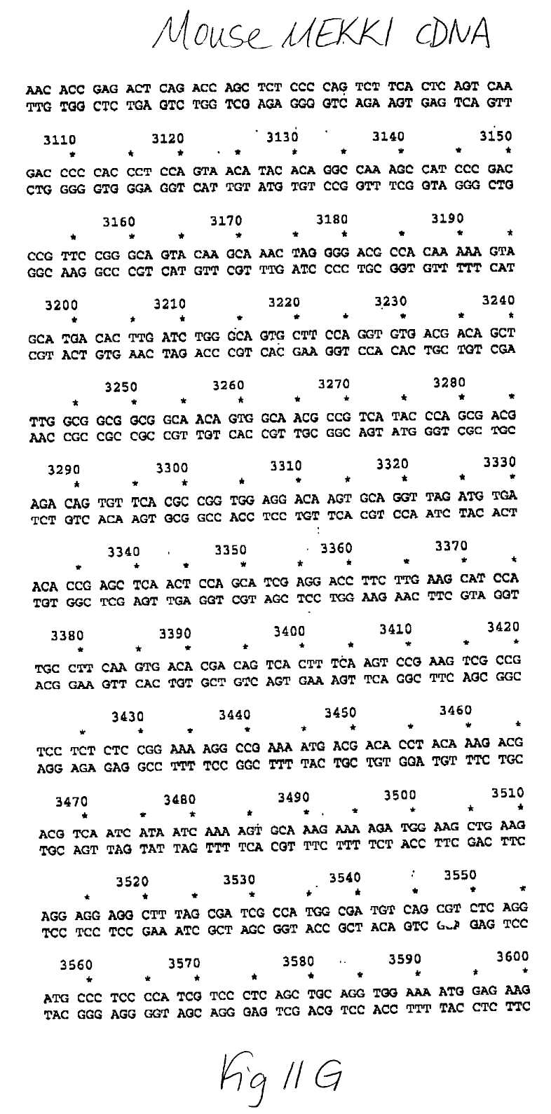

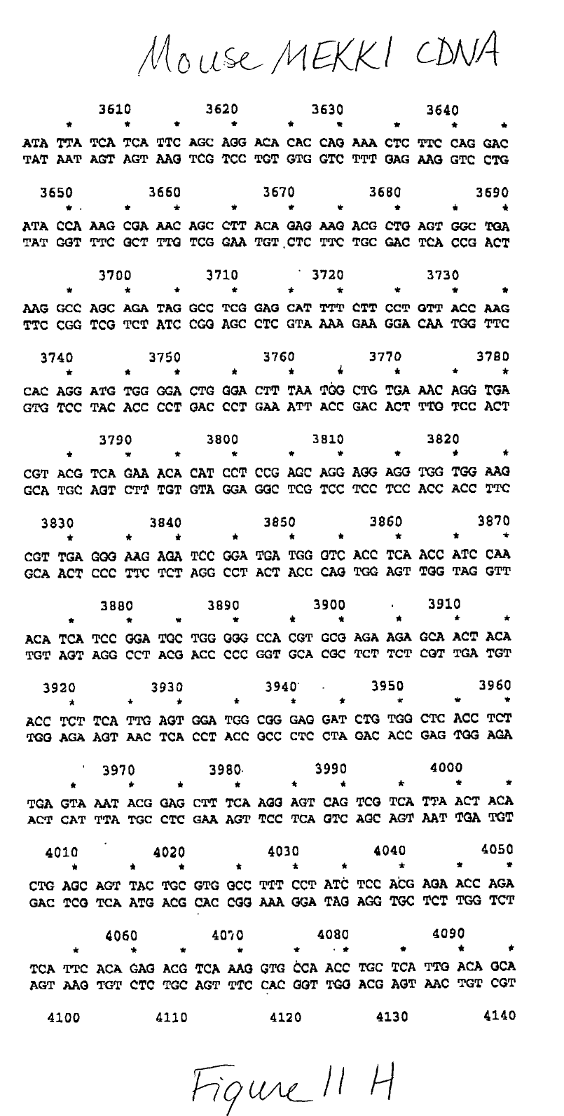

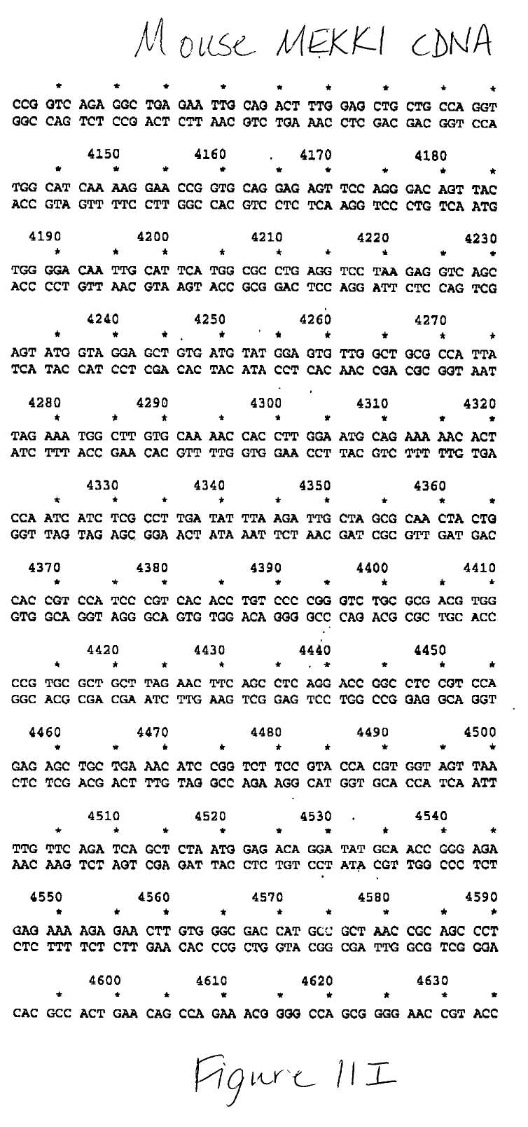

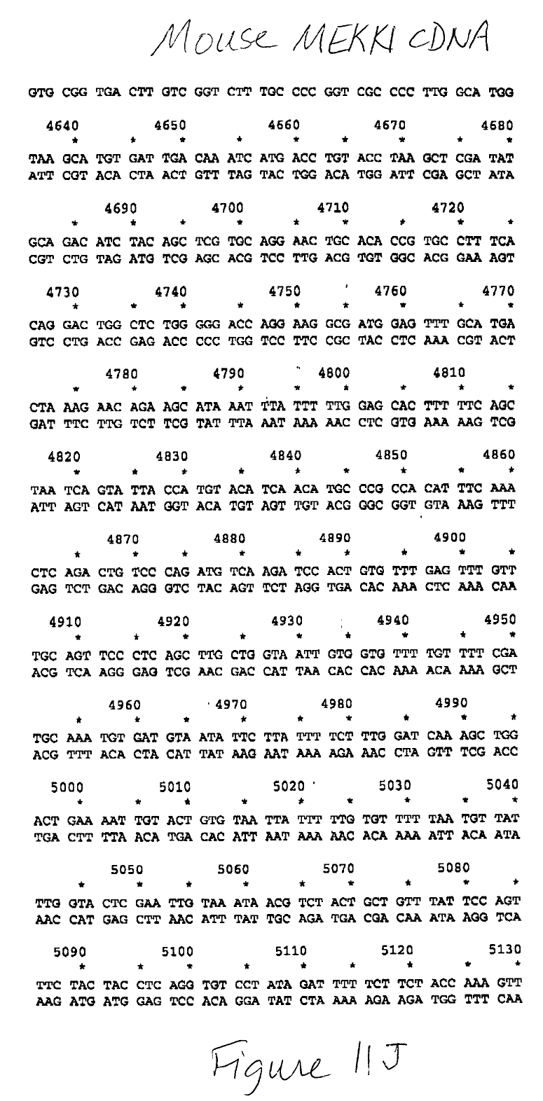

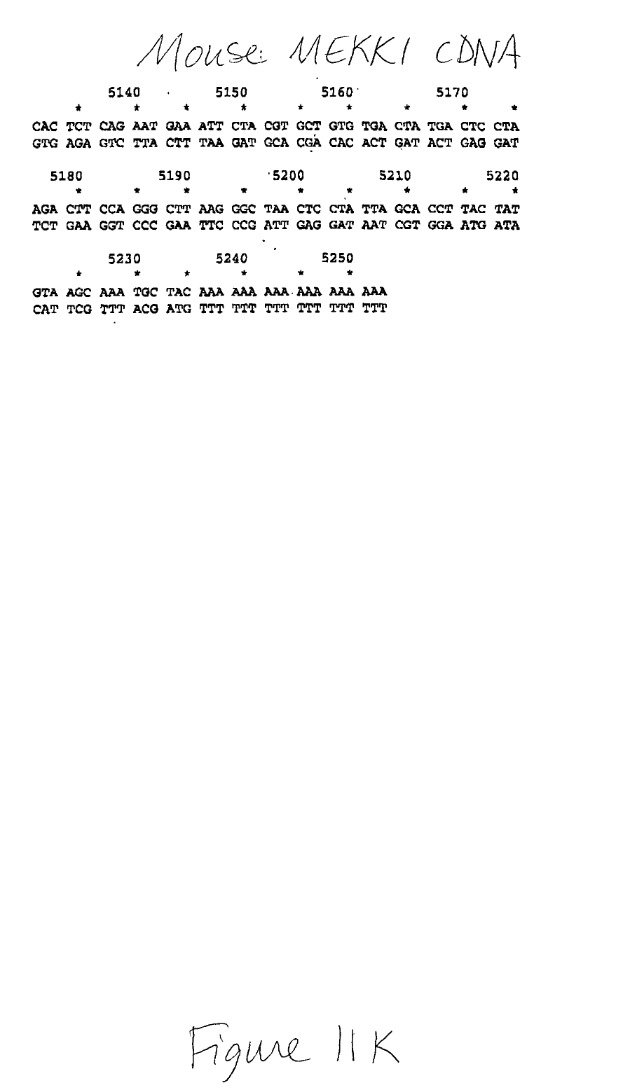

1. An isolated active fragment of an MEKK1 protein consisting of an amino acid sequence having at least 75% homology to an amino acid sequence consisting of about amino acids 875-1493 of FIG. 9, wherein said active fragment mediates apoptosis.

2. The active fragment of claim 1, which consists of an amino acid sequence having at least 85% homology to an amino acid sequence consisting of about amino acids 875-1493 of FIG. 9.

3. The active fragment of claim 1, which consists of an amino acid sequence having at least 95% homology to an amino acid sequence consisting of about amino acids 875-1493 of FIG. 9.

4. The active fragment of claim 1, which is a mouse MEKK1 active fragment.

5. The active fragment of claim 1, which is a human MEKK1 active fragment.

6. The active fragment of claim 1, which is a rat MEKK1 active fragment.

7. The active fragment of claim 1, which consists of about amino acids 875-1493 of FIG. 9.

8. The active fragment of claim 1, which consists of amino acids 875-1493 of FIG. 9.

9. An isolated protease-resistant MEKK1 protein comprising an amino acid sequence having at least 75% homology to the amino acid sequence of FIG. 9, wherein at least one amino acid equivalent to amino acids 871-874 of FIG. 9 is substituted such that the MEKK1 protein is resistant to proteolysis by a caspase after amino acid 874.

10. The MEKK1 protein of claim 9, wherein at least one amino acid equivalent to amino acids 871-874 of FIG. 9 is substituted with an alanine residue.

11. The MEKK1 protein of claim 9, wherein each amino acid equivalent to amino acids 871-874 of FIG. 9 is substituted with an alanine residue.

12. The MEKK1 protein of claim 9, which has at least 85% homology to the amino acid sequence of FIG. 9.

13. The MEKK1 protein of claim 9, which has at least 95% homology to the amino acid sequence of FIG. 9.

14. The MEKK1 protein of claim 9, which is a mouse MEKK1 protein.

15. The MEKK1 protein of claim 9, which is a human MEKK1 protein.

16. The MEKK1 protein of claim 9, which is a rat MEKK1 protein.

17. An isolated nucleic acid molecule consisting of a nucleotide sequence having at least 75% homology to a nucleotide sequence consisting of about nucleotides 645-2501 of SEQ ID NO: 1, wherein said nucleic acid molecule encodes an active fragment of MEKK1 that mediates apoptosis.

18. The nucleic acid molecule of claim 17, which consists of a nucleotide sequence having at least 85% homology to a nucleotide sequence consisting of about nucleotides 645-2501 of SEQ ID NO: 1.

19. The nucleic acid molecule of claim 17, which consists of a nucleotide sequence having at least 95% homology to a nucleotide sequence consisting of about nucleotides 645-2501 of SEQ ID NO:1.

20. The nucleic acid molecule of claim 17, which encodes an active fragment of a mouse MEKK1.

21. The nucleic acid molecule of claim 17, which encodes an active fragment of a human MEKK1.

22. The nucleic acid molecule of claim 17, which encodes an active fragment of a rat MEKK1.

23. The nucleic acid molecule of claim 17, which consists of about nucleotides 645-2501 of SEQ ID NO: 1, or a nucleotide sequence that, due to the degeneracy of the genetic code, encodes the same amino acid sequence as about nucleotides 645-2501 of SEQ ID NO: 1.

24. The nucleic acid molecule of claim 17, which consists of nucleotides 645-2501 of SEQ ID NO: 1, or a nucleotide sequence that, due to the degeneracy of the genetic code, encodes the same amino acid sequence as nucleotides 645-2501 of SEQ ID NO: 1.

25. An isolated nucleic acid molecule encoding a protease-resistant MEKK1 protein, wherein the protease resistant MEKK1 protein comprises an amino acid sequence having at least 75% homology to the amino acid sequence of FIG. 9 and at least one codon of the nucleic acid molecule encoding an amino acid equivalent to at least one of amino acids 871-874 of FIG. 9 is mutated such the encoded MEKK1 protein is resistant to proteolysis by a caspase after an amino acid equivalent to amino acid 874 of FIG. 9.

26. The nucleic acid molecule of claim 25, wherein the MEKK1 protein comprises an amino acid sequence having at least 85% homology to the amino acid sequence of FIG. 9.

27. The nucleic acid molecule of claim 25, wherein the MEKK1 protein comprises an amino acid sequence having at least 95% homology to the amino acid sequence of FIG. 9.

28. The nucleic acid molecule of claim 25, which encodes a protease-resistant mouse MEKK1 protein.

29. The nucleic acid molecule of claim 25, which encodes a protease-resistant human MEKK1 protein.

30. The nucleic acid molecule of claim 25, which encodes a protease-resistant rat MEKK1 protein.

31. An expression vector comprising the nucleic acid molecule of claim 17.

32. An expression vector comprising the nucleic acid molecule of claim 25.

33. A host cell containing the expression vector of claim 31.

34. A host cell containing the expression vector of claim 32.

35. A method of stimulating apoptosis in a cell comprising introducing into the cell an expression vector encoding the MEKK1 active fragment of claim 1 such that MEKK1 active fragment is produced in the cell and apoptosis is stimulated.

36. A method of inhibiting apoptosis in a cell comprising introducing into the cell an expression vector encoding the protease-resistant MEKK1 protein of claim 9 such that protease-resistant MEKK1 protein is produced in the cell and apoptosis is inhibited.

37. A method of generating an MEKK1 active fragment in vitro, comprising:
contacting an MEKK1 protein in vitro with a caspase protease under proteolysis conditions; and
allowing the caspase protease to cleave the MEKK1 protein such that an MEKK1 active fragment is generated.

38. A method of identifying a compound that modulates the apoptotic activity of an MEKK1 active fragment, comprising:
providing an indicator cell that comprises the MEKK1 active fragment of claim 1;
contacting the indicator cell with a test compound; and
determining the effect of the test compound on the apoptotic activity of the MEKK1 active fragment in the indicator cell to thereby identify a compound that modulates the apoptotic activity of the MEKK1 active fragment.

39. A method of identifying a compound that modulates the proteolytic cleavage of an MEKK1 protein by a caspase protease, comprising:
providing a reaction mixture that comprises an MEKK1 protein and a caspase protease;
contacting the reaction mixture with a test compound; and
determining the effect of the test compound on proteolytic cleavage of the MEKK1 protein by the caspase protease to thereby identify a compound that modulates the proteolytic cleavage of an MEKK1 protein by a caspase protease.


SEQUENCE LISTING (13 sequences)

SEQ ID NO: 1 - DNA, 3260 bp, Homo sapiens
  CDS (486)..(2501)
tacactccttgccacagtctggcagaaagaatcaaacttcagagactcctccggccagttgtagacactatccttgtcaagtgtgcagatccaacagccgcacgagtcagctgtccatatctacagtgctggaactctgcaagggccaagcaggagagctggcggttgggagagaaatacttaaagctgggtccatcggggttggtggtgtcgattacgtcttaagttgtatccttggaaaccaagctgaatcaaacaactggcaagaactgctgggtcgcctctgtcttatagacaggttgctgttggaatttcctgctgaattctatcctcatattgtcagtactgatgtctcacaagctgagcctgttgaaatcaggtacaagaagctgctctccctcttaacctttgccttgcaatccattgacaattcccactcgatggttggcaagctctctcggaggatatatctgagctctgccaggatggtgaccgcagtgcccgctgtgttttccaagctggtaaccatgcttaatgcttctggctccacccacttcaccaggatgcgccggcgtctgatggctatcgcggatgaggtagaaattgccgaggtcatccagctgggtgtggaggacactgtggatgggcatcaggacagcttacaggccgtggcccccaccagctgtctagaaaacagctcccttgagcacacagtccatagagagaaaactggaaaaggactaagtgctacgagactgagtgccagctcggaggacatttctgacagactggccggcgtctctgtaggacttcccagctcaacaacaacagaacaaccaaagccagcggttcaaacaaaaggcagaccccacagtcagtgtttgaactcctcccctttgtctcatgctcaattaatgttcccagcaccatcagccccttgttcctctgccccgtctgtcccagatatttctaagcacagaccccaggcatttgttccctgcaaaataccttccgcatctcctcagacacagcgcaagttctctctacaattccagaggaactgctctgaacaccgagactcagaccagctctccccagtcttcactcagtcaagacccccaccctccagtaacatacacaggccaaagccatcccgacccgttccgggcagtacaagcaaactaggggacgccacaaaaagtagcatgacacttgatctgggcagtgcttccaggtgtgacgacagctttggcggcggcggcaacagtggcaacgccgtcatacccagcgacgagacagtgttcacgccggtggaggacaagtgcaggttagatgtgaacaccgagctcaactccagcatcgaggaccttcttgaagcatccatgccttcaagtgacacgacagtcactttcaagtccgaagtcgccgtcctctctccggaaaaggccgaaaatgacgacacctacaaagacgacgtcaatcataatcaaaagtgcaaagaaaagatggaagctgaagaggaggaggctttagcgatcgccatggcgatgtcagcgtctcaggatgccctccccatcgtccctcagctgcaggtggaaaatggagaagatattatcatcattcagcaggacacaccagaaactcttccaggacataccaaagcgaaacagccttacagagaagacgctgagtggctgaaaggccagcagataggcctcggagcattttcttcctgttaccaagcacaggatgtggggactgggactttaatggctgtgaaacaggtgacgtacgtcagaaacacatcctccgagcaggaggaggtggtggaagcgttgagggaagagatccggatgatgggtcacctcaaccatccaaacatcatccggatgctgggggccacgtgcgagaagagcaactacaacctcttcattgagtggatggcgggaggatctgtggctcacctcttgagtaaatacggagctttcaaggagtcagtcgtcattaactacactgagcagttactgcgtggcctttcctatctccacgagaaccagatcattcacagagacgtcaaaggtgccaacctgctcattgacagcaccggtcagaggctgagaattgcagactttggagctgctgccaggttggcatcaaaaggaaccggtgcaggagagttccagggacagttactggggacaattgcattcatggcgcctgaggtcctaagaggtcagcagtatggtaggagctgtgatgtatggagtgttggctgcgccattatagaaatggcttgtgcaaaaccaccttggaatgcagaaaaacactccaatcatctcgccttgatatttaagattgctagcgcaactactgcaccgtccatcccgtcacacctgtccccgggtctgcgcgacgtggccgtgcgctgcttagaacttcagcctcaggaccggcctccgtccagagagctgctgaaacatccggtcttccgtaccacgtggtagttaattgttcagatcagctctaatggagacaggatatcgaaccgggagagagaaaagagaacttgtgggcgaccatgccgctaaccgcagccctcacgccactgaacagccagaaacggggccagcggggaaccgtacctaagcatgtgattgacaaatcatgacctgtacctaagctcgatatgcagacatctacagctcgtgcaggaactgcacaccgtgcctttcacaggactggctctgggggaccaggaaggcgatggagtttgcatgactaaagaacagaagcataaatttatttttggagcactttttcagctaatcagtattaccatgtacatcaacatgcccgccacatttcaaactcagactgtcccagatgtcaagatccactgtgtttgagtttgtttgcagttccctcagcttgctggtaattgtggtgttttgttttcgatgcaaatgtgatgtaatattcttattttctttggatcaaagctggactgaaaattgtactgtgtaattatttttgtgtttttaatgttatttggtactcgaattgtaaataacgtctactgctgtttattccagtttctactacctcaggtgtcctatagatttttcttctaccaaagttcactctcagaatgaaattctacgtgctgtgtgactatgactcctaagacttccagggcttaagggctaactcctattagcaccttactatgtaagcaaatgctacaaaaaaaaaaaaaaaaa

SEQ ID NO: 2 - protein, 672 aa, Homo sapiens
MVTAVPAVFSKLVTMLNASGSTHFTRMRRRLMAIADEVEIAEVIQLGVEDTVDGHQDSLQAVAPTSCLENSSLEHTVHREKTGKGLSATRLSASSEDISDRLAGVSVGLPSSTTTEQPKPAVQTKGRPHSQCLNSSPLSHAQLMFPAPSAPCSSAPSVPDISKHRPQAFVPCKIPSASPQTQRKFSLQFQRNCSEHRDSDQLSPVFTQSRPPPSSNIHRPKPSRPVPGSTSKLGDATKSSMTLDLGSASRCDDSFGGGGNSGNAVIPSDETVFTPVEDKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDAEWLKGQQIGLGAFSSCYQAQDVGTGTLMAVKQVTYVRNTSSEQEEVVEALREEIRMMGHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVAVRCLELQPQDRPPSRELLKHPVFRTTW

SEQ ID NO: 3 - protein, 1493 aa, Homo sapiens
MAAAAGDRASSSGFPGAAAASPEAGGGGGGGGALQGSGAPAAGAAGLLREPGSAGRERADWRRRQLRKVRSVELDQLPEQPLFLAAASPPCPSTSPSPEPADAAAGASRFQPAAGPPPPGAASRCGSHSAELAAARDSGARSPAGAEPPSAAAPSGREMENKETLKGLHKMEDRPEERMIREKLKATCMPAWKHEWLERRNRRGPVVVKPIPIKGDGSEVNNLAAEPQGEGQAGSAAPAPKGRRSPSPGSSPSGRSVKPESPGVRRKRVSPVPFQSGRITPPRRAPSPDGFSPYSPEETSRRVNKVMRARLYLLQQIGPNSFLIGGDSPDNKYRVFIGPQNCSCGRGAFCIHLLFVMLRVFQLEPSDPMLWRKTLKNFEVESLFQKYHSRRSSRIKAPSRNTIQKFVSRMSNSHTLSSSSTSTSSSENSIKDEEEQMCPICLLGMLDEESLTVCEDGCRNKLHHHCMSIWAEECRRNREPLICPLCRSKWRSHDFYSHELSSPVESPASLRAVQQPSSPQQPVAGSQRRNQESSFNLTHFGTQQIPSAYKDLAEPWIQVFGMELVGCLFSRNWNVREMALRRLSHDVSGALLLANGESTGNSGGGSGGSLSAGAASGSSQPSISGDVVEACCSVLSIVCADPVYKVYVAALKTLRAMLVYTPCHSLAERIKLQRLLRPVVDTILVKCADANSRTSQLSISTVLELCKGQAGELAVGREILKAGSIGVGGVDYVLSCILGNQAESNNWQELLGRLCLIDRLLLEFPAEFYPHIVSTDVSQAEPVEIRYKKLLSLLTFALQSIDNSHSMVGKLSRRIYLSSARMVTAVPAVFSKLVTMLNASGSTHFTRMRRRLMAIADEVEIAEVIQLGVEDTVDGHQDSLQAVAPTSCLENSSLEHTVHREKTGKGLSATRLSASSEDISDRLAGVSVGLPSSTTTEQPKPAVQTKGRPHSQCLNSSPLSHAQLMFPAPSAPCSSAPSVPDISKHRPQAFVPCKIPSASPQTQRKFSLQFQRNCSEHRDSDQLSPVFTQSRPPPSSNIHRPKPSRPVPGSTSKLGDATKSSMTLDLGSASRCDDSFGGGGNSGNAVIPSDETVFTPVEDKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDAEWLKGQQIGLGAFSSCYQAQDVGTGTLMAVKQVTYVRNTSSEQEEVVEALREEIRMMGHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVAVRCLELQPQDRPPSRELLKHPVFRTTW

SEQ ID NO: 4 - protein, 1493 aa, Rattus norvegicus
MAAAAGDRASSSGFPGAAAASPEAGGGGGALQGSGAPAAGAGLLRETGSAGRERADWRRQQLRKVRSVELDQLPEQPLFLTASPPCPSTSPSPEPADAAAGASGFQPAAGPPPPGAASRCGSHSAELAAARDSGARSPAGAEPPSAAAPSGREMENKETLKGLHKMDDRPEERMIREKLKATCMPAWKHEWLERRNRRGPVVVKPIPIKGDGSEMSNLAAELQGEGQAGSAAPAPKGRRSPSPGSSPSGRSGKPESPGVRRKRVSPVPFQSGRITPPRRAPSPDGFSPYSPEETSRRVNKVMRARLYLLQQIGPNSFLIGGDSPDNKYRVFIGPQNCSCGRGTFCIHLLFVMLRVFQLEPSDPMLWRKTLKNFEVESLFQKYHSRRSSRIKAPSRNTIQKFVSRMSNCHTLSSSSTSTSSSENSIKDEEEQMCPICLLGMLDEESLTVCEDGCRNKLHHHCMSIWAEECRRNREPLICPLCRSKWRSHDFYSHELSSPVDSPTSLRGVQQPSSPQQPVAGSQRRNQESNFNLTHYGTQQIPPAYKDLAEPWIQAFGMELVGCLFSRNWNVREMALRRLSHDVSGALLLANGESTGTSGGGSGGSLSAGAASGSSQPSISGDVVEAFCSVLSIVCADPVYKVYVAALKTLRAMLVYTPCHSLAERIKLQRLLRPVVDTILVKCADANSRTSQLSISTLLELCKGQAGELAVGREILKAGSIGVGGVDYVLSCILGNQAESNNWQELLGRLCLIDRLLLEISAEFYPHIVSTDVSQAEPVEIRYKKLLSLLAFALQSIDNSHSMVGKLSRRIYLSSARMVTTVPPLFSKLVTMLSASGSSHFARMRRRLMAIADEVEIAEVIQLGSEDTLDGQQDSSQALAPPRYPESSSLEHTAHVEKTGKGLKATRLSASSEDISDRLAGVSVGLPSSATTEQPKPTVQTKGRPHSQCLNSSPLSPPQLMFPAISAPCSSAPSVPAGSVTDASKHRPRAFVPCKIPSASPQTQRKFSLQFQRTCSENRDSEKLSPVFTQSRPPPSSNIHRAKASRPVPGSTSKLGDASKNSMTLDLNSASQCDDSFGSGSNSGSAVIPSEETAFTPAEDKCRLDVNPELNSSIEDLLEASMPSSDTTVTFKSEVAVLSPEKAESDDTYKDDVNHNQKCKEKMEAEEEEALAIAMAMSASQDALPIVPQLQVENGEDIIIIQQDTPETLPGHTKANEPYREDTEWLKGQQIGLGAFSSCYQAQDVGTGTLMAVKQVTYVRNTSSEQEEVVEALREEIRMMSHLNHPNIIRMLGATCEKSNYNLFIEWMAGASVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVALRCLELQPQDRPPSRELLKHPVFRTTW

SEQ ID NO: 5 - protein, 1034 aa, Homo sapiens
  Xaa′s at postions 19,20,30,52-55,57,58,60,61,68,159,164,174-305,336, 339,341,344,351,352,355,375,519,555,556,691,692,69 5-739,767,769,777-814,819,827,834,842,864,870,875, and 890 may be any amino acid
NKLHHHCMSIWAEECRRNXXPLICPLCRSXWRSHDFYSHELSSPVDSPSSLXXXXQXXVXXHPLAGSXRRNQESNFNLTHYGTQQIPPAYKDLAEPWIQVFGMELVGCLFSRNWNVREMALRRLSHDVSGALLLANGESTGNSGGSSGSSPSGGATSGXSQTSXSGDVVEACCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXPAEFYPHIVSTDVSQAEPVEIRYKKLLSLLXFAXKXIDXSHSMVGXXSRXDISLLCYDDGRSAVCFPSWXPCLMLLGSTHFTRMRRRLMAIADEVEIAEVIQLGEVDTVDGHQDSLRALAPASCRENSSLEHTVHREKTGKGLSATRLSTSSEEISDRLAGVSVGFPSSTTTEQPKPAVQTKGRPHSQCLNSSPLSHAQLMFPAPSAPCSSAPXVPDISKHRPQAFVPCKILPHLPQTQRKFSLQFQRNXXEHRDQTQLSPVFTQSQDPTSSNIHRPKPDRPAPGSTSKLGDATKSSMTLDLGQCSRCDDSFGGGGNSGNAVIPSDETVFTPVEDKCRLDVNTELNSSIEDLLEASMPSSDTTVTFKSEVAVLSPEKAENDDTYKXXVYXXXXXXXXXXXXXXXXXXXXXXXXXXXXXXXXXXXXXXXXXXXXXVIQQDTPETLPGHTKAKQPYREDAEWLXGXQIGLGHFXXXXXXXXXXXXXXXXXXXXXXXXXXXXXXXXXXXXXXEEIRXMSHLNHPXIIRMLGXTGKKSNYXLFIEWMAGGSVAHLLSKYGAFXESVVIXYTEQXLRGLSYLHENQIIHXDVKGANLLIDXTGXRLRIADFGAAAXLASKGXGAGEFQGQLXGTIAFMAPEVXRGXQYGRSCDVWSVGCAIIEMACAKPPWNAEKHSNHLALIKKIASATTAPSIPSHLSPGLRNVALRCLELQPQDRPPSRELLKHPVFRTTX

SEQ ID NO: 6 - protein, 4 aa, synthetic construct
DEVE

SEQ ID NO: 7 - protein, 4 aa, synthetic construct
DTVD

SEQ ID NO: 8 - protein, 15 aa, synthetic construct
DRPPSRELLKHPVER

SEQ ID NO: 9 - protein, 5 aa, synthetic construct
PPPSS

SEQ ID NO: 10 - protein, 4 aa, synthetic construct
YVAD

SEQ ID NO: 11 - protein, 4 aa, synthetic construct
DEVD

SEQ ID NO: 12 - protein, 11 aa, synthetic construct
MGYPYDVDYAS

SEQ ID NO: 13 - DNA, 5253 bp, Mus musculus
  CDS (15)..(4493)
gcccgcgagagaaaatggcggcggcggcgggcgatcgcgcctcgtcgtcgggattcccgggcgccgcggcggcgagtcccgaggcgggcggcggcggcggaggaggaggagctctccagggaagcggcgcgcccgcagcgggcgcggcggggctgctgcgggagcctggcagcgcgggccgcgagcgcgcggactggcggcggcggcacgtgcgcaaagtgcggagtgtggagctggaccagctgccggagcagccgctcttcctcgccgccgcctcgccgccctgcccatctacttccccgtcgccggagcccgcggacgcggctgcaggagcgagtcgcttccagcccgcggcgggaccgccacccccgggagcggcgagtcgctgcggctcccactctgccgagctggcggccgcgcgggacagcggcgcccggagccccgcgggggcggagccgccctctgcagcggccccctccggtcgagagatggagaataaagaaaccctcaaaggactgcacaagatggaggatcgcccggaggagagaatgatccgggagaagctcaaggcgacctgtatgccggcctggaagcacgagtggttggagaggaggaacaggagaggccctgtggtggtgaagccaatccctattaaaggagatggatctgaagtgaataacttggcagctgagccccagggagagggccaggcaggttccgctgcaccagcccccaagggccgacgaagcccatctcctggcagctctccgtcagggcgctcggtgaagccggaatccccaggagtaagacggaaacgagtgtccccggtgcctttccagagtggcagaatcacaccaccccgaagagccccatcaccggatggcttctccccgtacagcccagaggagacgagccgccgcgtgaacaaagtgatgagagccaggctgtacctgctgcagcagataggacccaactctttcctgattggaggagacagtccagacaataaataccgggtgtttattgggccacagaactgcagctgtgggcgtggagcattctgtattcacctcttgtttgtcatgctccgggtgtttcagctagaaccctctgaccccatgttatggagaaaaactttaaaaaatttcgaggttgagagtttgttccagaaataccacagtaggcgtagctcgagaatcaaagctccatcccggaacaccatccagaagtttgtgtcacgcatgtcaaattctcacacactgtcatcgtctagcacatccacatctagttcagaaaacagcatcaaggatgaagaggagcagatgtgtcccatctgcttgctgggcatgctggatgaggagagcctgactgtgtgtgaagatggctgcaggaacaagctgcaccaccattgcatgtccatctgggcggaagagtgtagaagaaatagagagcctttaatatgtcccctttgtagatctaagtggagatcccatgacttctacagccatgagttatcaagccccgtggagtcccccgcctccctgcgagctgtccagcagccatcctccccgcagcagcccgtggccggatcacagcggaggaatcaggagagcagttttaaccttactcattttggaacccagcagattccttccgcttacaaagatttggccgagccatggattcaggtgtttggaatggaactcgttggctgcttattctctagaaactggaacgtaagggaaatggcccttaggcgtctttcccacgacgttagtggggccctgttgttggcaaacggggagagcactggaaactctggaggcggcagtgggggcagcttaagcgcgggagcggccagcgggtcctcccagcccagcatctcaggggatgtggtggaggcgtgctgcagtgtcctgtctatagtctgcgctgaccctgtctacaaagtgtacgttgctgctttaaaaacattgagagccatgctggtatacactccttgccacagtctggcagaaagaatcaaacttcagagactcctccggccagttgtagacactatccttgtcaagtgtgcagatgccaacagccgcacgagtcagctgtccatatctacagtgctggaactctgcaagggccaagcaggagagctggcggttgggagagaaatacttaaagctgggtccatcggggttggtggtgtcgattacgtcttaagttgtatccttggaaaccaagctgaatcaaacaactggcaagaactgctgggtcgcctctgtcttatagacaggttgctgttggaatttcctgctgaattctatcctcatattgtcagtactgatgtctcacaagctgagcctgttgaaatcaggtacaagaagctgctctccctcttaacctttgccttgcaatccattgacaattcccactcgatggttggcaagctctctcggaggatatatctgagctctgccaggatggtgaccgcagtgcccgctgtgttttccaagctggtaaccatgcttaatgcttctggctccacccacttcaccaggatgcgccggcgtctgatggctatcgcggatgaggtagaaattgccgaggtcatccagctgggtgtggaggacactgtggatgggcatcaggacagcttacaggccgtggcccccaccagctgtctagaaaacagctcccttgagcacacagtccatagagagaaaactggaaaaggactaagtgctacgagactgagtgccagctcggaggacatttctgacagactggccggcgtctctgtaggacttcccagctcaacaacaacagaacaaccaaagccagcggttcaaacaaaaggcagaccccacagtcagtgtttgaactcctcccctttgtctcatgctcaattaatgttcccagcaccatcagccccttgttcctctgccccgtctgtcccagatatttctaagcacagaccccaggcatttgttccctgcaaaataccttccgcatctcctcagacacagcgcaagttctctctacaattccagaggaactgctctgaacaccgagactcagaccagctctccccagtcttcactcagtcaagacccccaccctccagtaacatacacaggccaaagccatcccgacccgttccgggcagtacaagcaaactaggggacgccacaaaaagtagcatgacacttgatctgggcagtgcttccaggtgtgacgacagctttggcggcggcggcaacagtggcaacgccgtcatacccagcgacgagacagtgttcacgccggtggaggacaagtgcaggttagatgtgaacaccgagctcaactccagcatcgaggaccttcttgaagcatccatgccttcaagtgacacgacagtcactttcaagtccgaagtcgccgtcctctctccggaaaaggccgaaaatgacgacacctacaaagacgacgtcaatcataatcaaaagtgcaaagaaaagatggaagctgaagaggaggaggctttagcgatcgccatggcgatgtcagcgtctcaggatgccctccccatcgtccctcagctgcaggtggaaaatggagaagatattatcatcattcagcaggacacaccagaaactcttccaggacataccaaagcgaaacagccttacagagaagacgctgagtggctgaaaggccagcagataggcctcggagcattttcttcctgttaccaagcacaggatgtggggactgggactttaatggctgtgaaacaggtgacgtacgtcagaaacacatcctccgagcaggaggaggtggtggaagcgttgagggaagagatccggatgatgggtcacctcaaccatccaaacatcatccggatgctgggggccacgtgcgagaagagcaactacaacctcttcattgagtggatggcgggaggatctgtggctcacctcttgagtaaatacggagctttcaaggagtcagtcgtcattaactacactgagcagttactgcgtggcctttcctatctccacgagaaccagatcattcacagagacgtcaaaggtgccaacctgctcattgacagcaccggtcagaggctgagaattgcagactttggagctgctgccaggttggcatcaaaaggaaccggtgcaggagagttccagggacagttactggggacaattgcattcatggcgcctgaggtcctaagaggtcagcagtatggtaggagctgtgatgtatggagtgttggctgcgccattatagaaatggcttgtgcaaaaccaccttggaatgcagaaaaacactccaatcatctcgccttgatatttaagattgctagcgcaactactgcaccgtccatcccgtcacacctgtccccgggtctgcgcgacgtggccgtgcgctgcttagaacttcagcctcaggaccggcctccgtccagagagctgctgaaacatccggtcttccgtaccacgtggtagttaattgttcagatcagctctaatggagacaggatatgcaaccgggagagagaaaagagaacttgtgggcgaccatgccgctaaccgcagccctcacgccactgaacagccagaaacggggccagcggggaaccgtacctaagcatgtgattgacaaatcatgacctgtacctaagctcgatatgcagacatctacagctcgtgcaggaactgcacaccgtgcctttcacaggactggctctgggggaccaggaaggcgatggagtttgcatgactaaagaacagaagcataaatttatttttggagcactttttcagctaatcagtattaccatgtacatcaacatgcccgccacatttcaaactcagactgtcccagatgtcaagatccactgtgtttgagtttgtttgcagttccctcagcttgctggtaattgtggtgttttgttttcgatgcaaatgtgatgtaatattcttattttctttggatcaaagctggactgaaaattgtactgtgtaattatttttgtgtttttaatgttatttggtactcgaattgtaaataacgtctactgctgtttattccagtttctactacctcaggtgtcctatagatttttcttctaccaaagttcactctcagaatgaaattctacgtgctgtgtgactatgactcctaagacttccagggcttaagggctaactcctattagcaccttactatgtaagcaaatgctacaaaaaaaaaaaaaaaaaa